(19) United States
(12) Patent Application Publication
(10) Pub. No.: US 2025/0281500 A1
(43) Pub. Date: Sep. 11, 2025

(54) FUSED AMINO PYRIMIDINE COMPOUNDS AND PRIDOPIDINE FOR TREATMENT OF HUNTINGTON’S DISEASE

(71) Applicant: Ovid Therapeutics Inc., New York, NY (US)

(72) Inventors: Patrick Sarmiere, New York, NY (US); Toshiya Nishi, New York, NY (US); Jayanta Mukherjee, New York, NY (US)

(21) Appl. No.: 19/071,880

(22) Filed: Mar. 6, 2025

Related U.S. Application Data
(60) Provisional application No. 63/561,937, filed on Mar. 6, 2024.

(51) Int. Cl. A61K 31/5377; A61K 31/519; A61P 25/28

(52) U.S. Cl. CPC A61K 31/5377; A61K 31/519; A61P 25/28

(57) ABSTRACT

Compositions and methods for treating Huntington's disease with a compound of Formula (I), or a pharmaceutically acceptable salt thereof, in combination with pridopidine, or a pharmaceutical salt thereof, are provided. The compositions and methods may be used to improve one or more symptoms of Huntington's disease.
Formula (I):

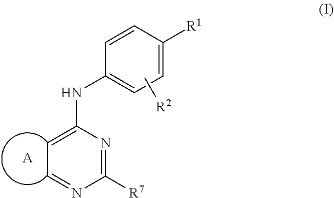

or pharmaceutically acceptable salts thereof, wherein R¹, R², R⁷ and ring A have any of the meanings herein defined in the description.

### CROSS REFERENCE TO RELATED APPLICATION

This application claims benefit of and priority to the U.S. Provisional Application No. 63/561,937, filed on Mar. 6, 2024, which is incorporated herein by reference in its entirety.

### TECHNICAL FIELD

Treatment of Huntington's disease with fused amino pyrimidine compounds and pridopidine.

### BACKGROUND

According to the National Institutes of Health, National Institute of Neurological Disorders and Stroke (https://www.ninds.nih.gov/health-information/disorders/huntingtons-disease), Huntington's disease (HD) is an inherited disorder that causes neurons in parts of the brain to gradually break down and die. The disease attacks areas of the brain that help to control voluntary (intentional) movement, as well as other areas. People living with HD develop uncontrollable dance-like movements (chorea) and abnormal body postures, as well as problems with behavior, emotion, thinking, and personality. HD is a neurodegenerative autosomal dominant disorder, characterized by involuntary choreatic movements with cognitive and behavioral disturbances. See, Ajitkumar and De Jesus, Huntington Disease, [Updated 2023 Aug. 23], In: StatPearls [Internet], Treasure Island (FL: StatPearls Publishing; 2024 January, available from: https://www.ncbi.nlm.nih.gov/books/NBK559166. It occurs as a result of cytosine, adenine, and guanine (CAG) trinucleotide repeats on the short arm of chromosome 4p16.3 in the Huntingtin (HTT) gene. Id. This mutation leads to an abnormally long expansion of the polyglutamine in the HTT protein, which leads to neurodegeneration. Id. The expansion also causes the HTT protein to be more prone to aggregation and accumulation that mitigates protein folding. Id. The primary feature is the degeneration of neurons in the putamen, caudate as well as the cerebral cortex. Id. The preferential degeneration of the enkephalin-containing medium spiny neurons in the basal ganglia in the indirect pathway provides the basis for chorea. Id. Additional loss of substance-P containing medium spiny neurons in the direct pathway results in the development of dystonia and akinesia. Id. Clinical studies have demonstrated that HD is characterized by pre-synaptic as well as post-synaptic dopamine-related dysfunctions with reduction in striatal dopamine synthesis, dopamine storage, dopamine transporter binding, and both dopamine D1 and D2 receptor binding. Waters et al., J Huntingtons Dis. 2018; 7(1):1-16. doi: 10.3233/JHD-170267. The hallmark neuropathological feature of HD is degeneration of medium spiny neurons (MSNs) in the striatum. Id. Motor control is exerted by dopamine released from the nigrostriatal pathway, modulating the activity of MSNs involved in the facilitation of movement, and inhibition of unwanted movement. Id. MSNs are GABAergic neurons, expressing high densities of dopamine receptors, and a progressive decline in striatal dopamine receptor density is one of the earliest findings in patients with HD. Id.

According to Ajitkumar and De Jesus, supra, the signs and symptoms of HD classically consist of motor, cognitive, and psychiatric disturbances. Other less common features include weight loss, sleep disturbances, and autonomic nervous system dysfunction. Motor disturbances, which are progressive, include characteristic unwanted involuntary movements which initially begin in the distal extremities and then spread gradually to the more proximal and axial muscles and are of greater amplitude. Early in the disease, they are mostly hyperkinetic with involuntary chorea. In later stages hypokinesia with bradykinesia and dystonia predominate. Dysarthria and dysphagia develop during the course of the disease, which could lead to aspiration in patients, with pneumonia being a common cause of death. Dystonia, characterized by increased muscle tone with slower movements, leads to abnormal posturing. Other involuntary features include tics and cerebellar signs such as ataxia. Pyramidal signs such as the Babinski sign are present. The motor disturbance on daily activities progresses over time and can lead to difficulties in walking, standing, and frequent falls. Behavioral and psychiatric symptoms are present very early in the disease. Initially, patients may present with poor attention, impulsivity, and irritability which can lead to outbursts of anger and aggression. Subsequently, there is an emotional blandness with prominent apathy, loss of intuition, and creativity. Depression is also commonly reported. Psychosis can appear in later stages. Cognitive decline is one of the main features of HD and could also be present before the onset of motor disturbances. The cognitive changes are more prominent for executive functions with patients finding difficulty in organizing, multitasking, and planning. These symptoms then progress with more cognitive deficits leading to dementia. Other secondary symptoms include gait abnormalities, eye movement abnormalities, and seizures which are only present in the juvenile variant, occurring in 30 to 50% of patients before the age of 10.

There is no cure for HD. Id. However, many therapeutic options exist for treating signs and symptoms with the aim of improving the quality of life. Treatment is mainly pharmacological as well as supportive. Id. It is well established that pharmacological treatments that modify dopaminergic function impact on the motor symptoms of HD. Waters et al., supra. Numerous medications from different classes are prescribed off-label to ameliorate the motor symptoms associated with ID. Id. These medications include, e.g., antidopaminergic drugs, energy metabolites, benzodiazepines, and glutamate-modifying drugs (riluzole and amantadine). Id. The most frequently prescribed drugs for chorea in HD include tetrabenazine, olanzapine, risperidone, tiapride, quetiapine, and aripiprazole. Chen et al. Front Neurol. 2021 May 13; 12:658123. doi: 10.3389/fneur.2021.658123. Tetrabenazine (TBZ), which is an inhibitor of presynaptic vesicular monoamine transporter type 2 (VMAT2) along with a synaptic vascular amine transport inhibitor and provides sustained reductions in dopaminergic neurotransmission, is the only drug specifically licensed by the US FDA to treat chorea. Id. However, the evidence for the effectiveness of such treatments is poor. Waters et al., supra.

Pridopidine (4-[3-(methylsulfonyl)phenyl]-1-propyl-piperidine) belongs to the new class of D2 receptor antagonists and is also called a dopaminergic stabilizer. Chen et al., supra. Pridopidine increases striatal dopaminergic transmission when the dopaminergic tone is low and inhibits the stimulatory actions of dopamine when dopaminergic activity is high. Id. Pridopidine can reverse and improve behavioral states in a concentration-dependent manner without having major effects on normal mental activity. Id. Two large clinical trials in patients with manifest HD indicated that pridopidine can improve motor function. Waters et al., supra.

There are no approved or established treatments for general improvement of the multifaceted motor symptoms of HD. See, Waters et al., supra. Hence, there is a significant unmet medical need to ameliorate both positive and negative motor symptoms of HD and to slow or halt the progression. Id.

Fused amino pyridine compounds and pharmaceutically acceptable salts thereof are disclosed in WO2021/180952, incorporated herein by reference. As described in WO2021/180952, these compounds and their pharmaceutically acceptable salts selectively modulate KCC2 and are used to treat or prevent KCC2 mediated disease, including neurological disorders. As described in the OMIM database, the LC12A5 gene encodes the neuronal KCC2 channel that is the major extruder of intracellular chloride in mature neurons. In the presence of low intraneuronal chloride, the binding of GABA and glycine to their ionotropic receptors results in chloride influx with subsequent hyperpolarization contributing to neuronal inhibition.

Hyperpolarizing GABA_{A}R currents are critically dependent upon efficient Cl⁻ extrusion, which is facilitated by KCC2. Conditional inactivation of KCC2 in the hippocampus leads to neuronal Cl⁻ accumulation and depolarizing GABA_{A}R currents. These deficits in inhibition parallel the development of spontaneous seizures, neuronal apoptosis and reactive astrocytosis (Kelley et al., EBioMedicine 32, 62-71 (2018)). GABA_{A} signaling is a major inhibitory neurotransmitter mechanism in the adult brain and consequently KCC2 has a key role in normal neurodevelopment and various neurological disorders. Decreased activity of KCC2 has been implicated in the pathogenesis of neurological disorders including epilepsy (Galanopoulou et al, Epilepsia 2007; 48:14-18; Huberfield et al, The Journal of Neuroscience (2007) 27, 9866-9873), neuropathic pain (Price et al, Curr Top Med Chem 2005; 5:547-555), Rett's syndrome (Tang et al, 2019, Translational Medicine, 11(503)), autism (Tyzio et al, Science 343, 675-679, Merner et al, Frontiers in cellular neuroscience 9, 2015), mental disorders, spinal cord injury (Boulenguez et al, Nature Medicine 2010, 16, 302-307) and conditions in which there is neuronal hyperexcitability such as ALS (Fuchs et al, Journal of Neuropathology & Experimental Neurology, Volume 69, Issue 10, October 2010, Pages 1057-1070).

Direct modulation of KCC2 by interaction with small molecules has been reported. Delpire et al (Proc Natl Acad Sci USA. 2009 Mar. 31; 106(13): 5383-5388) describe an assay to identify small molecule inhibitors of KCC2 and Zhang et al (Journal of Biomolecular Screening 15(2): 2010) describe an assay used to identify positive modulators of KCC2.

### SUMMARY

Methods and compositions for treating Huntington's disease are provided and, in embodiments, include administering to a subject diagnosed with Huntington's disease an effective amount of a compound according to Formula (I):

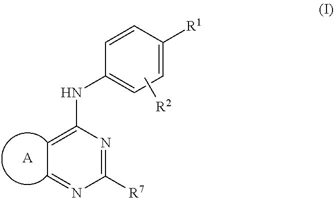

or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;

R² is selected from —H; -halo; and —C₁₋₃alkyl optionally substituted with 1, 2 or 3 substituents selected from —F and —CF₃;
A is selected from

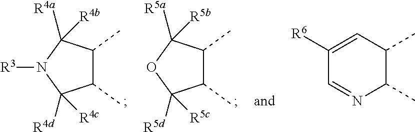

or a N-oxide thereof;
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
in combination with an effective amount of pridopidine, or a pharmaceutically acceptable salt thereof.

In embodiments, methods of treating a subject diagnosed with Huntington's disease described herein include administering a compound according to Formula (I), or a pharmaceutically acceptable salt thereof, in combination with pridopidine, or a pharmaceutically acceptable salt thereof, to the subject diagnosed with Huntington's disease to provide improvement in one or more symptoms of Huntington's disease. In embodiments, methods of treating Huntington's disease include administering a compound according to Formula (I), or a pharmaceutically acceptable salt thereof, in combination with pridopidine, or a pharmaceutically acceptable salt thereof, to a subject diagnosed with Huntington's disease to provide improvement in next day functioning of the subject. In embodiments, provided herein is a compound of Formula (I), or a pharmaceutically acceptable salt thereof, in combination with pridopidine, or a pharmaceutically acceptable salt thereof, for use in treating Huntington's disease in a subject. In embodiments, provided herein is a compound of Formula (I), or a pharmaceutically acceptable salt thereof, in combination with pridopidine, or a pharmaceutically acceptable salt thereof, for use in providing improvement in next day functioning of a subject with Huntington's disease. In embodiments, provided herein is a compound of Formula (I), or a pharmaceutically acceptable salt thereof, in combination with pridopidine, or a pharmaceutically acceptable salt thereof, for the manufacture of a medicament for treatment of Huntington's disease. In embodiments, Huntington's disease is being treated.

In embodiments, a compound of Formula (I) is Compound A:

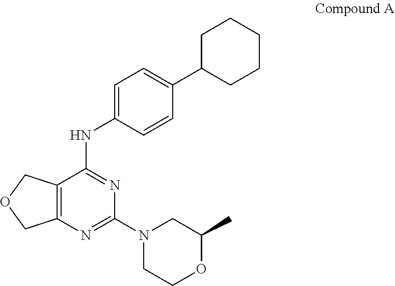 [SMILES: C[C@@H]1CN(c2nc3c(c(Nc4ccc(C5CCCCC5)cc4)n2)COC3)CCO1]

In embodiments, a compound of Formula (I) is Compound B:

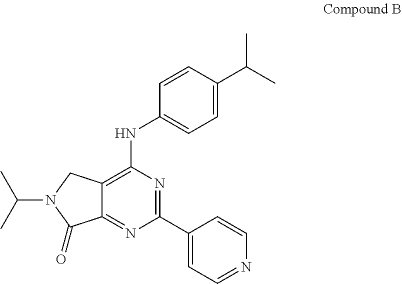 [SMILES: CC(C)c1ccc(Nc2nc(-c3ccncc3)nc3c2CN(C(C)C)C3=O)cc1]

In embodiments, a compound of Formula (I) is Compound C:

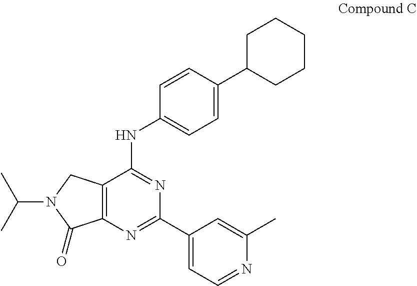 [SMILES: Cc1cc(-c2nc(Nc3ccc(C4CCCCC4)cc3)c3c(n2)C(=O)N(C(C)C)C3)ccn1]

In embodiments, a compound of Formula (I) is Compound D:

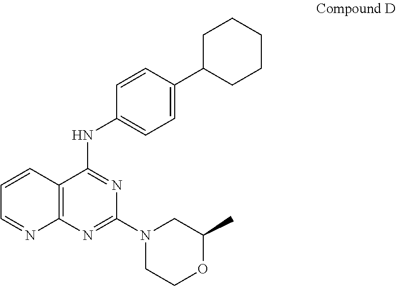 [SMILES: C[C@@H]1CN(c2nc(Nc3ccc(C4CCCCC4)cc3)c3cccnc3n2)CCO1]

In embodiments, a compound of Formula (I) is Compound E:

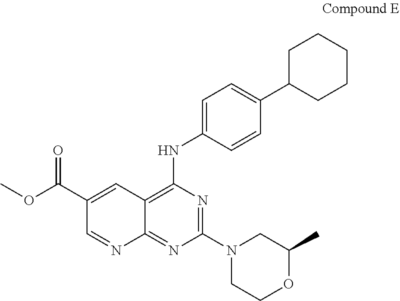 [SMILES: COC(=O)c1cnc2nc(N3CCO[C@H](C)C3)nc(Nc3ccc(C4CCCCC4)cc3)c2c1]

In embodiments, a compound of Formula (I) is Compound F:

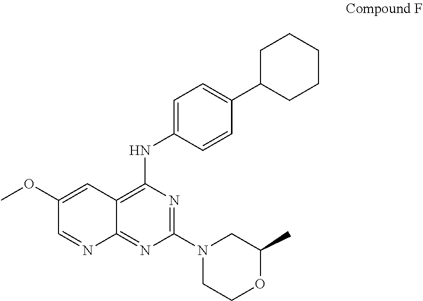 [SMILES: COc1cnc2nc(N3CCO[C@H](C)C3)nc(Nc3ccc(C4CCCCC4)cc3)c2c1]

In embodiments, a compound of Formula (I) is Compound G:

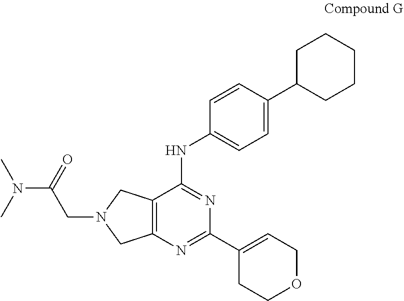 [SMILES: CN(C)C(=O)CN1Cc2nc(C3=CCOCC3)nc(Nc3ccc(C4CCCCC4)cc3)c2C1]

### BRIEF DESCRIPTION OF THE FIGURES

FIG. 1A is a drug screening workflow chart illustrating the steps taken to identify KCC2 direct activators, a multi-tiered program starting from a compound library of 1.3 million compounds. Identification of a subseries of fused pyrimidine ring compounds, denoted as subseries A (SSA) were identified as lead-hits from the compound library screen.

FIG. 1B depicts immunoblotting results for HEK-293 cells expressing KCC2 exposed to vehicle (−) or 30 μM SSA1 (+) for 90 min and then heated to 37-58° C. Soluble fractions were subsequently immunoblotted with KCC2 and GAPDH antibodies.

FIG. 1C is a graph depicting thermal aggregation curves for KCC2 under control conditions and in the presence of SSA-0126. *=significantly different to control p<0.001; t-test; n=4 transfections. The level of remaining soluble native KCC2 at each temperature was normalized to that seen at 37° C. (IxI37° C.).

FIG. 1D is a graph depicting isothermal dose response curves constructed for destabilization of KCC2 by SSA1 at 50° C., n=4 transfections

FIG. 1E shows the structure of Compound A (also referred to as CmpA).

FIG. 1F is a graph showing the effects of Compound A on KCC2 activity expressed in HEK-293 cells as measured using TI flux. Data is normalized to values seen with vehicle (100%), n=3.

FIG. 1G is a chart showing the EC₅₀ of Compound A for KCC2, KCC2-S940A, KCC3, KCC4 and NKCC1 as measured in expressing HEK-293 cells, n=4 transfections. In all panels p values were determined using unpaired t-tests with Welch's correction.

FIG. 2A depicts three graphs showing E_{Gly} values versus time as measured from HEK-293 cells expressing KCC2 together with GlyRα1 using perforated patch clamp recordings. Individual shifts in E_{Gly} are shown for cells incubated with Compound A (0.3 and 3 μM) or vehicle (V) for 15 min.

FIG. 2B depicts a first bar graph showing ΔE_{Gly} (mM) by concentration of vehicle and Compound A and a second bar graph showing reduction in [Cl⁻](mM) by concentration of vehicle and Compound A. Mean shifts in E_{Gly} were determined for cells treated with vehicle, p=0.1227, Compound A 0.3 μM (*p=0.0051), and 3 μM (*p=0.0350), respectively, n=5 transfections. Compound A induced changes in [Cl⁻] were calculated from E_{Gly} using the Nernst equation for vehicle (V), (p=0.1227), 300 nM, (*p=0.0051), or 3 μM (*p=0.0350) CmpA, n=5 transfections.

FIG. 2C depicts immunoblotting results and a bar graph showing surface/total KCC2 (% conc.) by concentration of Compound A. HEK-293 cells (UT) or those expressing KCC2 were exposed to V, 0.3 μM, or 3 μM 350 for 15 min and biotinylated with NHS-biotin. Surface and total extracts were subsequently immunoblotted with KCC2 and actin antibodies. The ratio of surface/total KCC2 immunoreactivity was determined and normalized to V (100%) (n=3 transfections).

FIG. 2D depicts immunoblotting results obtained by exposing untransfected HEK-293 cells (U) or those expressing KCC2 to vehicle (V), 0.3, or 3 μM, Compound A, for 15 min and biotinylated with NHS-Biotin. Surface and total extracts were subsequently immunoblotted with KCC2 and actin antibodies. The ratio of surface/total KCC2 immunoreactivity was determined and normalized to V (100%) for 0.3 (*p=0.659) or 3 μM Compound A (*p=0.739), n=3 transfections.

FIG. 2E depicts a first bar graph showing pS940/KCC2 (% concentration) of Compound A (μM) and a second bar graph showing pT1007/KCC2 (% concentration) of Compound A (μM). HEK-293 cells were treated with V (−) or 3 μM Compound A (+) for 15 min. Cell lysates were immunoblotted with KCC2, pS940, KCC2 and actin antibodies. The ratios of pS940/KCC2 (p=0.491) and pT1007/KCC2 (p=0.721) were then compared to V (100%), n=3 transfections. In all panels p values were determined using unpaired t-tests with Welch's correction.

FIG. 3A depicts a graph showing current (pA) versus holding potential (mV) for vehicle and Compound A. 18-21 Div hippocampal neurons were subjected to gramicidin perforated patch clamp recordings in the presence of bumetanide (10 μM) and TTX (500 nM). After attaining perforation, cultures were exposed to 300 nM Compound A, or V (1% BCD) for 15 min. E_{GABA} was then determine using voltage ramps protocols and representative current-voltage (I-V) plots are shown for neurons at 0 (dark gray) and 15 (light gray) min treatment for V and Compound A treated neurons.

FIG. 3B depicts a first bar graph showing neuronal E_{GABA} values by exposure to vehicle (V) over time and a second bar graph defining neuronal E_{GABA} values by exposure to Compound A over time. E_{GABA} values were measured at 0 and 15 min following treatment with V (p=0.2190) or 300 nM Compound A (*p=0.0104), n=5 cultures.

FIG. 3C depicts a bar graph showing neuronal [Cl⁻] values by exposure to Compound A over time. [Cl⁻] values were calculated from E_{GABA} values for V (p=0.1646) or Compound A (*p=0.0153) treated neurons, n=5 cultures.

FIG. 3D depicts a first bar graph showing neuronal Basal E_{GABA} (mV) upon exposure to vehicle (V) and Compound A, and a second bar graph showing neuronal 11K Δ E_{GABA} upon exposure to vehicle (V) and Compound A. 18-21 Div hippocampal neurons were incubated with 300 nM Compound A or vehicle for 1 h. Neurons were subject to whole cell recording using an intracellular solution containing 30 mM Cl⁻. 5 min later basal E_{GABA} values were determined and compared between treatments, *p=0.0253, n=4 cultures. Neurons were subsequently exposed to 10 μM VU0463271 and the magnitude of the shift in E_{GABA} (VU0463271ΔE_{GABA}) was then determined and compared between groups, *p=0.040 n=4 cultures.

FIG. 3E depicts a first electrograph showing the effect of vehicle on neuronal excitability in C57Bl/6 brain slices and a second electrograph showing the effect of Compound A on neuronal excitability in C57Bl/6 brain slices. C57Bl/6 brain slices were incubated in ACSF supplemented with vehicle or Compound A (1 μM) for 1 h at 34° C., and continuously perfused to ASCF deficient in Mg⁺² (0-Mg) (arrow). Field recordings were then performed within the entorhinal cortex as a means of monitoring neuronal excitability. The green line indicates the onset of the first SLE, while the red indicates the development of LRDs.

FIG. 3F depicts a first bar graph showing the effect of Compound A and Vehicle (V) on latency to first seizure-like events (SLE) (minutes) and a second bar graph showing the effect of Compound A and Vehicle (V) on latency to late recurrent discharges (LRD) (minutes). The time to the 1^{st} SLE, p=0.1582, and to LRDs *p=0.0028, was compared between treatments, n=7 mice. In all panels p values were determined using unpaired t-tests with Welch's correction.

FIG. 4A depicts a bar graph showing accumulation of Compound A in the mouse brain within 30 minutes after intravenous injection of 25 mg/kg Compound A in 5% β-cyclodextrin (BCD). 15 and 60 minutes later drug levels were measured via LC-MS/MS, n=3 mice.

FIG. 4B depicts a bar graph showing accumulation of Compound A in the mouse brain after intravenous injection of 50 mg/kg Compound A in 5% β-cyclodextrin (BCD). 1 and 2 h later drug levels were measured via LC-MS/MS, n=3 mice.

FIG. 4C is a graph showing accumulation of Compound A in the mouse brain within 30 minutes after subcutaneous injection of 50 mg/Kg Compound A in 5% β-cyclodextrin (BCD). Drug accumulation was then measured over a time course of 8 h via LC-MS/MS, n=3-4 mice.

FIG. 4D depicts a first bar graph showing the effect of Compound A and vehicle (V) on distance traveled after mice were dosed with vehicle (V) or 50 mg/kg Compound A and a second bar graph showing the effect of Compound A and vehicle (V) on time spent in a center zone after mice were dosed with vehicle or 50 mg/kg Compound A. 2-3 h after injection, the mice were placed in the center of a 60 cm×60 cm open field and allowed to explore for 10 min. The total distance traveled (p=0.365) and time in the center of the area (p=0.425) were then quantified and compared between treatments using t-tests; n=9 mice.

FIG. 4E depicts a first bar graph showing the effect of Compound A on forelimb clonus induced by pentylenetetrazol (PTZ) after mice were injected intravenously with 25 mg/kg Compound A or vehicle, followed 30 min later with ascending concentrations of PTZ, and a second bar graph showing the effect of Compound A on hindlimb clonus induced by pentylenetetrazol (PTZ) after mice were injected intravenously with 25 mg/kg Compound A or vehicle, followed 30 min later with ascending concentrations of PTZ. Mice were subject to video recording and the doses required to induced fore and hindlimb doses were determined. The respective doses to induced fore (p=0.120) and hindlimb clonus (*p=0.001) were then compared using an unpaired t-tests with Welch's correction n=14 mice.

FIG. 5A depicts an electroencephalogram (EEG) demonstrating anticonvulsant properties of Compound A and its effects on the development of kainic acid (KA) induced SE in mice. Example EEG traces are shown for mice injected with 50 mg/kg Compound A or vehicle (SC) 2 h prior to dosing IP with 20 mg/kg KA (black arrow). 2 h following KA injection mice were dosed with IP with 10 mg/kg diazepam (DZ) (green arrow) and EEG recordings were extended for a further 1 h.

FIG. 5B depicts a first bar graph showing the effect of Vehicle (V) and Compound A on time to first seizure, a second bar graph showing the effect of Vehicle (V) and Compound A on time to SE, and a third bar graph showing the effect of Vehicle (V) and Compound A % total time in epileptiform activity. The time to the 1^{st} seizure (*p=0.0235), the onset of SE (*p=0.0329) and the % total time in epileptiform activity (*p=0.023) were compared between treatment groups, n=9 mice.

FIG. 5C depicts a first graph showing power (μV²) versus frequency (Hz) and reduction of EEG power by Compound A as compared to Vehicle (V) and a second graph (bar graph) showing reduction of total power (μV²) by Compound A as compared to Vehicle (V). EEG recordings were subjected to Fast Fourier transform (FFT) and spectral plots are shown for frequencies between 0-100 Hz for mice treated with vehicle or Compound A 2 h after kainic acid (KA) injection. Total EEG power was then compared between treatments, *p=0.0024, t-test; n=9 mice.

FIG. 5D depicts a first graph showing power (μV²) versus frequency (Hz) and the effect of Vehicle (V) before and after treatment with diazepam (DZ) and a second graph (bar graph) showing the effect of Vehicle (V) on total power (μV²) before and after treatment with diazepam (DZ). EEG recordings from mice pretreated with Vehicle (V) 2 h following kainic acid (KA) injection and 10 min after dosing with diazepam (DZ) were subjected to Fast Fourier transformation (FFT) and spectral plots are shown for frequencies between 0-100 Hz. Total EEG power was then compared *p=0.004, n=9 mice.

FIG. 5E depicts a first graph showing power (μV²) versus frequency (Hz) and the effect of Compound A before and after treatment with diazepam (DZ) and a second graph (bar graph) showing the effect of Compound A on total power (μV²) before and after treatment with diazepam (DZ). EEG recordings from mice pretreated with vehicle 2 h following KA injection and 10 min after dosing with DZ were subject to Fast Fourier transformation (FFT) and spectral plots are shown for frequencies between 0-100 Hz. Total EEG power was then compared p=0.104, n=9 mice. In all panels p values were determined using unpaired t-tests with Welch's correction.

FIG. 6A depicts an electroencephalogram (EEG) for mice injected IP with 20 mg/kg KA (black arrow). 2 h following KA injection mice were dosed IP with 10 mg/kg DZ (gray arrow) and EEG recordings were extended for a further 1 h.

FIG. 6B depicts a first graph showing power (μV²) versus frequency (Hz) and the effect of diazepam (DZ) following KA injection and a second graph showing power (μV²) versus frequency (Hz) and the effect of DZ and Compound A following KA injection. EEG recordings 2 h after KA injection and 30 min following DZ or DZ/Compound A treatment were subject to Fast Fourier transformation (FFT) and spectral plots are shown for frequencies between 0-100 Hz.

FIG. 6C depicts a bar graph showing predrug power (μV²)×10⁻⁶ following KA treatment, diazepam (DZ) treatment and diazepam (DZ)/Compound A treatment. Total EEG power was compared for treatment groups 2 h following KA injection, p=0.3732, n=12 mice.

FIG. 6D depicts a bar graph showing postdrug power (μV²)×10⁻⁶ following KA treatment, diazepam (DZ) treatment and diazepam (DZ)/Compound A treatment. Total EEG power 2 h after KA injection was compared to that 30 min following DZ, or DZ/Compound A treatment, *p=0.0072, n=12 mice.

FIG. 6E depicts a bar graph showing % predrug power following KA treatment, diazepam (DZ) treatment and diazepam (DZ)/Compound A treatment.

FIG. 6F depicts a bar graph showing minutes to suppression following KA treatment, diazepam (DZ) treatment and diazepam (DZ)/Compound A treatment. The latency to seizure suppression was measured following injection of DZ or DZ/CmpA, p=0.0008, n=12 mice. In all panels p values were determined using unpaired t-tests with Welch's correction.

FIG. 6G depicts a bar graph showing % insensitive to diazepam (DZ) treatment alone and to diazepam (DZ)/Compound A treatment together.

FIG. 7A depicts images of mice brain hippocampal sections subjected to TUNEL/DAPI staining after treatment with diazepam (DZ) and diazepam (DZ)/Compound A.

FIG. 7B depicts a bar graph showing number of dead cells/in hippocampal CA1 cells after treatment with Vehicle and Compound A. The number of TUNEL positive neurons in CA1 was compared between treatment groups, *p=0.0029, unpaired t-test with Welch's correction, n=14 sections.

### DETAILED DESCRIPTION

Compositions and methods of treating Huntington's Disease are provided and, in embodiments, include administering to a subject diagnosed with Huntington's Disease an effective amount of a compound according to Formula (I):

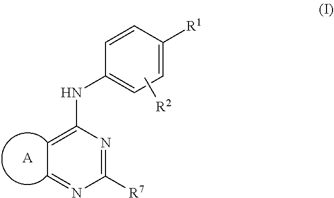

or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from

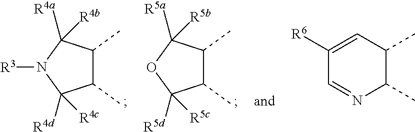

R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)ₙR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
in combination with an effective amount of pridopidine, or a pharmaceutically acceptable salt of pridopidine.

In embodiments, methods of treating Huntington's disease include administering a compound according to Formula (I), or a pharmaceutically acceptable salt thereof, in combination with an effective amount of pridopidine, or a pharmaceutically acceptable salt thereof, to a subject diagnosed with Huntington's disease to provide improvement in one or more symptoms of Huntington's disease. In embodiments, methods of treating Huntington's disease include administering a compound according to Formula (I), or a pharmaceutically acceptable salt thereof, in combination with an effective amount of pridopidine, or a pharmaceutically acceptable salt thereof, to a subject diagnosed with Huntington's disease to provide improvement in next day functioning of the subject. In embodiments, provided herein is a compound of Formula (I), or a pharmaceutically acceptable salt thereof, in combination with an effective amount of pridopidine, or a pharmaceutically acceptable salt thereof, for use in treating Huntington's disease in a subject. In embodiments, provided herein is a compound of Formula (I), or a pharmaceutically acceptable salt thereof, in combination with an effective amount of pridopidine, or a pharmaceutically acceptable salt thereof, for use in providing improvement in next day functioning of a subject with Huntington's disease. In embodiments, provided herein is a compound of Formula (I), or a pharmaceutically acceptable salt thereof, in combination with an effective amount of pridopidine, or a pharmaceutically acceptable salt thereof for the manufacture of a medicament for treatment of Huntington's disease.

Without wishing to be bound by any particular theory, symptoms of Huntington's disease can result from impaired Cl⁻ transport. See, e.g., Dargaei et al., PNAS 115 (7) E1618-E1626 (2018) (https://www.pnas.org/doi/full/10.1073/pnas.1716871115). KCC2 is a K⁺—Cl⁻ cotransporter and responsible for maintaining low Cl⁻ concentration in neurons of the central nervous system (CNS), essential for postsynaptic inhibition through GABA_{A} and glycine receptors. As a result of their KCC2 activation activity, the compounds of Formula (I), or pharmaceutically acceptable salts thereof, restore GABAergic function in patients diagnosed with Huntington's disease involving impaired Cl⁻ transport, thereby reducing, relieving, alleviating or eliminating certain symptoms.

Symptoms of Huntington's disease include motor, cognitive, and psychiatric disturbances. Motor disturbances can include hyperkinesia, chorea, hypokinesia, bradykinesia, dystonia, dysarthria, dysphagia, tics, ataxia, pyramidal signs such as the Babinski sign, difficulty walking, difficulty standing, frequent falls, gait abnormalities, and eye movement abnormalities. Behavioral symptoms can include poor attention, impulsivity, irritability, outbursts of anger, aggression, emotional blandness, apathy, loss of intuition, loss of creativity, depression, psychosis, cognitive decline, difficulty in organizing, multitasking and planning, and dementia. Other symptoms include weight loss, sleep disturbances, autonomic nervous system dysfunction (dysautonomia) and seizures.

In embodiments, the terms “effective amount” or “therapeutically effective amount” as applied to treating Huntington's disease refer to an amount of a compound, material, composition, medicament, or other material that is effective to achieve a particular pharmacological and/or physiologic effect in connection with symptoms of Huntington's disease described above.

In embodiments, effective amount refers to an amount which may be suitable to prevent a decline in or exacerbation of any one or more of the above symptoms, or, in embodiments, to improve any one or more of the above symptoms. In embodiments, effective amount refers to an amount which may be suitable to prevent a decline in two or more of the above symptoms, or, in embodiments, to improve two or more of the above symptoms. In embodiments, effective amount refers to an amount which may be suitable to prevent a decline in three or more of the above symptoms, or, in embodiments, to improve three or more of the above symptoms. In embodiments, an effective amount may be suitable to reduce either the extent or rate of decline in a subject's cognitive skills or functioning, and/or the effective amount may be suitable to delay the onset of such decline. Such effectiveness may be achieved, for example, by administering compositions described herein to an individual or to a population. In embodiments, the reduction, or delay of such a decline, or the improvement in an individual or population can be relative to a cohort, e.g., a control subject or a cohort population that has not received the treatment or been administered the composition or medicament. The terms “effective amount” and “therapeutically effective amount” are used interchangeably herein.

The dosage amount can vary according to a variety of factors such as subject-dependent variables (e.g., age, immune system, health, etc.), the disease or disorder being treated, as well as the route of administration and the pharmacokinetics of the agent being administered.

Many pharmaceutical products are administered as a fixed dose, at regular intervals, to achieve therapeutic efficacy. Duration of action is typically reflected by a drug's plasma half-life. Since efficacy is often dependent on sufficient exposure within the central nervous system administration of CNS drugs with a short half-life may require frequent maintenance dosing. CNS drugs with a long half-life may require less frequent maintenance dosing.

It should be understood that in the context of Formula (I) and other Formulas disclosed herein that unless otherwise indicated, the term “alkyl” includes both linear and branched chain alkyl groups. The prefix C_{p-q} in C_{p-q} alkyl and other terms (where p and q are integers) indicates the range of carbon atoms that are present in the group, for example C₁₋₃alkyl includes C₁alkyl (methyl), C₂alkyl (ethyl) and C₃alkyl (propyl as n-propyl and isopropyl).

The term “C_{p-q} alkoxy” comprises —O—C_{p-q} alkyl groups and —C_{p-q} alkyl groups where the O atom is within the alkyl chain, for example, —CH₂—O—CH₃.

The term “C_{p-q} alkenyl” includes both linear and branched chain alkyl groups containing at least two carbon atoms and at least one double carbon-carbon bond.

The term “C_{p-q} alkenyloxy” comprises —O—C_{p-q} alkenyl groups and —C_{p-q} alkenyl groups where the O atom is within the alkenyl chain.

The term “C_{p-q} alkynyl” includes both linear and branched chain alkyl groups containing at least two carbon atoms and at least one triple carbon-carbon bond.

The term “C_{p-q} alkynyloxy” comprises —O—C_{p-q} alkynyl groups and —C_{p-q} alkynyl groups where the O atoms is within the alkynyl chain.

C_{p-q} cycloalkyl refers to a cyclic non-aromatic group of p-q carbon atoms and no heteroatoms. For example, a 3 to 7 membered cycloalkyl refers to a ring containing 3 to 7 carbon atoms. Suitable C₃₋₇cycloalkyl rings include cyclopropyl, cyclobutyl, cyclopentyl and cyclohexyl.

Aryl is a 6 to 10 membered monocyclic or bicyclic aromatic ring containing no heteroatoms. Aryl includes phenyl.

Heterocycloalkyl is a monocyclic saturated or partially unsaturated, non-aromatic ring having, for example, 3 to 7 members, such as 3 to 6 members, 5 to 7 members such as 5 or 6 members, where at least one member and up to 4 members, particularly 1, 2 or 3 members of the ring are heteroatoms selected from N, O and S, and the remaining ring atoms are carbon atoms, in stable combinations known to those of skill in the art. Heterocycloalkyl ring nitrogen and sulphur atoms are optionally oxidised. Suitable heterocycloalkyl rings include morpholinyl, thiazolidinyl, homomorpholine, tetrahydropyranyl, pyrrolyl, thiomorpholinyl and tetrahydrofuranyl. In embodiments, when R⁷ is heterocycloalkyl, optionally two substituents on the same ring carbon together with the carbon to which they are attached form a 5 to 7 membered heterocycloalkyl ring, thereby creating a spirocyclic ring system. For example, in embodiments, R⁷ is morpholinyl and two substituents on the same ring carbon together form a tetrahydropyran.

Heteroaryl is a polyunsaturated, monocyclic 5 or 6 membered aromatic ring containing at least one and up to 3 heteroatoms, particularly, 1 or 2 heteroatoms selected from N, O and S, and the remaining ring atoms are carbon atoms. Heteroaryl ring nitrogen and sulphur atoms are optionally oxidised. Suitable heteroaryl rings include pyridinyl, isoxazolyl, oxadiazolyl, imidazolyl, pyrazinyl, oxazolyl, thiophenyl and thiazolyl.

The term “halo” is fluorine, chlorine or bromine.

The use of the dashed bond “ [symbol] ” in rings A of Formula (I) represents the fusion of the pyrimidine ring.

Where the term “optionally” is used, it is intended that the subsequent feature may or may not occur. As such, use of the term “optionally” includes instances where the feature is present, and also instances where the feature is not present. For example, a group “optionally substituted with 1, 2 or 3 —F substituents” includes group with and without an —F substituent.

The term “substituted” means that one or more hydrogens (for example 1 or 2 hydrogens, or alternatively 1 hydrogen) on the designated group is replaced by the indicated substituent(s) (for example 1, 2 or 3 substituents, or alternatively 1 or 2 substituents, or alternatively 1 substituent), provided that any atom(s) bearing a substituent maintains a permitted valency. Substituent combinations encompass only stable compounds and stable synthetic intermediates. “Stable” means that the relevant compound or intermediate is sufficiently robust to be isolated and have utility either as a synthetic intermediate or as an agent having potential therapeutic utility. If a group is not described as “substituted”, or “optionally substituted”, it is to be regarded as unsubstituted (i.e. that none of the hydrogens on the designated group have been replaced).

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich:Wiley-VCH/VHCA, 2002. In embodiments, a suitable pharmaceutically acceptable salt of a compound of the Formula (I) is, for example, a salt formed within the human or animal body after administration of a compound of the Formula (I), to said human or animal body.

In embodiments, a suitable pharmaceutically acceptable salt of a compound of Formula (I) is, for example, an acid addition salt. An acid addition salt of a compound of Formula (I) may be formed by bringing the compound into contact with a suitable inorganic or organic acid under conditions known to the skilled person. In embodiments, compounds described herein may form base addition salts. A base-addition salt of a compound of Formula (I) may be formed by bringing the compound into contact with a suitable inorganic or organic base under conditions known to the skilled person.

In embodiments, a compound according to Formula (I) may be provided as an acid addition salt, a zwitter ion hydrate, zwitter ion anhydrate, hydrochloride or hydrobromide salt, or in the form of the zwitter ion monohydrate. Acid addition salts, include but are not limited to, maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, pantothenic, p-amino-benzoic, glutamic, benzene sulfonic or theophylline acetic acid addition salts, as well as the 8-halotheophyllines, for example 8-bromo-theophylline. In embodiments, inorganic acid addition salts, including but not limited to, hydrochloric, hydrobromic, hydroiodic, sulfuric, sulfamic, phosphoric or nitric acid addition salts may be used.

In embodiments, there is provided a compound of Formula (I), or a pharmaceutically acceptable salt thereof. In embodiments, there is provided a compound of Formula (I). In embodiments there is provided a pharmaceutically acceptable salt of a compound of Formula (I).

Compounds and salts described herein may exist in solvated forms and unsolvated forms. For example, a solvated form may be a hydrated form, such as a hemi-hydrate, a monohydrate, a dihydrate, a trihydrate or an alternative quantity thereof. The description herein encompasses all such solvated and unsolvated forms of compounds of Formula (I), particularly to the extent that such forms possess KCC2 modulating activity, as for example measured using the tests described herein.

The following embodiments of moiety A may be applied to the description of the compounds of Formula (I), provided herein:
A is selected from:

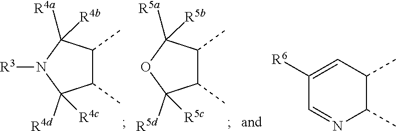

or a N-oxide thereof.

In embodiments, A is

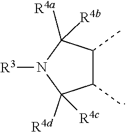

In embodiments, A is

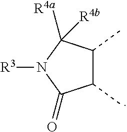

In embodiments, A is

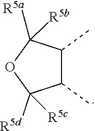

In embodiments, A is N

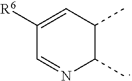

In embodiments, A is

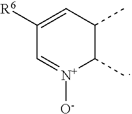

In embodiments, there is provided a compound of Formula (II):

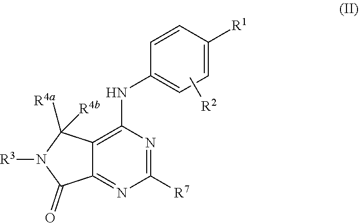

or a pharmaceutically acceptable salt thereof, wherein R¹, R², R³, R^{4a}, R^{4b} and R⁷ are as defined for Formula (I).

In embodiments, there is provided a compound of Formula (II) or a pharmaceutically acceptable salt thereof, wherein R¹, R², R³, R^{4a}, R^{4b} and R⁷ are as defined for Formula (I) and when R⁷ is morpholinyl, either:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃; heteroaryl is optionally substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and aryl is substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and R² is selected from —H; -halo; and —C₁₋₃alkyl optionally substituted with 1, 2 or 3 substituents selected from —F and —CF₃; or
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃; and heteroaryl is optionally substituted with 1 or 2 substituents selected from -halo, —C₁₋₃alkyl, —C₁₋₈alkoxy and —C₂₋₈alkynyloxy, wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; and R² is selected from -halo and —C₁₋₃alkyl optionally substituted with 1, 2 or 3 substituents selected from —F and —CF₃.

In embodiments, there is provided a compound of Formula (II) or a pharmaceutically acceptable salt thereof, wherein:
R¹ is selected from —C₂₋₆alkyl; —C₂₋₆alkoxy; C₃₋₇cycloalkyl; —O—C₃₋₇cycloalkyl; phenyl optionally substituted with 1 or 2 substituents selected from -halo, —C₁₋₃alkyl, —C₁₋₈alkoxy and —C₂₋₈alkynyloxy wherein —C₁₋₃ alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; —O— phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃;
R² is selected from —H, —F and —CH₃;
R³ is selected from —C₂₋₄alkynyl and —C₁₋₃alkyl optionally substituted with —NR⁸R⁹;
R^{4a} and R^{4b} are both —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1 or 2 substituents selected from —CN; —C₁₋₃alkyl optionally substituted with 1, 2 or 3 substituents selected from —F, —CF₃ and —OH; —C₁₋₃alkoxy; cyclopropyl; —C(O)OH; —C₁₋₃alkylene-NHC(O)C₁₋₆alkyl; —C₁₋₃alkylene-NHC(O)OC₁₋₆alkyl; or the heterocycloalkyl is optionally substituted with two substituents on the same ring carbon which together with the carbon atom to which they are attached form a 6 membered monocyclic heterocycloalkyl;
R⁸ and R⁹ are each independently selected from —C₁₋₆alkyl;
R¹⁰ is selected from —C₁₋₃alkyl;
R¹¹ is selected from —C₁₋₃alkyl optionally substituted with 1 or 2 substituents selected from —F and —C₁₋₃alkoxy; and —(CH₂)ₜR¹²;
R¹² is selected from a 5 or 6 membered heteroaryl, a 3 to 5 membered cycloalkyl or a 3 to 6 membered heterocycloalkyl;
n is 1 or 2.

In embodiments, there is provided a compound of Formula (II) or a pharmaceutically acceptable salt thereof, wherein:
R¹ is selected from —C₂₋₆alkyl; —C₂₋₆alkoxy; C₃₋₇cycloalkyl; —O—C₃₋₇cycloalkyl; phenyl substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl; —O-phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃; and R² is selected from —H, —F and —CH₃; or
R¹ is selected from —C₂₋₆alkyl; —C₂₋₆alkoxy; C₃₋₇cycloalkyl; —O—C₃₋₇cycloalkyl; unsubstituted phenyl; —O— phenyl optionally substituted with 1 or 2 -halo substituents; —O—CH₂-phenyl; and thiophenyl; wherein —C₂₋₆alkyl and —C₂₋₆alkoxy are optionally substituted with 1, 2 or 3 substituents selected from —F and —CF₃; and R² is selected from —F and —CH₃;
R³ is selected from —C₂₋₄alkynyl and —C₁₋₃alkyl optionally substituted with —NR⁸R⁹;
R^{4a} and R^{4b} are both —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1 or 2 substituents selected from —CN; —C₁₋₃alkyl optionally substituted with 1, 2 or 3 substituents selected from —F, —CF₃ and —OH; —C₁₋₃alkoxy; cyclopropyl; —C(O)OH; —C₁₋₃alkylene-NHC(O)C₁₋₆alkyl; —C₁₋₃alkylene-NHC(O)OC₁₋₆alkyl; or the heterocycloalkyl is optionally substituted with two substituents on the same ring carbon which together with the carbon atom to which they are attached form a 6 membered monocyclic heterocycloalkyl;
R⁸ and R⁹ are each independently selected from —C₁₋₆alkyl;
R¹⁰ is selected from —C₁₋₃alkyl;
R¹¹ is selected from —C₁₋₃alkyl optionally substituted with 1 or 2 substituents selected from —F and —C₁₋₃alkoxy; and —(CH₂)ₜR¹²;
R¹² is selected from a 5 or 6 membered heteroaryl, a 3 to 5 membered cycloalkyl or a 3 to 6 membered heterocycloalkyl; and
n is 1 or 2.

In embodiments, there is provided a compound of Formula (III):

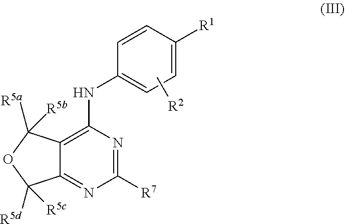

or a pharmaceutically acceptable salt thereof, wherein R¹, R², R^{5a}, R^{5b}, R^{5c}, R^{5d} and R⁷ are as defined for Formula (I).

In embodiments, there is provided a compound of Formula (III) or a pharmaceutically acceptable salt thereof, wherein:
R¹ is selected from C₃₋₇cycloalkyl and C₆₋₁₀aryl, wherein the aryl is optionally substituted with a —C₂₋₈alkoxy substituent wherein the alkoxy is optionally substituted with 1 or 2 —CF₃ substituents;
R² is —H;
R^{5a}, R^{5b}, R^{5c} and R^{5d} are each —H;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H.

In embodiments, there is provided a compound of Formula (III) or a pharmaceutically acceptable salt thereof, wherein:
R¹ is selected from C₃₋₇cycloalkyl and C₆₋₁₀aryl, wherein the aryl is optionally substituted with a —C₂₋₈alkoxy substituent wherein the alkoxy is optionally substituted with 1 or 2 —CF₃ substituents;
R² is —H;
R^{5a}, R^{5b}, R^{5c} and R^{5d} are each —H;
R⁷ is selected from a 5 to 7 membered monocyclic heterocycloalkyl and a 5 or 6 membered monocyclic heteroaryl, wherein the heterocycloalkyl and heteroaryl are optionally substituted with a substituent selected from —C₁₋₃alkyl optionally substituted with 1, 2 or 3 substituents selected from —F and —OH; and cyclopropyl.

In embodiments, there is provided a compound of Formula (IV):

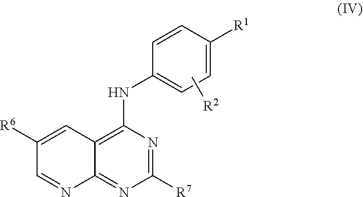

or a N-oxide or pharmaceutically acceptable salt thereof, wherein R¹, R², R⁶ and R⁷ are as defined for Formula (I).

In embodiments, there is provided a compound of Formula (IV), or a N-oxide or pharmaceutically acceptable salt thereof, wherein:
R¹ selected from C₃₋₇cycloalkyl optionally substituted with 1, 2 or 3 substituents selected from —F and —CF₃;
R² is selected from —H; -halo; and —C₁₋₃alkyl optionally substituted with 1, 2 or 3 substituents selected from —F and —CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl;
R⁸ and R⁹ are each independently selected from —H and —C₁₋₆alkyl;
R¹⁰ is —C₁₋₆alkyl;
R¹¹ is selected from —C₁₋₆alkyl optionally substituted with 1 or 2 substituents selected from —F and —C₁₋₃alkoxy; and —(CH₂)R¹²;
R¹² is a 5 or 6 membered heteroaryl, a 3 to 5 membered cycloalkyl or a 3 to 6 membered heterocycloalkyl;
n is 1, 2 or 3.

In embodiments, there is provided a compound of Formula (IV), or a N-oxide or pharmaceutically acceptable salt thereof, wherein:
R¹ selected from C₃₋₇cycloalkyl optionally substituted with 1, 2 or 3 substituents selected from —F and —CF₃;
R² is —H;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl;
R⁷ is selected from a 5 to 7 membered monocyclic heterocycloalkyl optionally substituted with 1, 2 or 3 groups selected from —C₁₋₆alkyl optionally substituted with 1, 2 or 3 substituents selected from —F, —CF₃ and —OH; and C₃₋₅cycloalkyl;
R¹ and R⁹ are each independently selected from —H and —C₁₋₆alkyl.

The following embodiments of moieties R¹, R², R³, R^{4a}, R^{4b}, R^{4c}, R^{4d}, R⁵, R^{6a}, R^{6b}, R⁷, R⁸, R⁹, R¹⁰, R¹¹, R¹², m and n may be applied, alone or in combination, to the description of the compounds of Formula (I) provided herein. The following embodiments of moieties R¹, R², R³, R^{4a}, R^{4b}, R⁷, R⁸, R⁹, R¹⁰, R¹¹, R¹², m and n may be applied, alone or in combination, to the description of the compounds of Formula (II) provided herein. The following embodiments of moieties R¹, R², R^{5a}, R^{5b}, R^{5c}, R^{5d}, R⁷, R⁸, R⁹, R¹⁰, R¹¹, R¹², m and n may be applied, alone or in combination, to the description of the compounds of Formula (III) provided herein. The following embodiments of moieties R¹, R², R⁶, R⁷, R⁸, R⁹, R¹⁰, R¹¹, R¹², m and n may be applied, alone or in combination, to the descriptions of the compounds of Formula (IV) provided herein.

R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₆alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein —O—(CH₂)ₘ—C₆₋₁₀aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl; and C₆₋₁₀aryl is substituted with 1 or 2 substituents selected from -halo, —C₁₋₃alkyl, —C₁₋₈alkoxy and —C₂₋₆alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl

In embodiments, R¹ is selected from C₂₋₆alkyl; C₂₋₆alkoxy; C₃₋₇cycloalkyl; —O—C₃₋₇cycloalkyl; C₆₋₁₀aryl; —O—(CH₂)ₘ—C₆₋₁₀aryl and thiophenyl; wherein alkyl, alkoxy and cycloalkyl are optionally substituted with 1, 2 or 3 substituents selected from —F and —CF₃ and wherein aryl is optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl optionally substituted with 1, 2 or 3 substituents selected from —F and —CF₃; C₂₋₄alkoxy; C₄₋₆cycloalkyl; —O—C₄₋₆cycloalkyl; phenyl; —O—(CH₂)ₘ-phenyl; and thiophenyl; wherein phenyl is optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from C₂₋₆alkyl optionally substituted with 1, 2 or 3 substituents selected from —F and —CF₃; C₂₋₄alkoxy; C₄₋₆cycloalkyl; —O—C₄₋₆cycloalkyl; phenyl; —O—(CH₂)ₘ-phenyl; and thiophenyl; wherein —O—(CH₂)ₘ-phenyl is optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl; and phenyl is substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃ and —NHC(O)O—C₁₋₆alkyl or two substituents together with the carbon to which they are attached form diazirinyl.

In embodiments, R¹ is selected from —CF₂CF₃; propyl; butyl; pentyl; propoxy; cyclobutyl; cyclohexyl; —O-cyclopentyl; thiophenyl; phenyl; —O-phenyl; —O—CH₂-phenyl; wherein phenyl is optionally substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇.

In embodiments, R¹ is selected from —CF₂CF₃; propyl; butyl; pentyl; propoxy; cyclobutyl; cyclohexyl; —O-cyclopentyl; thiophenyl; phenyl substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇; —O-phenyl; —O—CH₂-phenyl; wherein —O-phenyl and —O—CH₂-phenyl is optionally substituted with 1 or 2 substituents selected from —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇.

In embodiments, R¹ is cyclohexyl. In another embodiment, R¹ is phenyl substituted with —F, —Cl, —CH₃, —O—(CH₂)₅C≡CH, —O—(CH₂)₇, —O—(CH₂)₂C(N═N)(CH₂)₂C≡CH, —O—(CH₂)₂NHC(O)OC(CH₃)₃, —O—CH₂C≡CH, —O—(CH₂)₅CF₃ and —O—(CH₂)₇. In embodiments, R¹ is phenyl.

R² is selected from —H, -halo and —C₁₋₃alkyl optionally substituted with 1, 2 or 3 substituents selected from —F and —CF₃. In embodiments, R² is —H. In embodiments, R² is -halo. In embodiments, R² is —F. In embodiments, R² is —C₁₋₃alkyl. In embodiments, R² is methyl.

R³ is selected from —H; —C₁₋₆alkyl; —C₂₋₆alkenyl; —C₂₋₆alkynyl; —C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups, for example 1 or 2 groups, selected from —C₁₋₃alkyl optionally substituted with 1, 2 or 3 substituents selected from —F, —CF₃, —C(O)NR⁸R⁹ and —NR⁸R⁹.

In embodiments, R³ is selected from —H; —C₂₋₄alkynyl; —C₁₋₃alkyl optionally substituted with —C(O)NR⁸R⁹ or —NR⁸R⁹; and a 5 or 6 membered heterocycloalkyl optionally substituted with C₁₋₃alkyl.

In embodiments, R³ is selected from —H; —C₂₋₄alkynyl; —C₁₋₃alkyl optionally substituted with —C(O)NR⁸R⁹ or —NR⁸R⁹; and a 5 or 6 membered nitrogen containing heterocycloalkyl optionally substituted with C₁₋₃alkyl.

In embodiments, R³ is selected from —H; —C₂₋₄alkynyl; —C₁₋₃alkyl optionally substituted with —C(O)NR⁸R⁹ or —NR⁸R⁹; and piperidinyl optionally substituted with C₁₋₃alkyl.

In embodiments, R³ is selected from methyl, ethyl, i-propyl, —(CH₂)₂N(CH₃)₂, —(CH₂)₃N(CH₃)₂, —CH₂C≡CH, —CH₂C(O)N(CH₃)₂ and N-methylpiperidine. In embodiments, R³ is selected from ethyl, i-propyl, —(CH₂)₂N(CH₃)₂, —(CH₂)₃N(CH₃)₂, —CH₂C≡CH, —CH₂C(O)N(CH₃)₂ and N-methylpiperidine.

In embodiments, R³ is selected from —C₂₋₄alkynyl and —C₁₋₃alkyl optionally substituted with —NR⁸R⁹.

In embodiments, R³ is selected from ethyl, i-propyl, —(CH₂)₂N(CH₃)₂, —(CH₂)₃N(CH₃)₂ and —CH₂C≡CH.

In embodiments, R³ is i-propyl.

R^{4a} and R^{4b} are each independently selected from —H and —C₁₋₃alkyl optionally substituted with 1, 2 or 3 substituents selected from —F and —CF₃. In embodiments, R^{4a} is methyl and R^{4b} is —H. In embodiments, R^{4a} and R^{4b} are both —H.

R^{4c} and R^{4d} are each independently selected from —H and C₁₋₃ alkyl optionally substituted with 1, 2 or 3 substituents selected from —F and —CF₃; or R^{4c} and R^{4d} together with the carbon to which they are attached represent carbonyl. In embodiments, R^{4c} and R^{4d} together with the carbon to which they are attached represent carbonyl. In embodiments, R^{4c} and R^{4d} are each independently selected from —H and C₁₋₃ alkyl optionally substituted with 1, 2 or 3 substituents selected from —F and —CF₃. In embodiments, R^{4c} and R^{4d} are both —H or together with the carbon to which they are attached represent carbonyl. In embodiments, R^{4c} and R^{4d} are both —H.

R^{5a}, R^{5b}, R^{5c} and R^{5d} are each independently selected from —H and —C₁₋₃ alkyl optionally substituted with 1, 2 or 3 substituents selected from —F and —CF₃. In embodiments, R^{5a}, R^{5b}, R^{5c} and R^{5d} are each independently selected from —H and —C₁₋₃ alkyl optionally substituted with 1, 2 or 3 substituents selected from —F and —CF₃. In embodiments, R^{5a}, R^{5b}, R^{5c} and R^{5d} are each independently selected from —H and —C₁₋₃ alkyl. In embodiments, R^{5a} is methyl and R^{5b}, R^{5c} and R^{5d} are each —H. In embodiments, R^{5a}, R^{5b} and R^{5c} are each —H and R^{5d} is methyl. In embodiments, R^{5a}, R^{5b}, R^{5c} and R^{5d} each represent —H.

R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃alkyl. In embodiments, R⁶ is selected from —H; —Br; —NH₂; —CN; methoxy; ethyl; —C(O)OCH₃; —C(O)NH₂; —C(O)OH; and —NHC(O)CH₃.

R⁷ is selected from —NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl.

In embodiments, R⁷ is selected from NR¹⁰R¹¹; a 5 to 7 membered monocylic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl; and C₃₋₅cycloalkyl; or the heterocycloalkyl is optionally substituted with two substituents on the same ring carbon which together with the carbon atom to which they are attached form a 5 to 7 membered monocyclic heterocycloalkyl.

In embodiments, R⁷ is selected from NR¹⁰R¹¹; a 5 to 7 membered monocylic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1, 2 or 3 (for example, 1 or 2) groups selected from —CN, methyl, ethyl, propyl, cyclopropyl, methoxy, —CH₂CF₃, —CH₂OH, —CH₂CH₂OH, —C(O)OH, —(CH₂)₂NHC(O)CH₃ and —CH₂NHC(O)OC(CH₃)₃; or the heterocycloalkyl is optionally substituted with two substituents on the same ring carbon which together with the carbon atom to which they are attached form a 6 membered monocyclic heterocycloalkyl.

In embodiments, R⁷ is selected from NR¹⁰R¹¹ wherein R¹⁰ is selected from methyl, ethyl or propyl and R¹¹ is selected from ethyl, propyl, CH₂CHF₂, CH₂CH₂OCH₂CH₃ and —(CH₂)ₚR¹²; a 5 to 7 membered monocyclic heterocycloalkyl selected from morpholinyl, thiazolidinyl, tetrahydropyranyl, pyrrolyl, thiomorpholinyl and 3,4-dihydro-2H-pyranyl; a 5 or 6 membered monocyclic heteroaryl selected from pyridinyl, dihydropyranyl, oxazolyl, imidazolyl and thiazolyl; wherein the heterocycloalkyl and heteroaryl are optionally substituted with 1 or 2 groups selected from —CN, methyl, ethyl, propyl, cyclopropyl, methoxy, —CH₂CF₃, —CH₂OH, —CH₂CH₂OH, —C(O)OH, —(CH₂)₂NHC(O)CH₃ and —CH₂NHC(O)OC(CH₃)₃; or the heterocycloalkyl is optionally substituted with two substituents on the same ring carbon which together with the carbon atom to which they are attached form tetrahydropyranyl.

In embodiments, R⁷ is selected from NR¹⁰R¹¹ wherein R¹⁰ is selected from methyl, ethyl or propyl and R¹¹ is selected from ethyl, propyl, CH₂CHF₂, CH₂CH₂OCH₂CH₃ and —(CH₂)ₙR¹².

In embodiments, R⁷ is selected from NR¹⁰R¹¹ wherein R¹⁰ is selected from methyl, ethyl or propyl; R¹¹ is selected from ethyl, propyl, CH₂CHF₂, CH₂CH₂OCH₂CH₃ and —(CH₂)ₙR¹²; n is 1 or 2; and R¹² is selected from isoxazolyl, oxadiazolyl, cyclopropyl, pyrazinyl, tetrahydrofuranyl and pyridinyl.

In embodiments, R⁷ is selected from a 5 to 7 membered monocyclic heterocycloalkyl optionally substituted with 1, 2 or 3 (for example, 1 or 2) groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —C₁₋₃alkylene-NHC(O)C₁₋₆alkyl; —C₁₋₃alkylene-NHC(O)OC₁₋₆alkyl and C₃₋₅cycloalkyl; or the heterocycloakyl is optionally substituted with two substituents on the same ring carbon which together with the carbon atom to which they are attached form a 5 to 7 membered monocyclic heterocycloalkyl.

In embodiments, R⁷ is a 5 to 7 membered monocyclic heterocycloalkyl selected from morpholinyl, thiazolidinyl, tetrahydropyranyl, pyrrolyl, thiomorpholinyl and 3,4-dihydro-2H-pyranyl wherein the heterocycloalkyl is optionally substituted with 1 or 2 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and —CF₃; —C(O)OH; —CH₂NHC(O)CH₃; —CH₂NHC(O)OC(CH₃)₃; and C₃₋₅cycloalkyl; or the heterocycloalkyl is optionally substituted with two substituents on the same ring carbon which together with the carbon atom to which they are attached form a 6 membered monocyclic heterocycloalkyl.

In embodiments, R⁷ is a 5 to 7 membered monocyclic heterocyclalkyl optionally substituted with 1 or 2 substituents selected from methyl, ethyl, propyl, cyclopropyl, —CH₂CH₂OH, —CH₂OH, —C(O)OH, —CH₂CF₃, and —CH₂NHC(O)OC(CH₃)₃; or the heterocycloalkyl is optionally substituted with two substituents on the same ring carbon which together with the carbon atom to which they are attached form tetrahydropyran.

In embodiments, R⁷ is morpholinyl optionally substituted with 1 or 2 substituents selected from methyl, ethyl, propyl, cyclopropyl, —CH₂CH₂OH, —CH₂OH, —C(O)OH, —CH₂CF₃, and —CH₂NHC(O)OC(CH₃)₃; or optionally substituted with two substituents on the same ring carbon which together with the carbon atom to which they are attached form tetrahydropyran (i.e. R⁷ becomes a spirocyclic group).

In embodiments, R⁷ is 2-methylmorpholin-4-yl.

R⁸ is selected from —H and —C₁₋₆alkyl. In embodiments, R⁸ is selected from —H and —C₁₋₃alkyl. In embodiments, R⁸ is —H. In embodiments, R⁸ is —C₁₋₃alkyl. In embodiments, R⁸ is methyl.

R⁹ is selected from —H and —C₁₋₆alkyl. In embodiments, R⁹ is selected from —H and —C₁₋₃alkyl. In embodiments, R⁹ is —H. In embodiments, R⁹ is —C₁₋₃alkyl. In embodiments, R⁹ is methyl.

R¹⁰ is —C₁₋₆alkyl. In embodiments, R¹⁰ is —C₁₋₃alkyl. In embodiments, R¹⁰ is methyl. In another embodiment, R¹⁰ is ethyl. In embodiments, R¹⁰ is propyl.

R¹¹ is selected from —C₁₋₆alkyl optionally substituted with 1 or 2 substituents selected from —F and —C₁₋₃-alkoxy; or —(CH₂)ₙR¹². In embodiments, R¹¹ is selected from —C₁₋₆alkyl optionally substituted with 1 or 2 substituents selected from —F and ethoxy. In embodiments, R¹¹ is selected from ethyl, propyl, CH₂CHF₂, CH₂CH₂OCH₂CH₃ and —(CH₂)ₙR¹². In embodiments, R¹¹ is selected from —(CH₂)ₙR¹².

R¹² is selected from a 5 or 6 membered heteroaryl, a 3 to 5 membered cycloalkyl or a 3 to 6 membered heterocycloalkyl. In embodiments, R¹² is selected from isoxazolyl, oxadiazolyl, cyclopropyl, pyrazinyl, tetrahydrofuranyl and pyridinyl.

m is 0 or 1. In embodiments, m is 0. In embodiments, m is 1.

n is 1, 2 or 3. In embodiments, n is 1 or 2. In embodiments, n is 1. In embodiments, n is 2. In embodiments, n is 3.

In embodiments, the compound of Formula (I) is selected from:
2-(diethylamino)-6-(propan-2-yl)-4-{[4-(propan-2-yl)phenyl]amino}-5,6-dihydro-7H-pyrrolo[3,4-d]pyrimidin-7-one;
4-[(4-cyclohexylphenyl)amino]-2-(2-cyclopropylmorpholin-4-yl)-6-(propan-2-yl)-5,6-dihydro-7H-pyrrolo[3,4-d]pyrimidin-7-one;
6-(propan-2-yl)-4-{[4-(propan-2-yl)phenyl]amino}-2-(1,3-thiazolidin-3-yl)-5,6-dihydro-7H-pyrrolo[3,4-d]pyrimidin-7-one;
2-[(2R,6S)-2,6-dimethylmorpholin-4-yl]-6-(propan-2-yl)-4-{[4-(propan-2-yl)phenyl]amino}-5,6-dihydro-7H-pyrrolo[3,4-d]pyrimidin-7-one;
6-(propan-2-yl)-4-{[4-(propan-2-yl)phenyl]amino}-2-(thiomorpholin-4-yl)-5,6-dihydro-7H-pyrrolo[3,4-d]pyrimidin-7-one;
2-[(2S)-2-methylmorpholin-4-yl]-6-(propan-2-yl)-4-{[4-(propan-2-yl)phenyl]amino}-5,6-dihydro-7H-pyrrolo[3,4-d]pyrimidin-7-one;
2-[(2R)-2-methylmorpholin-4-yl]-6-(propan-2-yl)-4-{[4-(propan-2-yl)phenyl]amino}-5,6-dihydro-7H-pyrrolo[3,4-d]pyrimidin-7-one;
2-[(2S,6S)-2,6-dimethylmorpholin-4-yl]-6-(propan-2-yl)-4-{[4-(propan-2-yl)phenyl]amino}-5,6-dihydro-7H-pyrrolo[3,4-d]pyrimidin-7-one;
2-(3-methylmorpholin-4-yl)-6-(propan-2-yl)-4-{[4-(propan-2-yl)phenyl]amino}-5,6-dihydro-7H-pyrrolo[3,4-d]pyrimidin-7-one;
2-(2-cyclopropylmorpholin-4-yl)-6-(propan-2-yl)-4-{[4-(propan-2-yl)phenyl]amino}-5,6-dihydro-7H-pyrrolo[3,4-d]pyrimidin-7-one;
4-[(4-cyclohexylphenyl)amino]-2-(morpholin-4-yl)-6-(propan-2-yl)-5,6-dihydro-7H-pyrrolo[3,4-d]pyrimidin-7-one;
4-[(4-cyclohexylphenyl)amino]-2-(2-methylmorpholin-4-yl)-6-(propan-2-yl)-5,6-dihydro-7H-pyrrolo[3,4-d]pyrimidin-7-one;
4-[(4-cyclohexylphenyl)amino]-2-[(2R)-2-methylmorpholin-4-yl]-6-(propan-2-yl)-5,6-dihydro-7H-pyrrolo[3,4-d]pyrimidin-7-one;
4-[(4-cyclohexylphenyl)amino]-2-((2R)-cyclopropylmorpholin-4-yl)-6-(propan-2-yl)-5,6-dihydro-7H-pyrrolo[3,4-d]pyrimidin-7-one;
4-[(4-cyclohexylphenyl)amino]-2-((2S)-cyclopropylmorpholin-4-yl)-6-(propan-2-yl)-5,6-dihydro-7H-pyrrolo[3,4-d]pyrimidin-7-one;
4-[(4-cyclohexylphenyl)amino]-6-(propan-2-yl)-2-[2-(2,2,2-trifluoroethyl)morpholin-4-yl]-5,6-dihydro-7H-pyrrolo[3,4-d]pyrimidin-7-one;
tert-butyl {[(2R)-4-{4-[(4-cyclohexylphenyl)amino]-7-oxo-6-(propan-2-yl)-6,7-dihydro-5H-pyrrolo[3,4-d]pyrimidin-2-yl}morpholin-2-yl]methyl}carbamate;
4-[(4-cyclohexylphenyl)amino]-6-(propan-2-yl)-2-[2-(propan-2-yl)morpholin-4-yl]-5,6-dihydro-7H-pyrrolo[3,4-d]pyrimidin-7-one;
4-[(4-cyclohexylphenyl)amino]-6-(propan-2-yl)-2-(1,3-thiazolidin-3-yl)-5,6-dihydro-7H-pyrrolo[3,4-d]pyrimidin-7-one;
4-[(4-cyclohexylphenyl)amino]-2-[(2-ethoxyethyl)(methyl)amino]-6-(propan-2-yl)-5,6-dihydro-7H-pyrrolo[3,4-d]pyrimidin-7-one;
4-[(4-cyclohexylphenyl)amino]-2-(2-ethylmorpholin-4-yl)-6-(propan-2-yl)-5,6-dihydro-7H-pyrrolo[3,4-d]pyrimidin-7-one;
4-[(4-cyclohexylphenyl)amino]-2-{methyl[(1,2-oxazol-3-yl)methyl]amino}-6-(propan-2-yl)-5,6-dihydro-7H-pyrrolo[3,4-d]pyrimidin-7-one;
4-[(4-cyclohexylphenyl)amino]-2-{methyl[2-(1,2,4-oxadiazol-3-yl)ethyl]amino}-6-(propan-2-yl)-5,6-dihydro-7H-pyrrolo[3,4-d]pyrimidin-7-one;
4-[(4-cyclohexylphenyl)amino]-2-(1,4-oxazepan-4-yl)-6-(propan-2-yl)-5,6-dihydro-7H-pyrrolo[3,4-d]pyrimidin-7-one;
4-[(4-cyclohexylphenyl)amino]-2-(1,9-dioxa-4-azaspiro[5.5]undecan-4-yl)-6-(propan-2-yl)-5,6-dihydro-7H-pyrrolo[3,4-d]pyrimidin-7-one;
4-[(4-cyclohexylphenyl)amino]-2-(3-methoxypyrrolidin-1-yl)-6-(propan-2-yl)-5,6-dihydro-7H-pyrrolo[3,4-d]pyrimidin-7-one;
4-[(4-cyclohexylphenyl)amino]-2-[2-(2-hydroxyethyl)morpholin-4-yl]-6-(propan-2-yl)-5,6-dihydro-7H-pyrrolo[3,4-d]pyrimidin-7-one;
4-[(4-cyclohexylphenyl)amino]-2-(dipropylamino)-6-(propan-2-yl)-5,6-dihydro-7H-pyrrolo[3,4-d]pyrimidin-7-one;
4-[(4-cyclohexylphenyl)amino]-2-[(cyclopropylmethyl)(methyl)amino]-6-(propan-2-yl)-5,6-dihydro-7H-pyrrolo[3,4-d]pyrimidin-7-one;
4-[(4-cyclohexylphenyl)amino]-2-[2-(hydroxymethyl)morpholin-4-yl]-6-(propan-2-yl)-5,6-dihydro-7H-pyrrolo[3,4-d]pyrimidin-7-one;
4-[(4-cyclohexylphenyl)amino]-2-[3-(hydroxymethyl)morpholin-4-yl]-6-(propan-2-yl)-5,6-dihydro-7H-pyrrolo[3,4-d]pyrimidin-7-one;
4-[(4-cyclohexylphenyl)amino]-2-{methyl[(pyrazin-2-yl)methyl]amino}-6-(propan-2-yl)-5,6-dihydro-7H-pyrrolo[3,4-d]pyrimidin-7-one;
4-[(4-cyclohexylphenyl)amino]-2-(diethylamino)-6-(propan-2-yl)-5,6-dihydro-7H-pyrrolo[3,4-d]pyrimidin-7-one;
4-[(4-cyclohexylphenyl)amino]-2-{methyl[(oxolan-2-yl)methyl]amino}-6-(propan-2-yl)-5,6-dihydro-7H-pyrrolo[3,4-d]pyrimidin-7-one;
4-[(4-cyclohexylphenyl)amino]-2-[(2,2-difluoroethyl)(methyl)amino]-6-(propan-2-yl)-5,6-dihydro-7H-pyrrolo[3,4-d]pyrimidin-7-one;
4-[(4-cyclohexylphenyl)amino]-2-{methyl[2-(pyridin-2-yl)ethyl]amino}-6-(propan-2-yl)-5,6-dihydro-7H-pyrrolo[3,4-d]pyrimidin-7-one;
(3S)-4-{4-[(4-cyclohexylphenyl)amino]-7-oxo-6-(propan-2-yl)-6,7-dihydro-5H-pyrrolo[3,4-d]pyrimidin-2-yl}morpholine-3-carboxylic acid;
N-[2-(4-{4-[(4-cyclohexylphenyl)amino]-7-oxo-6-(propan-2-yl)-6,7-dihydro-5H-pyrrolo[3,4-d]pyrimidin-2-yl}morpholin-2-yl)ethyl]acetamide;
6-(propan-2-yl)-4-{[4-(propan-2-yl)phenyl]amino}-2-(pyridin-4-yl)-5,6-dihydro-7H-pyrrolo[3,4-d]pyrimidin-7-one;
6-isopropyl-4-((4-isopropylphenyl)amino)-2-(pyridin-4-yl)-5,6-dihydro-7H-pyrrolo[3,4-d]pyrimidin-7-one (Compound B);
4-{4-[(4-cyclohexylphenyl)amino]-7-oxo-6-(propan-2-yl)-6,7-dihydro-5H-pyrrolo[3,4-d]pyrimidin-2-yl}pyridine-2-carbonitrile;
4-[(4-cyclohexylphenyl)amino]-2-(2-cyclopropylpyridin-4-yl)-6-(propan-2-yl)-5,6-dihydro-7H-pyrrolo[3,4-d]pyrimidin-7-one;
4-[(4-cyclohexylphenyl)amino]-2-(2-methoxypyridin-4-yl)-6-(propan-2-yl)-5,6-dihydro-7H-pyrrolo[3,4-d]pyrimidin-7-one;
4-[(4-cyclohexylphenyl)amino]-2-(2-methylpyridin-4-yl)-6-(propan-2-yl)-5,6-dihydro-7H-pyrrolo[3,4-d]pyrimidin-7-one (Compound C);
4-[(4-cyclohexylphenyl)amino]-2-(3,6-dihydro-2H-pyran-4-yl)-6-(propan-2-yl)-5,6-dihydro-7H-pyrrolo[3,4-d]pyrimidin-7-one;
4-[(4-cyclohexylphenyl)amino]-6-(propan-2-yl)-2-(pyridin-4-yl)-5,6-dihydro-7H-pyrrolo[3,4-d]pyrimidin-7-one;
4-[(4-cyclohexylphenyl)amino]-2-(1-methyl-1H-pyrazol-4-yl)-6-(propan-2-yl)-5,6-dihydro-7H-pyrrolo[3,4-d]pyrimidin-7-one;
4-[(4-cyclohexylphenyl)amino]-2-(1,3-oxazol-5-yl)-6-(propan-2-yl)-5,6-dihydro-7H-pyrrolo[3,4-d]pyrimidin-7-one;
4-[(4-cyclohexylphenyl)amino]-6-(propan-2-yl)-2-(1,3-thiazol-5-yl)-5,6-dihydro-7H-pyrrolo[3,4-d]pyrimidin-7-one;
2-(3,6-dihydro-2H-pyran-4-yl)-6-(propan-2-yl)-4-{[4-(propan-2-yl)phenyl]amino}-5,6-dihydro-7H-pyrrolo[3,4-d]pyrimidin-7-one;
4-{[4-(4-fluorophenoxy)phenyl]amino}-2-[(2R)-2-methylmorpholin-4-yl]-6-(propan-2-yl)-5,6-dihydro-7H-pyrrolo[3,4-d]pyrimidin-7-one;
2-(2-cyclopropylmorpholin-4-yl)-4-({4′-[(hept-6-yn-1-yl)oxy][1,1′-biphenyl]-4-yl}amino)-6-(propan-2-yl)-5,6-dihydro-7H-pyrrolo[3,4-d]pyrimidin-7-one;
2-(2-cyclopropylmorpholin-4-yl)-4-{[4′-(heptyloxy)[1,1′-biphenyl]-4-yl]amino}-6-(propan-2-yl)-5,6-dihydro-7H-pyrrolo[3,4-d]pyrimidin-7-one;
4-[(4′-{2-[3-(but-3-yn-1-yl)-3H-diaziren-3-yl]ethoxy}[1,1′-biphenyl]-4-yl)amino]-2-(2-cyclopropyl-morpholin-4-yl)-6-(propan-2-yl)-5,6-dihydro-7H-pyrrolo[3,4-d]pyrimidin-7-one;
2-[(2R)-2-methylmorpholin-4-yl]-4-[(4-pentylphenyl)amino]-6-(propan-2-yl)-5,6-dihydro-7H-pyrrolo[3,4-d]pyrimidin-7-one;
4-{[4-(butan-2-yl)phenyl]amino}-2-[(2R)-2-methylmorpholin-4-yl]-6-(propan-2-yl)-5,6-dihydro-7H-pyrrolo[3,4-d]pyrimidin-7-one;
4-{[4-(benzyloxy)phenyl]amino}-2-[(2R)-2-methylmorpholin-4-yl]-6-(propan-2-yl)-5,6-dihydro-7H-pyrrolo[3,4-d]pyrimidin-7-one;
2-(2-cyclopropylmorpholin-4-yl)-4-{[4-(pentafluoroethyl)phenyl]amino}-6-(propan-2-yl)-5,6-dihydro-7H-pyrrolo[3,4-d]pyrimidin-7-one;
2-(2-cyclopropylmorpholin-4-yl)-6-(propan-2-yl)-4-[(4-propylphenyl)amino]-5,6-dihydro-7H-pyrrolo[3,4-d]pyrimidin-7-one;
2-[(2R)-2-methylmorpholin-4-yl]-6-(propan-2-yl)-4-[(4-propylphenyl)amino]-5,6-dihydro-7H-pyrrolo[3,4-d]pyrimidin-7-one;
2-[(2R)-2-methylmorpholin-4-yl]-4-{[4-(pentafluoroethyl)phenyl]amino}-6-(propan-2-yl)-5,6-dihydro-7H-pyrrolo[3,4-d]pyrimidin-7-one;
2-(2-cyclopropylmorpholin-4-yl)-6-(propan-2-yl)-4-({4-[(propan-2-yl)oxy]phenyl}amino)-5,6-dihydro-7H-pyrrolo[3,4-d]pyrimidin-7-one;
4-[(4-cyclobutylphenyl)amino]-2-(morpholin-4-yl)-6-(propan-2-yl)-5,6-dihydro-7H-pyrrolo[3,4-d]pyrimidin-7-one;
4-{[4-(cyclopentyloxy)phenyl]amino}-2-[(2R)-2-methylmorpholin-4-yl]-6-(propan-2-yl)-5,6-dihydro-7H-pyrrolo[3,4-d]pyrimidin-7-one;
2-[(2R)-2-methylmorpholin-4-yl]-6-(propan-2-yl)-4-{[4-(2,2,2-trifluoroethyl)phenyl]amino}-5,6-dihydro-7H-pyrrolo[3,4-d]pyrimidin-7-one;
tert-butyl {2-[(4′-{[2-(2-cyclopropylmorpholin-4-yl)-7-oxo-6-(propan-2-yl)-6,7-dihydro-5H-pyrrolo[3,4-d]pyrimidin-4-yl]amino}[1,1′-biphenyl]-4-yl)oxy]ethyl}carbamate;
6-ethyl-2-[(2R)-2-methylmorpholin-4-yl]-4-{[4-(propan-2-yl)phenyl]amino}-5,6-dihydro-7H-pyrrolo[3,4-d]pyrimidin-7-one;
4-[(4-cyclohexylphenyl)amino]-6-ethyl-2-[(2R)-2-methylmorpholin-4-yl]-5,6-dihydro-7H-pyrrolo[3,4-d]pyrimidin-7-one;
tert-butyl {2-[(4′-{[2-(morpholin-4-yl)-7-oxo-6-(propan-2-yl)-6,7-dihydro-5H-pyrrolo[3,4-d]pyrimidin-4-yl]amino}[1,1′-biphenyl]-4-yl)oxy]ethyl}carbamate;
4-[(4′-{2-[3-(but-3-yn-1-yl)-3H-diaziren-3-yl]ethoxy}[1,1′-biphenyl]-4-yl)amino]-2-(morpholin-4-yl)-6-(propan-2-yl)-5,6-dihydro-7H-pyrrolo[3,4-d]pyrimidin-7-one;
2-(morpholin-4-yl)-6-(propan-2-yl)-4-({4′-[(prop-2-yn-1-yl)oxy][1,1′-biphenyl]-4-yl}amino)-5,6-dihydro-7H-pyrrolo[3,4-d]pyrimidin-7-one;
4-[(4-cyclohexylphenyl)amino]-6-[3-(dimethylamino)propyl]-2-[(2R)-2-methylmorpholin-4-yl]-5,6-dihydro-7H-pyrrolo[3,4-d]pyrimidin-7-one;
4-[(4-cyclohexylphenyl)amino]-6-[2-(dimethylamino)ethyl]-2-[(2R)-2-methylmorpholin-4-yl]-5,6-dihydro-7H-pyrrolo[3,4-d]pyrimidin-7-one;
4-[(4-cyclobutylphenyl)amino]-6-[3-(dimethylamino)propyl]-2-[(2R)-2-methylmorpholin-4-yl]-5,6-dihydro-7H-pyrrolo[3,4-d]pyrimidin-7-one;
4-[(4-cyclobutylphenyl)amino]-6-[2-(dimethylamino)ethyl]-2-[(2R)-2-methylmorpholin-4-yl]-5,6-dihydro-7H-pyrrolo[3,4-d]pyrimidin-7-one;
2-(morpholin-4-yl)-4-{[4-(propan-2-yl)phenyl]amino}-6-(prop-2-yn-1-yl)-5,6-dihydro-7H-pyrrolo[3,4-d]pyrimidin-7-one;
4-[(4-cyclohexylphenyl)amino]-2-(oxan-4-yl)-6-(propan-2-yl)-5,6-dihydro-7H-pyrrolo[3,4-d]pyrimidin-7-one;
4-[(4-cyclohexylphenyl)amino]-2-(1H-imidazol-1-yl)-6-(propan-2-yl)-5,6-dihydro-7H-pyrrolo[3,4-d]pyrimidin-7-one;
2-(3,6-dihydro-2H-pyran-4-yl)-4-[(2′-methyl[1,1′-biphenyl]-4-yl)amino]-6-(propan-2-yl)-5,6-dihydro-7H-pyrrolo[3,4-d]pyrimidin-7-one;
4-[(4′-{2-[3-(but-3-yn-1-yl)-3H-diaziren-3-yl]ethoxy}[1,1′-biphenyl]-4-yl)amino]-2-(3,6-dihydro-2H-pyran-4-yl)-6-(propan-2-yl)-5,6-dihydro-7H-pyrrolo[3,4-d]pyrimidin-7-one;
2-(3,6-dihydro-2H-pyran-4-yl)-4-[(2-fluoro[1,1′-biphenyl]-4-yl)amino]-6-(propan-2-yl)-5,6-dihydro-7H-pyrrolo[3,4-d]pyrimidin-7-one;
2-(morpholin-4-yl)-4-{[4-(pentafluoroethyl)phenyl]amino}-6-(propan-2-yl)-5,6-dihydro-7H-pyrrolo[3,4-d]pyrimidin-7-one;
4-[(2-fluoro[1,1′-biphenyl]-4-yl)amino]-2-(morpholin-4-yl)-6-(propan-2-yl)-5,6-dihydro-7H-pyrrolo[3,4-d]pyrimidin-7-one;
4-[(3′,4′-dichloro[1,1′-biphenyl]-4-yl)amino]-2-(morpholin-4-yl)-6-(propan-2-yl)-5,6-dihydro-7H-pyrrolo[3,4-d]pyrimidin-7-one;
2-(morpholin-4-yl)-6-(propan-2-yl)-4-{[4-(propan-2-yl)phenyl]amino}-5,6-dihydro-7H-pyrrolo[3,4-d]pyrimidin-7-one;
4-[(4-tert-butylphenyl)amino]-2-(morpholin-4-yl)-6-(propan-2-yl)-5,6-dihydro-7H-pyrrolo[3,4-d]pyrimidin-7-one;
4-[(2-methyl[1,1′-biphenyl]-4-yl)amino]-2-(morpholin-4-yl)-6-(propan-2-yl)-5,6-dihydro-7H-pyrrolo[3,4-d]pyrimidin-7-one;
4-[(4′-chloro[1,1′-biphenyl]-4-yl)amino]-2-(morpholin-4-yl)-6-(propan-2-yl)-5,6-dihydro-7H-pyrrolo[3,4-d]pyrimidin-7-one;
N-(4-cyclohexylphenyl)-2-[(2R)-2-methylmorpholin-4-yl]-5,7-dihydrofuro[3,4-d]pyrimidin-4-amine;
N-(4-cyclobutylphenyl)-2-(3,6-dihydro-2H-pyran-4-yl)-5,7-dihydrofuro[3,4-d]pyrimidin-4-amine;
N-(4-cyclohexylphenyl)-2-(2-cyclopropylmorpholin-4-yl)-5,7-dihydrofuro[3,4-d]pyrimidin-4-amine;
2-(2-cyclopropylmorpholin-4-yl)-N-[4′-(heptyloxy)[1,1′-biphenyl]-4-yl]-5,7-dihydrofuro[3,4-d]pyrimidin-4-amine;
2-[(2R)-2-methylmorpholin-4-yl]-N-{4′-[(6,6,6-trifluorohexyl)oxy][1,1′-biphenyl]-4-yl}-5,7-dihydrofuro[3,4-d]pyrimidin-4-amine;
N-(4-cyclohexylphenyl)-2-(2-methylpyridin-4-yl)-5,7-dihydrofuro[3,4-d]pyrimidin-4-amine;
N-(4-cyclohexylphenyl)-2-[(2R)-2-methylmorpholin-4-yl]pyrido[2,3-d]pyrimidin-4-amine (Compound D);
6-bromo-N-(4-cyclohexylphenyl)-2-[(2R)-2-methylmorpholin-4-yl]pyrido[2,3-d]pyrimidin-4-amine;
N-(4-cyclohexylphenyl)-2-(3,6-dihydro-2H-pyran-4-yl)pyrido[2,3-d]pyrimidin-4-amine;
N-(4-cyclohexylphenyl)-2-[(2R)-2-methylmorpholin-4-yl]-8-oxo-8lambda-5˜-pyrido[2,3-d]pyrimidin-4-amine;
N-(4-cyclohexylphenyl)-6-ethyl-2-[(2R)-2-methylmorpholin-4-yl]pyrido[2,3-d]pyrimidin-4-amine;
4-[(4-cyclohexylphenyl)amino]-2-[(2R)-2-methylmorpholin-4-yl]pyrido[2,3-d]pyrimidine-6-carbonitrile;
methyl 4-[(4-cyclohexylphenyl)amino]-2-[(2R)-2-methylmorpholin-4-yl]pyrido[2,3-d]pyrimidine-6-carboxylate;
methyl (R)-4-((4-cyclohexylphenyl)amino)-2-(2-methylmorpholino)pyrido[2,3-d]pyrimidine-6-carboxylate (Compound E);
4-[(4-cyclohexylphenyl)amino]-2-[(2R)-2-methylmorpholin-4-yl]pyrido[2,3-d]pyrimidine-6-carboxylic acid;
4-[(4-cyclohexylphenyl)amino]-2-[(2R)-2-methylmorpholin-4-yl]pyrido[2,3-d]pyrimidine-6-carboxamide;
4-[(4-cyclohexylphenyl)amino]-2-(2-cyclopropylmorpholin-4-yl)pyrido[2,3-d]pyrimidine-6-carboxamide;
N-(4-cyclohexylphenyl)-6-methoxy-2-[(2R)-2-methylmorpholin-4-yl]pyrido[2,3-d]pyrimidin-4-amine;
(R)-N-(4-cyclohexylphenyl)-6-methoxy-2-(2-methylmorpholino)pyrido[2,3-d]pyrimidin-4-amine (Compound F);
N-{4-[(4-cyclohexylphenyl)amino]-2-[(2R)-2-methylmorpholin-4-yl]pyrido[2,3-d]pyrimidin-6-yl}acetamide;
N-4-(4-cyclohexylphenyl)-2-[(2R)-2-methylmorpholin-4-yl]pyrido[2,3-d]pyrimidine-4,6-diamine;
N-(4-cyclohexylphenyl)-2-(morpholin-4-yl)-6-(propan-2-yl)-6,7-dihydro-5H-pyrrolo[3,4-d]pyrimidin-4-amine;
N-(4-cyclohexylphenyl)-2-[(2R)-2-methylmorpholin-4-yl]-6-(propan-2-yl)-6,7-dihydro-5H-pyrrolo[3,4-d]pyrimidin-4-amine;
2-{4-[(4-cyclohexylphenyl)amino]-2-(3,6-dihydro-2H-pyran-4-yl)-5,7-dihydro-6H-pyrrolo[3,4-d]pyrimidin-6-yl}-N,N-dimethylacetamide (Compound G);
N-(4-cyclohexylphenyl)-2-(2-cyclopropylmorpholin-4-yl)-6-(1-methylpiperidin-4-yl)-6,7-dihydro-5H-pyrrolo[3,4-d]pyrimidin-4-amine;
2-(morpholin-4-yl)-6-(propan-2-yl)-4-{[4-(thiophen-2-yl)phenyl]amino}-5,6-dihydro-7H-pyrrolo[3,4-d]pyrimidin-7-one;
2-(morpholin-4-yl)-6-(propan-2-yl)-4-{[4-(thiophen-3-yl)phenyl]amino}-5,6-dihydro-7H-pyrrolo[3,4-d]pyrimidin-7-one;

and pharmaceutically acceptable salts thereof.

In embodiments, the compound of Formula (I) is

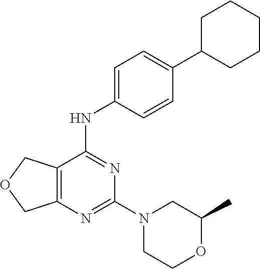 [SMILES: C[C@@H]1CN(c2nc3c(c(Nc4ccc(C5CCCCC5)cc4)n2)COC3)CCO1]

known as (R)-N-(4-cyclohexylphenyl)-2-(2-methylmorpholino)-5,7-dihydrofuro[3,4-d]pyrimidin-4-amine (also referred herein to as N-(4-cyclohexylphenyl)-2-[(2R)-2-methylmorpholin-4-yl]-5,7-dihydrofuro[3,4-d]pyrimidin-4-amine, or also referred to herein as Compound A or CmpA), or a pharmaceutically acceptable salt thereof.

In embodiments, the compound of Formula (I) is Compound B:

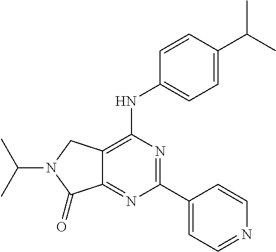 [SMILES: CC(C)c1ccc(Nc2nc(-c3ccncc3)nc3c2CN(C(C)C)C3=O)cc1]

6-isopropyl-4-((4-isopropylphenyl)amino)-2-(pyridin-4-yl)-5,6-dihydro-7H-pyrrolo[3,4-d]pyrimidin-7-one

### Compound B

In embodiments, the compound of Formula (I) is Compound C:

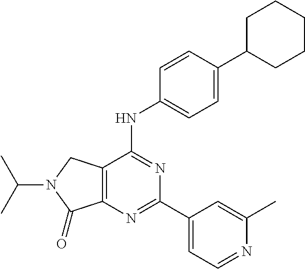 [SMILES: Cc1cc(-c2nc(Nc3ccc(C4CCCCC4)cc3)c3c(n2)C(=O)N(C(C)C)C3)ccn1]

4-[(4-cyclohexylphenyl)amino]-2-(2-methylpyridin-4-yl)-6-(propan-2-yl)-5,6-dihydro-7H-pyrrolo[3,4-d]pyrimidin-7-one

### Compound C

In embodiments, the compound of Formula (I) is Compound D:

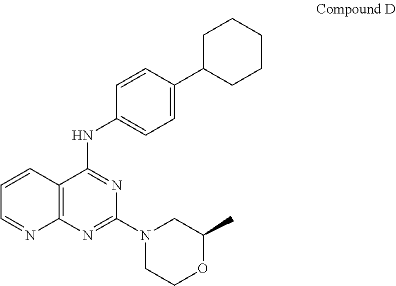 [SMILES: C[C@@H]1CN(c2nc(Nc3ccc(C4CCCCC4)cc3)c3cccnc3n2)CCO1]

N-(4-cyclohexylphenyl)-2-[(2R)-2-methylmorpholin-4-yl]pyrido[2,3-d]pyrimidin-4-amine

### Compound D

In embodiments, the compound of Formula (I) is Compound E:

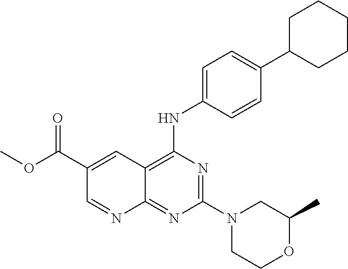 [SMILES: COC(=O)c1cnc2nc(N3CCO[C@H](C)C3)nc(Nc3ccc(C4CCCCC4)cc3)c2c1]

methyl (R)-4-((4-cyclohexylphenyl)amino)-2-(2-methylmorpholino)pyrido[2,3-d]pyrimidine-6-carboxylate

### Compound E

In embodiments, the compound of Formula (I) is Compound F:

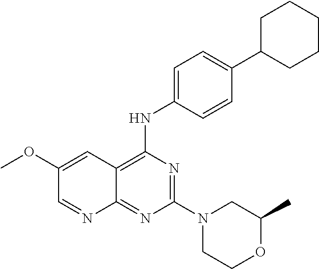 [SMILES: COc1cnc2nc(N3CCO[C@H](C)C3)nc(Nc3ccc(C4CCCCC4)cc3)c2c1]

(R)-N-(4-cyclohexylphenyl)-6-methoxy-2-(2-methylmorpholino)pyrido[2,3-d]pyrimidin-4-amine

### Compound F

In embodiments, the compound of Formula (I) is Compound G:

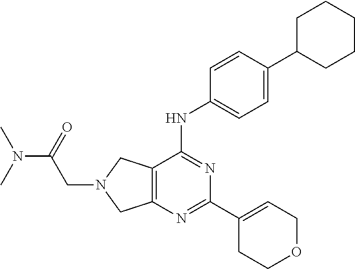 [SMILES: CN(C)C(=O)CN1Cc2nc(C3=CCOCC3)nc(Nc3ccc(C4CCCCC4)cc3)c2C1]

2-(4-((4-cyclohexylphenyl)amino)-2-(3,6-dihydro-2H-pyran-4-yl)-5,7-dihydro-6H-pyrrolo[3,4-d]pyrimidin-6-yl)-N,N-dimethylacetamide

### Compound G

In embodiments, the compound according to Formula (I) is:

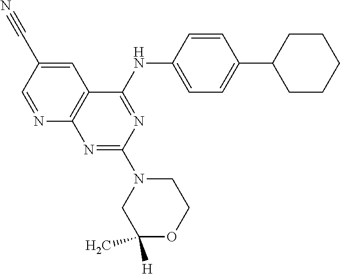 [SMILES: C[C@@H]1CN(c2nc(Nc3ccc(C4CCCCC4)cc3)c3cc(C#N)cnc3n2)CCO1]

known as (R)-4-((4-cyclohexylphenyl)amino)-2-(2-methylmorpholino)pyrido[2,3-d]pyrimidine-6-carbonitrile (also referred to as 4-[(4-cyclohexylphenyl)amino]-2-[(2R)-2-methylmorpholin-4-yl]pyrido[2,3-d]pyrimidine-6-carbonitrile or Compound H), or a pharmaceutically acceptable salt thereof.

Atoms of the compounds and salts described herein may exist as their isotopes. All compounds of Formula (I) where an atom is replaced by one or more of its isotopes (for example a compound of Formula (I) where one or more carbon atom is an ¹¹C or ¹³C carbon isotope, or where one or more hydrogen atoms is a ²H or ³H isotope, or where one or more nitrogen atoms is a ¹⁵N isotope or where one of more oxygen atoms is an ¹⁷O or ¹⁸O isotope) are encompassed herein.

Compounds herein may exist in one or more geometrical, optical, enantiomeric, and diastereomeric forms, including, but not limited to, cis- and trans-forms, E- and Z-forms, and R-, S- and meso-forms. Unless otherwise stated a reference to a particular compound includes all such isomeric forms, including racemic and other mixtures thereof. Where appropriate such isomers can be separated from their mixtures by the application or adaptation of known methods (e.g., chromatographic techniques and recrystallisation techniques). Where appropriate such isomers can be prepared by the application or adaptation of known methods. In embodiments, a single stereoisomer is obtained by isolating it from a mixture of isomers (e.g., a racemate) using, for example, chiral chromatographic separation. In embodiments, a single stereoisomer is obtained through direct synthesis from, for example, a chiral starting material.

In embodiments, there is provided a compound of Formula (I), or a pharmaceutically acceptable salt thereof, which is a single optical isomer being in an enantiomeric excess (% e.e.) of ≥95%, ≥98% or ≥99%. In embodiments, the single optical isomer is present in an enantiomeric excess (% e.e.) of ≥99%.

In embodiments, there is provided an N-oxide of a compound of Formula (I) as herein defined, or a pharmaceutically acceptable salt thereof.

Compounds of Formula (I), where R⁷ is —NR¹⁰R¹¹ (i.e., R⁷ is linked by an aliphatic N atom), may for example be prepared as described in WO2021/180952 by the reaction of a compound of Formula (V):

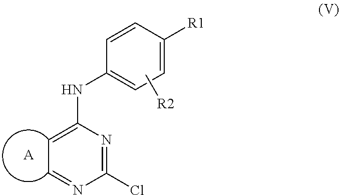

or a salt thereof, where R¹, R² and A are as defined herein, with an amine. The reaction is conveniently performed in a suitable solvent and at a suitable temperature, for example, di-isopropylethylamine in dimethylsulfoxide at a temperature of 20-100° C., or TsOH in butanol at 80° C.

When R⁷ is attached via a carbon atom, the compound of Formula (I) can be made by the reaction of a compound of Formula (V) with a boronic acid or ester of the Formula (VI), where R⁷ is as defined herein and each R is the same or different and represents —H, an aliphatic chain, or where together the two R groups form a ring with the boron and two oxygen atoms. The reaction is conveniently performed with a suitable base in the presence of a palladium catalyst and a solvent at a suitable temperature. For example, cesium carbonate or sodium carbonate and a palladium catalyst such as Pd(PPh₃)₄, in aqueous dioxane at a temperature in the range of 80-100° C.

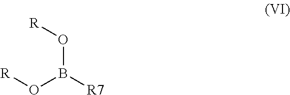

When R⁷ is linked via an aromatic N atom, the compound of Formula (I) can be made by reaction of a compound of Formula (V) with the anion of R⁷. For example, by reaction of the anion of imidazole, generated by treatment with a suitable base (for example, sodium hydride), in a suitable solvent (for example dimethylformamide), with a compound of Formula (V).

A compound of the Formula (V) may be prepared from a compound of Formula (VII), or a salt thereof, where A is as defined herein, and a compound of Formula (VIII), or a salt thereof, where R¹ and R² are as defined herein, in the presence of a base in a suitable solvent (for example, di-isopropylethylamine in tert butanol or dimethylsulfoxide) and at a suitable temperature (for example 20-100° C.).

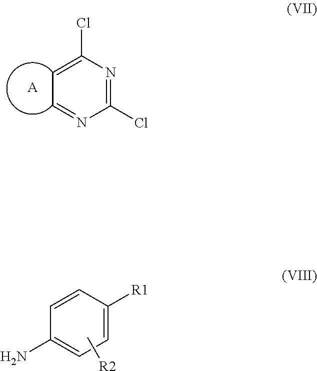

A compound of Formula (I) may also be made in one pot from the reaction between a compound of Formula (VII) with the stepwise addition of a compound of Formula (VIII) and an amine R⁷. The reaction is conveniently performed in the presence of a base (for example, di-isopropylethylamine) in a suitable solvent (dimethylsulfoxide) at a suitable temperature (for example, a temperature of 20-100° C.).

A compound of Formula (VII) may be made, for example, from a compound of Formula (IX). Suitable conditions for this transformation are heating at a temperature of about 80° C. in POCl₃ in the presence of an amine base such as diethylphenylamine.

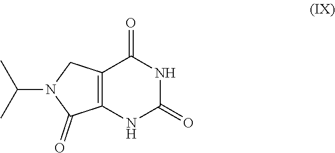

A compound of the Formula (IX) may, for example, be prepared from a compound of the Formula (X) by reaction with propan-2-amine and formaldehyde in a suitable solvent (for example, ethanol) at a suitable temperature (for example, a temperature of 0-80° C.).

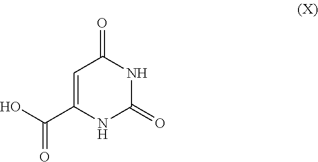 [SMILES: O=C(O)c1cc(=O)[nH]c(=O)[nH]1]

A compound of the Formula (I), when R^{5a} and R^{5b} are both H, may also be made from reaction of a compound of the Formula (XI), or a salt thereof, where R¹, R² and R⁷ are as defined herein, with a suitable amine, for example N,N-dimethylpropane-1,3-diamine. Suitable conditions for this reaction are HCl in ethanol at a temperature of about 190° C. in a sealed tube.

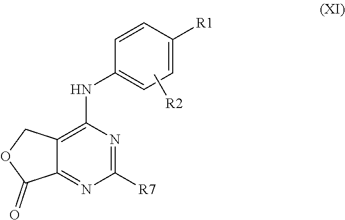

It will be appreciated that certain of the various ring substituents in the compounds herein may be introduced by standard aromatic substitution reactions or generated by conventional functional group modifications either prior to or immediately following the processes mentioned above. For example, compounds of Formula (I) may be converted into further compounds of Formula (I) by conventional functional group modifications. Such reactions and modifications include, for example, introduction of a substituent by means of an aromatic substitution reaction, C—H activation reaction, reduction of substituents, alkylation of substituents and oxidation of substituents. The reagents and reaction conditions for such procedures are well known in the chemical art. Particular examples of aromatic substitution reactions include the introduction of a halogen group.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

As a result of their KCC2 activation activity, as discussed above, the compounds of Formula (I), or pharmaceutically acceptable salts thereof are useful in therapy, for example, in the treatment of Huntington's disease mediated at least in part by KCC2. In embodiments, the compounds of Formula (I), or pharmaceutically acceptable salts thereof, increase KCC2 activity, diminish neuronal hyperexcitabilty, and provide a GABAergic effect, resulting in beneficial therapeutic effects on Huntington's disease.

Compounds of Formula (I), or pharmaceutically acceptable salts thereof are administered in combination with pridopidine, or a pharmaceutically acceptable salt thereof, for treatment of Huntington's disease in a subject diagnosed with Huntington's disease. Compounds of Formula (I), or pharmaceutically acceptable salts thereof and pridopidine, or a pharmaceutically acceptable salt thereof, may be administered in the form of a pharmaceutical composition containing both a compound of Formula (I), or a pharmaceutically acceptable salt thereof and pridopidine, or a pharmaceutically acceptable salt thereof, or as two separate pharmaceutical compositions—one containing a compound of Formula (I), or a pharmaceutically acceptable salt thereof, and one containing pridopidine, or a pharmaceutically acceptable salt thereof. In the case of two separate pharmaceutical compositions, they may be given concurrently or in spaced apart intervals such as, e.g., 5 minutes apart, 10 minutes apart, 20 minutes apart, 30 minutes apart, 40 minutes apart, 45 minutes apart, 60 minutes apart, 2 hours apart, 3 hours apart, 4 hours apart, 5 hours apart, 6 hours apart, 7 hours apart, 8 hours apart, 9 hours apart, 10 hours apart, 11 hours apart, 12 hours apart, 15 hours apart, 20 hours apart, 24 hours apart, etc.

Both organic and inorganic acids can be employed to form pharmaceutically acceptable acid addition salts of pridopidine. Illustrative acids are sulfuric, nitric, phosphoric, hydrochloric, hydrobromic, citric, acetic, lactic, tartaric, palmoic, ethane disulfonic, sulfamic, succinic, cyclohexylsulfamic, fumaric, maleic, and benzoic acid. These salts are readily prepared by methods known in the art.

The term “therapy” is intended to have its normal meaning of dealing with a disease in order to entirely or partially relieve one, some or all of its symptoms, or to correct or compensate for the underlying pathology. In embodiments, the term “therapy” may also include “prophylaxis” if “prophylaxis” is specifically referred to. The term “prophylaxis” is intended to have its normal meaning and includes primary prophylaxis to prevent the development of the disease and secondary prophylaxis whereby the disease or disorder has already developed and the patient is temporarily or permanently protected against exacerbation or worsening of the disease or the development of new symptoms associated with the disease. Nonetheless, prophylactic (preventive) and therapeutic treatment are two separate embodiments of the disclosure herein. In embodiments, the term “treatment” can be used synonymously with “therapy”. Similarly, the term “treat” can be regarded as “applying therapy” where “therapy” is as defined herein. As used herein, the terms “treat”, “treatment” or “treating” as applied to the Huntington's disease encompass any manner in which the symptoms or pathology of a condition, disorder or disease associated with Huntington's disease are ameliorated or otherwise beneficially altered. In embodiments, “treat”, “treatment” or “treating” can refer to inhibiting a disease, disorder or condition, e.g., arresting or reducing its development or at least one clinical or subclinical symptom thereof. In embodiments, “treat”, “treatment” or “treating” can refer to relieving the disease, disorder or condition, e.g., causing regression of the disease, disorder or condition or at least one of its clinical or subclinical symptoms. The terms “treat”, “treatment” or “treating” may be used interchangeably herein. The benefit to a subject being treated may be statistically significant, mathematically significant, or at least perceptible to the subject and/or the physician.

In embodiments, there is provided a compound of Formula (I), or a pharmaceutically acceptable salt thereof, and pridopidine, or a pharmaceutically acceptable salt thereof, for use in therapy for Huntington's disease. For example, in embodiments, Compound A, Compound B, Compound C, Compound D, Compound E, Compound F, Compound G, or Compound H, or a pharmaceutically acceptable salt of any of the foregoing, and pridopidine, or a pharmaceutically acceptable salt thereof, is for use in therapy for Huntington's disease. In embodiments, there is provided the use of a compound of Formula (I), or a pharmaceutically acceptable salt thereof, e.g., Compound A, Compound B, Compound C, Compound D, Compound E, Compound F, Compound G, or Compound H, or a pharmaceutically acceptable salt of any of the foregoing, and pridopidine, or a pharmaceutically acceptable salt thereof, for the manufacture of a medicament for Huntington's disease.

The term “therapeutically effective amount” refers to an amount of a compound of Formula (I) as described herein which is effective to provide “therapy” in a subject, or to “treat” a disease or disorder in a subject. In the case of Huntington's disease, the therapeutically effective amount may cause any of the changes observable or measurable in a subject as described in the definition of “therapy”, “treatment” or “prophylaxis” above. As recognized by those skilled in the art, effective amounts may vary depending on route of administration, excipient usage, and co-usage with other agents. For example, where a combination therapy is used, the amount of the compound of Formula (I) or pharmaceutically acceptable salt thereof and pridopidine, or a pharmaceutically acceptable salt thereof, as described in this specification and the amount of another pharmaceutically active agent(s) are, when combined, jointly effective to treat a Huntington's disease in the patient. In this context, the combined amounts are in a “therapeutically effective amount” if they are, when combined, sufficient to decrease the symptoms of Huntington's disease responsive to activation of KCC2 and pridopidine, or a pharmaceutically acceptable salt thereof, as described herein. Typically, such amounts may be determined by one skilled in the art by, for example, starting with the dosage range described in this specification for the compound of Formula (I) or pharmaceutically acceptable salt thereof, and pridopidine, or a pharmaceutically acceptable salt thereof, and an approved or otherwise published dosage range(s) of the other pharmaceutically active compound(s). “Subjects” or “patients” include mammals, for example, humans. The terms “subjects” and “patients” may be used interchangeably herein.

The compounds of Formula (I), or pharmaceutically acceptable salts thereof, and pridopidine, or a pharmaceutically acceptable salt thereof, may be administered in pharmaceutical compositions, including one or more pharmaceutically acceptable excipients. Therefore, in embodiments, there is provided a pharmaceutical composition including a compound of Formula (I), or a pharmaceutically acceptable salt thereof, and pridopidine, or a pharmaceutically acceptable salt thereof, and at least one pharmaceutically acceptable excipient. In embodiments, there is provided a pharmaceutical composition including a compound of Formula (I), or a pharmaceutically acceptable salt thereof, and at least one pharmaceutically acceptable excipient. In embodiments, there is provided a pharmaceutical composition including pridopidine, or a pharmaceutically acceptable salt thereof, and at least one pharmaceutically acceptable excipient.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilizers, flavorings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, films, dragees, aqueous or oily suspensions, emulsions, dispersible powders or granules, syrups or elixirs), for topical use (for example as creams, ointments, gels, lotions, transdermal patches, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution or suspension for intravenous, subcutaneous or intramuscular dosing), or as a suppository for rectal dosing. The compositions may be obtained by conventional procedures using conventional pharmaceutical excipients well known in the art. Thus, compositions intended for oral use may contain, for example, one or more coloring, sweetening, flavoring and/or preservative agents.

In embodiments, a pharmaceutical composition herein may contain a compound according to Formula (I) in any of the amounts set forth herein and a diluent in an amount from 30% to 90% by weight. In embodiments, a pharmaceutical composition herein may contain pridopidine, or a pharmaceutically acceptable salt thereof, in any of the amounts set forth herein and a diluent in an amount from 30% to 90% by weight. In embodiments, a pharmaceutical composition herein may contain a compound according to Formula (I) in any of the amounts set forth herein, and pridopidine, or a pharmaceutically acceptable salt thereof, and a diluent in an amount from 30% to 90% by weight. Examples of diluents include lactose, microcrystalline cellulose, starch, calcium phosphate, calcium carbonate, sucrose, mannitol, maltodextrin and sorbitol.

In embodiments, a pharmaceutical composition herein may contain a compound according to Formula (I) and a lubricant in an amount, e.g., from 0.25% to 5.0% by weight. In embodiments, a pharmaceutical composition herein may contain pridopidine, or a pharmaceutically acceptable salt thereof, in any of the amounts set forth herein and a lubricant in an amount, e.g., from 0.25% to 5.0% by weight. In embodiments, a pharmaceutical composition herein may contain a compound according to Formula (I) in any of the amounts set forth herein, and pridopidine, or a pharmaceutically acceptable salt thereof, and a lubricant in an amount, e.g., from 0.25% to 5.0% by weight. Examples of lubricants include magnesium stearate, stearic acid, sodium stearyl fumarate, talc, polyethylene glycols and silicon dioxide. Examples of water-soluble lubricants include sodium benzoate, polyethylene glycol, and adipic acid.

In embodiments, a pharmaceutical composition herein may contain a compound according to Formula (I) and a disintegrant in an amount, e.g., from 1.0% to 10.0% by weight. In embodiments, a pharmaceutical composition herein may contain pridopidine, or a pharmaceutically acceptable salt thereof, in any of the amounts set forth herein and a disintegrant in an amount, e.g., from 1.0% to 10.0% by weight. In embodiments, a pharmaceutical composition herein may contain a compound according to Formula (I) in any of the amounts set forth herein, and pridopidine, or a pharmaceutically acceptable salt thereof, and a disintegrant in an amount, e.g., from 1.0% to 10.0% by weight. There are two classes of disintegrants: traditional disintegrants, such as starch, and super disintegrants, which include croscarmellose sodium, crospovidone, and sodium starch glycolate.

The compound of Formula (I), or a pharmaceutically acceptable salt thereof, will normally be administered to a subject, e.g., a warm-blooded animal at a unit dose within the range 2.5-5000 mg/m² body area of the animal, or approximately 0.05-100 mg/kg, and this normally provides a therapeutically-effective dose. A unit dose form such as a tablet, capsule, film, patch, vial will can contain, for example 0.1-500 mg of the compound of Formula (I), or a pharmaceutically acceptable salt thereof. A therapeutically effective amount of pridopidine, or a pharmaceutically acceptable salt thereof, is from about 0.01 mg to about 500 mg/kg body weight daily, preferably 0.1-10 mg/kg body weight daily. A unit dose form such as a tablet, capsule, film, patch, vial will can contain, for example 0.1-500 mg of pridopidine, or a pharmaceutically acceptable salt thereof. The daily dose of either Formula (I), or a pharmaceutically acceptable salt thereof, and pridopidine, or a pharmaceutically acceptable salt thereof, will necessarily be varied depending upon the host treated, the particular route of administration, any therapies being co-administered, and the severity of the illness being treated.

The pharmaceutical compositions described herein include compounds of Formula (I), or a pharmaceutically acceptable salt thereof, and/or pridopidine, or a pharmaceutically acceptable salt thereof, for use in therapy for Huntington's disease. As such, in embodiments, there is provided a pharmaceutical composition for use in therapy for Huntington's disease, including a compound of Formula (I), or a pharmaceutically acceptable salt thereof, and/or pridopidine, or a pharmaceutically acceptable salt thereof, and at least one pharmaceutically acceptable excipient.

In embodiments, there is provided a pharmaceutical composition for use in the treatment of Huntington's disease in which activation of KCC2 and antagonism of the D2 receptor is beneficial, including a compound of Formula (I), or a pharmaceutically acceptable salt thereof, and/or pridopidine, or a pharmaceutically acceptable salt thereof, and at least one pharmaceutically acceptable excipient.

In embodiments, treatment of Huntington's disease is implemented by administering to a subject, e.g., a human, in need thereof or diagnosed with Huntington's disease, about 0.01 mg to about 1500 mg of a compound according to Formula (I), or a pharmaceutically acceptable salt thereof, such as Compound A, Compound B, Compound C, Compound D, Compound E, Compound F, Compound G, or Compound H, or a pharmaceutically acceptable salt of any of the foregoing, in combination with pridopidine, or a pharmaceutically acceptable salt thereof. In embodiments, methods include treating Huntington's disease by administering to a subject, e.g., a human, in need thereof or diagnosed with Huntington's disease about 0.01 mg to about 1500 mg of a compound according to Formula (I), or a pharmaceutically acceptable salt thereof. In embodiments, the amount of a compound according to Formula (I), or a pharmaceutically acceptable salt thereof, can be, e.g., between 0.1 and 1500 mg/day, or 0.01 mg/kg/day to 15 mg/kg/day, for treatment of Huntington's disease. For example, the daily dosage can be, e.g., in the range of about 0.01 to 1500 mg, 0.1 to 1250 mg, 0.1 to 1000 mg, 0.1 to 750 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 1 to 1500 mg, 1 to 1000 mg, 1 to 500 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 1500 mg, 5 to 1000 mg, 5 to 500 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 1500 mg, 10 to 1000 mg, 10 to 500 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 1500 mg, 15 to 1000 mg, 15 to 500 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 1500 mg, 20 to 1000 mg, 20 to 500 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 1500 mg, 25 to 1000 mg, 25 to 500 mg, 25 to 300 mg, 25 to 250 mg, 25 to 200 mg, 25 to 175 mg, 25 to 150 mg, 25 to 125 mg, 25 to 100 mg, 25 to 75 mg, 25 to 50 mg, 25 to 30 mg, 30 to 1500 mg, 30 to 1000 mg, 30 to 500 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 35 to 1500 mg, 35 to 1000 mg, 35 to 500 mg, 35 to 300 mg, 35 to 250 mg, 35 to 200 mg, 35 to 175 mg, 35 to 150 mg, 35 to 125 mg, 35 to 100 mg, 35 to 75 mg, 35 to 50 mg, 40 to 1500 mg, 40 to 1000 mg, 40 to 500 mg, 40 to 300 mg, 40 to 250 mg, 40 to 200 mg, 40 to 175 mg, 40 to 150 mg, 40 to 125 mg, 40 to 100 mg, 40 to 75 mg, 40 to 50 mg, 50 to 1500 mg, 50 to 1000 mg, 50 to 500 mg, 50 to 300 mg, 50 to 250 mg, 50 to 200 mg, 50 to 175 mg, 50 to 150 mg, 50 to 125 mg, 50 to 100 mg, 50 to 75 mg, 75 to 1500 mg, 75 to 1000 mg, 75 to 500 mg, 75 to 300 mg, 75 to 250 mg, 75 to 200 mg, 75 to 175 mg, 75 to 150 mg, 75 to 125 mg, 75 to 100 mg, 100 to 1500 mg, 100 to 1000 mg, 100 to 500 mg, 100 to 300 mg, 100 to 250 mg, 100 to 200 mg, 100 to 175 mg, 100 to 150 mg, 100 to 125 mg, 125 to 1500 mg, 125 to 1000 mg, 125 to 500 mg, 125 to 300 mg, 125 to 250 mg, 125 to 200 mg, 125 to 175 mg, 125 to 150 mg, 150 to 1500 mg, 150 to 1000 mg, 150 to 500 mg, 150 to 300 mg, 150 to 250 mg, 150 to 200 mg, 150 to 175 mg, 175 to 1500 mg, 175 to 1000 mg, 175 to 500 mg, 175 to 300 mg, 175 to 250 mg, 175 to 200 mg, 200 to 1500 mg, 200 to 1000 mg, 200 to 500 mg, 200 to 300 mg, 200 to 250 mg, 250 to 1500 mg, 250 to 1000 mg, 250 to 500 mg, 250 to 300 mg, 7.5 to 15 mg, 2.5 to 5 mg, 1 to 5 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 7.5 mg, 10 mg, 12.5 mg, 15 mg, 17.5 mg, 20 mg, 22.5 mg, 25 mg, 27.5 mg, 30 mg, 35 mg, 40 mg, 45 mg, 50 mg, 75 mg, 100 mg, 125 mg, 150 mg, 175 mg, 200 mg, 225 mg, 250 mg, 275 mg, 300 mg, 400 mg and 500 mg being examples.

In embodiments, pharmaceutical compositions for treating Huntington's disease may include a compound according to Formula (I), or a pharmaceutically acceptable salt thereof, in an amount of, e.g., about 0.01 to 1500 mg, 0.01 to 1250 mg, 0.01 to 1000 mg, 0.01 to 750 mg, 0.01 to 500 mg, 0.01 to 250 mg, 0.01 to 100 mg, 0.01 to 50 mg, 0.01 to 25 mg, 0.01 to 10 mg, 0.01 to 5 mg, 0.01 to 1 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.5 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 1 mg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.1 mg, 0.25 mg, 0.5 mg, 0.75 mg, 1 mg, 2.5 mg, 5 mg, 7.5 mg, 10 mg, 12.5 mg, 15 mg, 17.5 mg, 20 mg, 22.5 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples.

Typically, dosages of Formula (I), or a pharmaceutically acceptable salt thereof, may be administered to a subject diagnosed with Huntington's disease once, twice, three or four times daily, every other day, once weekly, or once a month. In embodiments, a compound according to Formula (I), or a pharmaceutically acceptable salt thereof, is administered to a subject diagnosed with Huntington's disease once a day (e.g., morning or night), or twice a day, (e.g., morning and evening), three times a day (e.g., at breakfast, lunch, and dinner, or every 8 hours), or four times a day (e.g., breakfast, lunch, dinner and at bedtime) at a dose of 0.01-1000 mg/administration. In embodiments, the pharmaceutical compositions described herein may be administered by continuous infusion.

In embodiments, a compound according to Formula (I), or a pharmaceutically acceptable salt thereof, is administered to a subject diagnosed with Huntington's disease herein in an amount of 1500 mg/per day, 1400 mg/per day, 1300 mg/per day, 1200 mg/per day, 1000 mg/per day, 900 mg/per day, 800 mg/per day, 700 mg/per day, 600 mg/per day, 500 mg/per day, 400 mg/per day, 300 mg/per day, 200 mg/per day, 100 mg/per day, 95 mg/per day, 90 mg/per day, 85 mg/per day, 80 mg/per day, 75 mg/per day, 70 mg/per day, 65 mg/per day, 60 mg/per day, 55 mg/per day, 50 mg/per day, 45 mg/per day, 40 mg/per day, 35 mg/per day, 30 mg/per day, 25 mg/per day, 20 mg/per day, 15 mg/per day, 10 mg/per day, 5 mg/per day, 4 mg/per day, 3 mg/per day, 3 mg/per day, 2 mg/per day, 1 mg/per day, in one or more doses. Dosages can be lower for infants and children than for adults. In embodiments, an infant or pediatric dose can be about 0.1 to 1500 mg per day once or in 2, 3 or 4 divided doses. In embodiments, a pediatric dose can be 0.05 mg/kg/day to 1500 mg/kg/day. In embodiments, the subject may be started at a low dose and the dosage is escalated over time. It should be understood that the above amounts are exemplary and doses of a compound according to Formula (I), or a pharmaceutically acceptable salt thereof, can include different amounts and varying ranges within a continuum between the minimum or maximum amounts described above in connection with a compound according to Formula (I), or a pharmaceutically acceptable salt thereof.

In embodiments, pharmaceutical compositions for treating Huntington's disease may include about 0.1 mg to about 1000 mg, about 22.5 mg to about 315 mg or about 90 mg to about 315 mg of pridopidine or a pharmaceutical salt thereof. In embodiments, the amount of pridopidine or a pharmaceutical salt thereof in a pharmaceutical composition is about 22.5 mg, about 45 mg, about 67.5, mg, about 90 mg, about 100 mg, about 112.5 mg, about 125 mg, about 135 mg, about 150 mg, about 180 mg, about 200 mg, about 250 mg, or about 315 mg. In embodiments, the amount of pridopidine or a pharmaceutical salt thereof in a pharmaceutical composition is about 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 1 to 450 mg, 1 to 400 mg, 1 to 350 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 80 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, or 40 to 50 mg.

In embodiments, the amount of pridopidine or a pharmaceutical salt thereof administered is 10 mg/day to 350 mg/day, 22.5 mg/day to 315 mg/day or 90 mg/day to 315 mg/day. In embodiments, the amount of pridopidine or a pharmaceutical salt thereof administered is about 22.5 mg/day, about 45 mg/day, about 67.5 mg/day, about 90 mg/day, about 100 mg/day, about 112.5 mg/day, about 125 mg/day, about 135 mg/day, about 150 mg/day, about 180 mg/day, about 200 mg/day, about 225 mg/day, about 250 mg/day, or about 315 mg/day.

In embodiments, methods include treating Huntington's disease by administering to a subject, e.g., a human, in need thereof or diagnosed with Huntington's disease about 0.01 mg to about 1000 mg of pridopidine or a pharmaceutical salt thereof. In embodiments, the amount of pridopidine, or a pharmaceutically acceptable salt thereof, can be, e.g., between 0.1 and 1000 mg/day, or 0.01 mg/kg/day to 15 mg/kg/day, for treatment of Huntington's disease. For example, the daily dosage can be, e.g., in the range of about 0.01 to 1500 mg, 0.1 to 1250 mg, 0.1 to 1000 mg, 0.1 to 750 mg, 0.1 to 500, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, or 0.1 to 1 mg in one or more doses. Dosages can be lower for infants and children than for adults. In embodiments, an infant or pediatric dose can be about 0.01 to 500 mg per day once or in 2, 3 or 4 divided doses. In embodiments, a pediatric dose can be 0.01 mg/kg/day to 100 mg/kg/day. In embodiments, the subject may be started at a low dose and the dosage is escalated over time. It should be understood that the above amounts are exemplary and doses of pridopidine, or a pharmaceutically acceptable salt thereof, can include different amounts and varying ranges within a continuum between the minimum or maximum amounts described above.

Typically, dosages of pridopidine, or a pharmaceutical salt thereof, may be administered to a subject diagnosed with Huntington's disease once, twice, three or four times daily, every other day, once weekly, or once a month. For example, pridopidine, or a pharmaceutical salt thereof, may be administered to a subject diagnosed with Huntington's disease at 45 mg twice a day, 67.5 mg twice a day, 90 mg twice a day or 112.5 mg twice a day. In embodiments, pridopidine, or a pharmaceutically acceptable salt thereof, is administered to a subject diagnosed with Huntington's disease once a day (e.g., morning or night), or twice a day, (e.g., morning and evening), three times a day (e.g., at breakfast, lunch, and dinner, or every 8 hours), or four times a day (e.g., breakfast, lunch, dinner and at bedtime) at a dose of 0.01-500 mg/administration. In embodiments, the pharmaceutical compositions described herein may be administered by continuous infusion.

In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 1 hour after administration to the subject. In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 2 hours after administration to the subject. In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 3 hours after administration to the subject. In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 4 hours after administration to the subject. In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 6 hours after administration to the subject. In embodiments, methods of treating Huntington's disease are provided which include administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, wherein the composition provides improvement in one or more symptoms of Huntington's disease for more than 8, 10, 12, 14, 16, 18, 20, 22 or 24 hours after administration to the subject. In embodiments, improvement in at least one or two or more symptoms for 12 hours after administration of the pharmaceutical composition to the subject is provided in accordance with the present disclosure. In embodiments, the pharmaceutical compositions provide improvement in next day functioning of the subject. For example, the pharmaceutical compositions may provide improvement in one or more symptoms of Huntington's disease for more than about, e.g., 2 hours, 4 hours, 6 hours, 8 hours, 10 hours, 12 hours, 14 hours, 16 hours, 18 hours, 20 hours, 22 hours or 24 hours after administration and waking from a night of sleep.

In embodiments, provided herein are methods of treating Huntington's disease including administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, in an effective amount, after an early sign of one or more symptoms of Huntington's disease is detected to reduce or prevent further symptoms. In embodiments, a continuing regimen of administration of active agents herein according to a compound of Formula (I), or a pharmaceutically acceptable salt thereof, and/or pridopidine, or a pharmaceutical salt thereof, is effective to reduce or prevent occurrence of symptoms associated with Huntington's disease.

In embodiments, the methods and compositions described herein are effective to reduce, delay, or prevent one or more other clinical symptoms of Huntington's disease. For example, the effect of a composition including a compound according to Formula (I), or a pharmaceutically acceptable salt thereof, and/or pridopidine, or a pharmaceutical salt thereof, on a particular symptom, pharmacologic, or physiologic indicator can be compared to an untreated subject, or the condition of the subject prior to treatment. In embodiments, the symptom, pharmacologic, and/or physiologic indicator is measured in a subject prior to treatment, and again one or more times after treatment is initiated. In embodiments, the control is a reference level, or average determined based on measuring the symptom, pharmacologic, or physiologic indicator in one or more subjects that do not have the disease, disorder or condition to be treated (e.g., healthy subjects). In embodiments, the effect of the treatment is compared to a conventional treatment that is known in the art.

Indeed, clinical efficacy of treatment can be monitored using any method known in the art. Measurable parameters to monitor efficacy will depend on the condition being treated. For monitoring the status or improvement of symptoms of Huntington's disease various scales and tools are known. For example, the Positive and Negative Syndrome Scale, Brief Psychiatric Rating Scale, Scale for the Assessment of Positive Symptoms, Scale for the Assessment of Negative Symptoms, and the Scale of Prodromal Symptoms.

Effective treatment of Huntington's disease may be established by showing reduction in the frequency or severity of symptoms (e.g., more than, e.g., 10%, 20%, 30% 40%, 50% or more) after a period of time compared with baseline. For example, after a baseline period of 1 month, the subjects may be randomly allocated a compound according to Formula (I), or a pharmaceutically acceptable salt thereof, and/or pridopidine, or a pharmaceutical salt thereof, or placebo as add-on therapy to standard therapies, during a double-blind period of, e.g., 2 months.

In embodiments, primary outcome measurements may include the percentage of responders on a compound according to Formula (I), or a pharmaceutically acceptable salt thereof, and/or pridopidine, or a pharmaceutical salt thereof, and on placebo, defined as having experienced at least a 10% to 50% or more reduction of symptoms during the second month of the double-blind period compared with baseline.

Cognitive impairment (aka impairment of cognition) may be associated with subjects diagnosed with Huntington's disease. Cognitive impairment may be measured against normal cognitive function, which refers to the normal physiologic activity of the brain, including, but not limited to, one or more of the following: mental stability, memory/recall abilities, problem solving abilities, reasoning abilities, thinking abilities, judging abilities, ability to discriminate or make choices, capacity for learning, ease of learning, perception, intuition, attention, and awareness, as measured by any criteria suitable in the art.

Cognitive impairment also includes deficits in mental activities that are mild or that otherwise do not significantly interfere with daily life. Mild cognitive impairment (MCI) is an example of such a condition. A subject with mild cognitive impairment may display symptoms of dementia or delusions (e.g., difficulties with language or memory).

One skilled in the art will appreciate that there are numerous human and animal models that may be used to evaluate and compare the relative safety and efficacy of compounds described herein for the treatment of cognitive impairment associated with a Huntington's disease. In humans, cognitive function may be measured, for example and without limitation, by the clinical global impression of change scale (CGI); the Mini Mental State Exam (MMSE) (aka the Folstein Test); the Neuropsychiatric Inventory (NPI); the Clinical Dementia Rating Scale (CDR); the Cambridge Neuropsychological Test Automated Battery (CANTAB), the Sandoz Clinical Assessment-Geriatric (SCAG) scale, the Benton Visual Retention Test (BVRT), Montreal Cognitive Assessment (MoCA) or Digit Symbol Substitution Test (DSST).

In animal model systems, cognitive function may be measured in various conventional ways known in the art, including using a Morris Water Navigation Task, Barnes maze, radial arm maze task, T maze and the like. Other tests known in art may also be used to assess cognitive function, such as novel object recognition and odor recognition tasks.

Cognitive function may also be measured using imaging techniques such as Positron Emission Tomography (PET), functional magnetic resonance imaging (fMRI), Single Photon Emission Computed Tomography (SPECT), or any other imaging technique that allows one to measure brain function. In animals, cognitive function may also be measured with electrophysiological techniques.

In embodiments, a compound according to Formula (I), or a pharmaceutically acceptable salt thereof, and/or pridopidine, or a pharmaceutical salt thereof, is administered via a pharmaceutical composition for treatment of Huntington's disease. Pharmaceutical compositions herein encompass dosage forms. Dosage forms herein encompass unit doses. In embodiments, as discussed below, various dosage forms including conventional formulations and modified release formulations can be administered one or more times daily. Any suitable route of administration may be utilized, e.g., enteral or parenteral including oral, rectal, nasal, pulmonary, vaginal, sublingual, transdermal, intravenous, intraarterial, intramuscular, intraperitoneal and subcutaneous routes. As mentioned previously, suitable dosage forms include tablets, capsules, oral liquids, powders, aerosols, transdermal modalities such as topical liquids, patches, creams and ointments, parenteral formulations and suppositories.

In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile of the compound according to Formula (I), or a pharmaceutically acceptable salt thereof, and/or pridopidine, or a pharmaceutical salt thereof, wherein the in vivo plasma profile of the compound according to Formula (I), or a pharmaceutically acceptable salt thereof, and/or pridopidine, or a pharmaceutical salt thereof, in the subject 10 hours after administration of the compound according to Formula (I), or a pharmaceutically acceptable salt thereof, and/or pridopidine, or a pharmaceutical salt thereof, is reduced by more than 50% and the method provides improvement in the subject for more than 10, 12, 14, 16, 18, 20, 22 or 24 hours after administration.

In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 55% and the method provides improvement in the subject for more than 10, 12, 14, 16, 18, 20, 22 or 24 hours after administration. In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 55% and the method provides improvement in the subject for more than 10, 12, 14, 16, 18, 20, 22 or 24 hours after administration.

In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 60% and the method provides improvement in the subject for more than 10, 12, 14, 16, 18, 20, 22 or 24 hours after administration. In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 60% and the method provides improvement in the subject for more than 10, 12, 14, 16, 18, 20, 22 or 24 hours after administration in the subject for more than 10, 12, 14, 16, 18, 20, 22 or 24 hours after administration.

In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 65% and the method provides improvement in the subject for more than 10, 12, 14, 16, 18, 20, 22 or 24 hours after administration. In embodiments, provided herein are methods of treating Huntington's disease including administering to a subject in need thereof or diagnosed with Huntington's disease a compound according to Formula (I), or a pharmaceutically acceptable salt thereof, and/or pridopidine, or a pharmaceutical salt thereof, which provides an in vivo plasma profile, wherein the in vivo plasma profile of a compound according to Formula (I), or a pharmaceutically acceptable salt thereof, and/or pridopidine, or a pharmaceutical salt thereof, in the subject 10 hours after administration of a compound according to Formula (I), or a pharmaceutically acceptable salt thereof, and/or pridopidine, or a pharmaceutical salt thereof, is reduced by more than 65% and the method provides improvement in the subject for more than 10, 12, 14, 16, 18, 20, 22 or 24 hours after administration.

In embodiments, provided herein are methods and pharmaceutical compositions for treating Huntington's disease wherein the amount of a compound according to Formula (I), or a pharmaceutically acceptable salt thereof, and/or pridopidine, or a pharmaceutical salt thereof, within the subject about 4 hours after administration of the pharmaceutical composition is less than about 75% of the administered dose. In embodiments, provided herein are methods wherein the amount of a compound according to Formula (I), or a pharmaceutically acceptable salt thereof, and/or pridopidine, or a pharmaceutical salt thereof, within the subject about, e.g., 6 hours, 8 hours, 10 hours, 12 hours, 15 hours, or 20 hours after administration of the pharmaceutical composition is less than about 75% of the administered dose.

In embodiments, provided herein are methods and pharmaceutical compositions for treating Huntington's disease wherein the amount of a compound according to Formula (I), or a pharmaceutically acceptable salt thereof, and/or pridopidine, or a pharmaceutical salt thereof, within the subject about 4 hours after administration of the pharmaceutical composition is less than about 80% of the administered dose. In embodiments, provided herein are methods and pharmaceutical compositions wherein the amount of a compound according to Formula (I), or a pharmaceutically acceptable salt thereof, and/or pridopidine, or a pharmaceutical salt thereof, within the subject about, e.g., 6 hours, 8 hours, 10 hours, 12 hours, 15 hours, or 20 hours after administration of the pharmaceutical composition is less than about 80% of the administered dose.

In embodiments, provided herein are methods and pharmaceutical compositions for treating Huntington's disease wherein the amount of a compound according to Formula (I), or a pharmaceutically acceptable salt thereof, and/or pridopidine, or a pharmaceutical salt thereof, within the subject about 4 hours after administration of the pharmaceutical composition is between about 65% to about 85% of the administered dose. In embodiments, the amount of a compound according to Formula (I), or a pharmaceutically acceptable salt thereof, and/or pridopidine, or a pharmaceutical salt thereof, within the subject after about, e.g., 6 hours, 8 hours, 10 hours, 12 hours, 15 hours, or 20 hours after administration of the pharmaceutical composition is between about 65% to about 85% of the administered dose.

In embodiments, as mentioned previously, pharmaceutical compositions herein may be provided with conventional release or modified release profiles. Pharmaceutical compositions may be prepared using a pharmaceutically acceptable “carrier” or “excipient” composed of materials that are considered safe and effective. The “carrier” includes all components present in the pharmaceutical formulation other than the active ingredient or ingredients. The term “carrier” includes, but is not limited to, diluents, binders, lubricants, disintegrants, fillers, and coating compositions. Those with skill in the art are familiar with such pharmaceutical carriers and methods of compounding pharmaceutical compositions using such carriers.

In embodiments, pharmaceutical compositions herein are modified release dosage forms which provide modified release profiles. Modified release profiles may exhibit immediate release, delayed release, or extended release profiles. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. Super disintegrants may form a majority of the dosage form. This mode of administration can be beneficial to people who may have problems swallowing tablets whether it be from physical infirmity or psychiatric in nature. Subjects with Huntington's disease may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which a compound according to Formula (I), or a pharmaceutically acceptable salt thereof, and/or pridopidine, or a pharmaceutical salt thereof, is applied to beads, e.g., confectioners nonpareil beads, and then coated with conventional release retarding materials such as waxes, enteric coatings and the like. In embodiments, beads can be formed in which a compound according to Formula (I), or a pharmaceutically acceptable salt thereof, and/or pridopidine, or a pharmaceutical salt thereof, is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as a compound according to Formula (I), or a pharmaceutically acceptable salt thereof, and/or pridopidine, or a pharmaceutical salt thereof, at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles is covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles is covered with a coating which delays release of the drug.

Delayed release dosage forms are known to those skilled in the art. For example, coated delayed release beads or granules in which a compound according to Formula (I), or a pharmaceutically acceptable salt thereof, and/or pridopidine, or a pharmaceutical salt thereof, is applied to beads, e.g., confectioners nonpareil beads, and then coated with conventional release delaying materials such as waxes, enteric coatings and the like. In embodiments, beads can be formed in which a compound according to Formula (I), or a pharmaceutically acceptable salt thereof, and/or pridopidine, or a pharmaceutical salt thereof, is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of a compound according to Formula (I), or a pharmaceutically acceptable salt thereof, and/or pridopidine, or a pharmaceutical salt thereof, can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, a compound according to Formula (I), or a pharmaceutically acceptable salt thereof, and/or pridopidine, or a pharmaceutical salt thereof, is incorporated into porous inert carriers that provide delayed release profiles. In embodiments, the porous inert carriers incorporate channels or passages from which the drug diffuses into surrounding fluids. In embodiments, a compound according to Formula (I), or a pharmaceutically acceptable salt thereof, and/or pridopidine, or a pharmaceutical salt thereof, is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion or implantation into the body and may be packaged in either single-dose or multi-dose containers. In embodiments, liquid pharmaceutical compositions for parenteral administration to a subject include a compound according to Formula (I), or a pharmaceutically acceptable salt thereof, and/or pridopidine, or a pharmaceutical salt thereof, in any of the respective amounts described above. In embodiments, the pharmaceutical compositions for parenteral administration are formulated as a total volume of about, e.g., 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, pharmaceutical compositions for parenteral administration include respective amounts described above for a compound according to Formula (I), or a pharmaceutically acceptable salt thereof and/or pridopidine, or a pharmaceutical salt thereof. In embodiments, pharmaceutical compositions for parenteral administration include about 0.05 mg to about 500 mg a compound according to Formula (I), or a pharmaceutically acceptable salt thereof and/or pridopidine, or a pharmaceutical salt thereof. In embodiments, pharmaceutical compositions for parenteral administration to a subject include a compound according to Formula (I), or a pharmaceutically acceptable salt thereof, and/or pridopidine, or a pharmaceutical salt thereof, at a respective concentration of about 0.005 mg/ml to about 500 mg/ml. In embodiments, the pharmaceutical composition for parenteral administration includes a compound according to Formula (I), or a pharmaceutically acceptable salt thereof, and/or pridopidine, or a pharmaceutical salt thereof, at a respective concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes a compound according to Formula (I), or a pharmaceutically acceptable salt thereof, and/or pridopidine, or a pharmaceutical salt thereof, at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for parenteral administration is provided wherein the pharmaceutical composition is stable for at least six months. In embodiments, the pharmaceutical compositions for parenteral administration exhibit no more than about 5% decrease in a compound according to Formula (I), or a pharmaceutically acceptable salt thereof, and/or pridopidine, or a pharmaceutical salt thereof, e.g., 3 months or 6 months. In embodiments, the amount of a compound according to a compound according to Formula (I), or a pharmaceutically acceptable salt thereof, and/or pridopidine, or a pharmaceutical salt thereof, degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a subject in need thereof.

The pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of a compound according to Formula (I), or a pharmaceutically acceptable salt thereof, and/or pridopidine, or a pharmaceutical salt thereof, used in the composition. Thus, parenteral compositions are provided wherein there is no incompatibility between any of the components of the dosage form.

In embodiments, parenteral compositions including a compound according to Formula (I), or a pharmaceutically acceptable salt thereof, and/or pridopidine, or a pharmaceutical salt thereof, include a stabilizing amount of at least one excipient. For example, excipients may be selected from the group consisting of buffering agents, solubilizing agents, tonicity agents, antioxidants, chelating agents, antimicrobial agents, and preservatives. One skilled in the art will appreciate that an excipient may have more than one function and be classified in one or more defined group.

In embodiments, parenteral compositions including a compound according to Formula (I), or a pharmaceutically acceptable salt thereof, and/or pridopidine, or a pharmaceutical salt thereof, and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions may be administered as needed, e.g., once, twice, thrice or four or more times daily, or continuously depending on the subject's needs.

In embodiments, parenteral compositions of a compound according to Formula (I), or a pharmaceutically acceptable salt thereof, and/or pridopidine, or a pharmaceutical salt thereof, are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

In embodiments, provided herein are methods of treating Huntington's disease including administering to a subject in need thereof or diagnosed with Huntington's disease a pharmaceutical composition including a compound according to Formula (I), or a pharmaceutically acceptable salt thereof, and/or pridopidine, or a pharmaceutical salt thereof, in a respective amount described herein, wherein the composition provides an in vivo plasma profile of a compound according to Formula (I), or a pharmaceutically acceptable salt thereof, and/or pridopidine, or a pharmaceutical salt thereof, with a Cₘₐₓ less than about 800 ng/ml. In embodiments, the composition provides improvement for more than 6 hours after administration to the subject.

In embodiments, pharmaceutical compositions including a compound according to Formula (I), or a pharmaceutically acceptable salt thereof, and/or pridopidine, or a pharmaceutical salt thereof, provide an in vivo plasma profile of a compound according to Formula (I), or a pharmaceutically acceptable salt thereof, and/or pridopidine, or a pharmaceutical salt thereof with a Cₘₐₓ of less than about, e.g., 2000 ng/ml, 1000 ng/ml, 850 ng/ml, 800 ng/ml, 750 ng/ml, 700 ng/ml, 650 ng/ml, 600 ng/ml, 550 ng/ml, 450 ng/ml, 400 ng/ml 350 ng/ml, or 300 ng/ml and wherein the composition provides improvement of next day functioning of the subject. In embodiments, the pharmaceutical composition provides an in vivo plasma profile of a compound according to Formula (I), or a pharmaceutically acceptable salt thereof, and/or pridopidine, or a pharmaceutical salt thereof, with a Cₘₐₓ less than about, e.g., 250 ng/ml, 200 ng/ml 150 ng/ml, or 100 ng/ml and wherein the composition provides improvement of next day functioning of the subject. In embodiments, the pharmaceutical composition provides improvement in one or more symptoms of Huntington's disease for more than 6 hours after administration.

In embodiments, provided herein are methods of treating Huntington's disease including administering to a subject in need thereof or diagnosed with Huntington's disease a pharmaceutical composition containing a compound according to Formula (I), or a pharmaceutically acceptable salt thereof, and/or pridopidine, or a pharmaceutical salt thereof, wherein the composition provides a consistent in vivo plasma profile of a compound according to Formula (I), or a pharmaceutically acceptable salt thereof, and/or pridopidine, or a pharmaceutical salt thereof with a AUC_{0-∞} of less than about 900 ng·hr/ml. In embodiments, the pharmaceutical composition provides improvement in next day functioning of the subject. In embodiments, the compositions provide an in vivo plasma profile of a compound according to Formula (I), or a pharmaceutically acceptable salt thereof, and/or pridopidine, or a pharmaceutical salt thereof, with a AUC_{0-∞} of less than about, e.g., 850 ng·hr/ml, 800 ng·hr/ml, 750 ng·hr/ml, or 700 ng·hr/ml and wherein the pharmaceutical composition provides improvement of next day functioning of the subject. In embodiments, the composition provides improvement in symptoms of Huntington's disease for more than 6 hours after administration.

In embodiments, provided herein are methods of treating Huntington's disease including administering to a subject in need thereof or diagnosed with Huntington's disease a pharmaceutical composition comprising a compound according to Formula (I), or a pharmaceutically acceptable salt thereof, and/or pridopidine, or a pharmaceutical salt thereof, wherein the pharmaceutical composition provides an in vivo plasma profile of a compound according to Formula (I), or a pharmaceutically acceptable salt thereof, and/or pridopidine, or a pharmaceutical salt thereof, with a AUC_{0-∞} of less than about, e.g., 650 ng·hr/ml, 600 ng·hr/ml, 550 ng·hr/ml, 500 ng·hr/ml, or 450 ng·hr/ml. In embodiments, the composition provides an in vivo plasma profile of a compound according to Formula (I), or a pharmaceutically acceptable salt thereof, and/or pridopidine, or a pharmaceutical salt thereof, with a AUC_{0-∞} of less than about, e.g., 400 ng·hr/ml, 350 ng·hr/ml, 300 ng·hr/ml, 250 ng·hr/ml, or 200 ng·hr/ml. In embodiments, the pharmaceutical composition provides an in vivo plasma profile of a compound according to Formula (I), or a pharmaceutically acceptable salt thereof, and/or pridopidine, or a pharmaceutical salt thereof, with a AUC_{0-∞} of less than about, e.g., 150 ng·hr/ml, 100 ng·hr/ml, 75 ng·hr/ml, or 50 ng·hr/ml. In embodiments, the pharmaceutical composition provides improvement of next day functioning of the subject after administration for more than, e.g., 4 hours, 6 hours, 8 hours, 10 hours, or 12 hours, after administration of the composition to the subject.

In embodiments, the Tₘₐₓ provided by a pharmaceutical composition containing a compound according to Formula (I), or a pharmaceutically acceptable salt thereof, and/or pridopidine, or a pharmaceutical salt thereof, is less than 3 hours. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 2.5 hours. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 2 hours. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 1.5 hours. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 1 hour. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 0.5 hour. In embodiments, the Tₘₐₓ provided by the pharmaceutical composition containing a compound according to Formula (I), or a pharmaceutically acceptable salt thereof, and/or pridopidine, or a pharmaceutical salt thereof, is less than 0.25 hour.

In embodiments, the pharmaceutical composition containing a compound according to Formula (I), or a pharmaceutically acceptable salt thereof, and/or pridopidine, or a pharmaceutical salt thereof, provides a dissolution of at least about 80% within the first 20 minutes of administration to a subject in need thereof. In embodiments, the pharmaceutical composition containing a compound according to Formula (I), or a pharmaceutically acceptable salt thereof, and/or pridopidine, or a pharmaceutical salt thereof, provides a dissolution of at least about, e.g., 85%, 90% or 95% within the first 20 minutes of administration to a subject in need thereof. In embodiments, the pharmaceutical composition containing a compound according to Formula (I), or a pharmaceutically acceptable salt thereof, and/or pridopidine, or a pharmaceutical salt thereof, provides a dissolution of at least 80% within the first 10 minutes of administration to a subject in need thereof.

In embodiments, provided herein are methods of treating Huntington's disease including administering to a subject in need thereof or diagnosed with Huntington's disease a first pharmaceutical dosage including a sub-therapeutic amount of a compound according to Formula (I), or a pharmaceutically acceptable salt thereof and/or pridopidine, or a pharmaceutical salt thereof. In embodiments, treating Huntington's disease includes administering to a subject in need thereof a pharmaceutical composition containing a compound according to Formula (I), or a pharmaceutically acceptable salt thereof, and/or pridopidine, or a pharmaceutical salt thereof, in a sub-therapeutic amount, wherein the composition provides improvement in one or more symptoms of Huntington's disease for more than 6 hours after administration.

A sub-therapeutic dosage is an amount of a compound according to Formula (I), or a pharmaceutically acceptable salt thereof, and/or pridopidine, or a pharmaceutical salt thereof, that is less than the amount typically required for a therapeutic effect. In embodiments, a sub-therapeutic dosage is an amount of a compound according to Formula (I), or a pharmaceutically acceptable salt thereof, and/or pridopidine, or a pharmaceutical salt thereof, that alone may not provide improvement in at least one symptom of Huntington's disease but is sufficient to maintain such improvement. In embodiments, the methods provide administering a first pharmaceutical composition containing an effective amount of a compound according to Formula (I), or a pharmaceutically acceptable salt thereof, and/or pridopidine, or a pharmaceutical salt thereof, that provides improvement in at least one symptom of Huntington's disease and a second composition containing a subtherapeutic amount of a compound according to Formula (I), or a pharmaceutically acceptable salt thereof, and/or pridopidine, or a pharmaceutical salt thereof, that maintains the improvement. In embodiments, after administration of the first pharmaceutical composition, the second pharmaceutical composition may provide a synergistic effect to improve at least one symptom of Huntington's disease.

In embodiments, provided herein are methods of treating Huntington's disease including administering to a subject in need thereof or diagnosed with Huntington's disease a first pharmaceutical composition including a first pharmaceutical dosage of a compound according to Formula (I), or a pharmaceutically acceptable salt thereof, and/or pridopidine, or a pharmaceutical salt thereof, wherein the first pharmaceutical dosage provides improvement for more than 6 hours after administration, and a second pharmaceutical composition including a sub-therapeutic dosage of a compound according to Formula (I), or a pharmaceutically acceptable salt thereof and/or pridopidine, or a pharmaceutical salt thereof.

In embodiments, the first or the second pharmaceutical composition are provided to the subject once in the evening and once in the morning. In embodiments, the total amount of a compound according to Formula (I), or a pharmaceutically acceptable salt thereof, and/or pridopidine, or a pharmaceutical salt thereof, administered to a subject in a 24-hour period is any of the respective amounts described herein.

In embodiments, the first and/or the second pharmaceutical compositions may be provided with conventional release or modified release profiles. The first and second pharmaceutical compositions may be provided at the same time or separated by an interval of time, e.g., 1 hour, 2 hours, 3 hours, 4 hours, 5 hours, 6 hours, 12 hours, etc. In embodiments, the first and the second pharmaceutical compositions may be provided with different drug release profiles to create a two-phase release profile. For example, the first pharmaceutical composition may be provided with an immediate release profile, e.g., ODDF, parenteral, etc., and the second pharmaceutical composition may provide an extended release profile. In embodiments, one or both of the first and second pharmaceutical compositions may be provided with an extended release or delayed release profile. Such compositions may be provided as pulsatile formulations, multilayer tablets or capsules containing tablets, beads, granules, etc. In embodiments, the first pharmaceutical composition is an immediate release composition. In embodiments, the second pharmaceutical composition is an immediate release composition. In embodiments, the first and second pharmaceutical compositions are provided as separate immediate release compositions, e.g., film, tablets or capsules. In embodiments the first and second pharmaceutical compositions are provided 12 hours apart.

It should be understood that respective dosage amounts of a compound according to Formula (I), or a pharmaceutically acceptable salt thereof, and/or pridopidine, or a pharmaceutical salt thereof, that are provided herein are applicable to all the dosage forms described herein including conventional dosage forms, modified dosage forms, the first and second pharmaceutical compositions, as well as the parenteral formulations described herein. Those skilled in the art will determine appropriate amounts depending on criteria such as dosage form, route of administration, subject tolerance, efficacy, therapeutic goal and therapeutic benefit, among other pharmaceutically acceptable criteria.

Combination therapies utilizing a compound according to Formula (I), or a pharmaceutically acceptable salt thereof, and/or pridopidine, or a pharmaceutical salt thereof, can include administration of the active agents together in the same admixture, or in separate admixtures. In embodiments, the pharmaceutical composition can include two, three, or more active agents. In embodiments, the combinations result in a more than additive effect on the treatment of the disease or disorder. Thus, treatment is provided for Huntington's disease with a combination of agents that combined, may provide a synergistic effect that enhances efficacy.

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and/or up to 1% of a given value. Alternatively, particularly with respect to biological systems or processes, the term can mean within an order of magnitude, preferably within 5-fold, and more preferably within 2-fold, of a value.

“Improvement” refers to the treatment of Huntington's disease measured relative to at least one symptom of Huntington's disease.

“Improvement in next day functioning” or “wherein there is improvement in next day functioning” refers to improvement after waking from an overnight sleep period wherein the beneficial effect of administration of a compound according to Formula (I), or a pharmaceutically acceptable salt thereof, and/or pridopidine, or a pharmaceutical salt thereof, applies to at least one symptom of a syndrome, disease or disorder herein and is discernable, either subjectively by a subject or objectively by an observer, for a period of time, e.g., 2 hours, 3 hours, 4 hours, 5 hours, 6 hours, 12 hours, 24 hours, etc. after waking.

“PK” refers to the pharmacokinetic profile. Cₘₐₓ is defined as the highest plasma drug concentration estimated during an experiment (ng/ml). Tₘₐₓ is defined as the time when Cₘₐₓ is estimated (min). AUC_{0-∞} is the total area under the plasma drug concentration-time curve, from drug administration until the drug is eliminated (ng·hr/ml or μg·hr/ml). The area under the curve is governed by clearance. Clearance is defined as the volume of blood or plasma that is totally cleared of its content of drug per unit time (ml/min).

“Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

“Effective amount” or “therapeutically effective amount”, previously referred to, can also mean a dosage sufficient to alleviate one or more symptoms of a syndrome, disorder, disease, or condition being treated, or to otherwise provide a desired pharmacological and/or physiologic effect. “Effective amount” or “therapeutically effective amount” may be used interchangeably herein.

“Co-administered with”, “administered in combination with”, “a combination of”, “in combination with” or “administered along with” may be used interchangeably and mean that two or more agents are administered in the course of therapy. The agents may be administered together at the same time or separately in spaced apart intervals. The agents may be administered in a single dosage form or in separate dosage forms.

“Subject in need thereof” includes individuals that have been diagnosed with Huntington's disease. The methods may be provided to any individual including, e.g., wherein the subject is a neonate, infant, a pediatric subject (6 months to 12 years), an adolescent subject (age 12-18 years) or an adult (over 18 years). Subjects include mammals. “Subject” and “patient” are used interchangeably herein.

“Prodrug” refers to a pharmacological substance (drug) that is administered to a subject in an inactive (or significantly less active) form. Once administered, the prodrug is metabolized in the body (in vivo) into a compound having the desired pharmacological activity.

“Analog” and “Derivative” may be used interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

The term “pharmaceutically acceptable salt”, as used herein, refers to derivatives of the compounds defined herein, wherein the parent compound is modified by making acid or base salts thereof. Examples of pharmaceutically acceptable salts include but are not limited to mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Such conventional non-toxic salts include but are not limited to those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts. The pharmaceutically acceptable salts can be synthesized from the parent compound, which contains a basic or acidic moiety, by conventional chemical methods.

### EXAMPLES

The examples provided herein are included solely for augmenting the disclosure herein and should not be considered to be limiting in any respect.

The following Examples establish: Compound A is capable of directly binding to KCC2, as measured using cellular thermal shift assay (CETSA). Compound A potently increases KCC2 activity without modifying its plasma membrane stability or the phosphorylation of key regulatory sites, as measured by patch-clamp recording and TI flux. This contrasts to other indirect potentiators of KCC2, which have been shown to increase expression levels on the plasma membrane, enhance its expression, increase transcription of the SLC12A5 gene, or modify its phosphorylation (Gagnon et al., Nat. Med. 19, 1524-1528 (2013); Lee et al., Brain: a journal of neurology 145, 950-963 (2022); Prael Iii et al., Front Cell Dev Biol 10, 912812 (2022); Tang et al., Sci. Transl. Med. 11. 10.1126) (2019)). Consistent with its role in limiting neuronal excitability, Compound A reduced neuronal Cl⁻ accumulation and the development of late recurrent discharges (LRDs) in acute brain slices exposed to 0-Mg. Compound A rapidly accumulated in the brain when dosed IV, IP, or SC to concentrations ranging from 40-0.6 μM, dependent upon the route of administration. Importantly, as measured in the open field, Compound A appeared to have no gross effects on mouse behavior.

The Examples establish that in mice treated with Compound A and diazepam, neuronal cell death in the hippocampus was significantly reduced compared to controls treated with diazepam alone. This effect may be due to the ability of Compound A to limit hyperexcitability, but previous in vitro and in vivo studies have shown that reducing KCC2 expression levels, or transiently inhibiting it, are sufficient to induce neuronal apoptosis. Likewise, modifying KCC2 expression levels during development also impacts neuronal viability.

### Example 1

Identification of Small Molecules that Activate and Directly Bind to KCC2

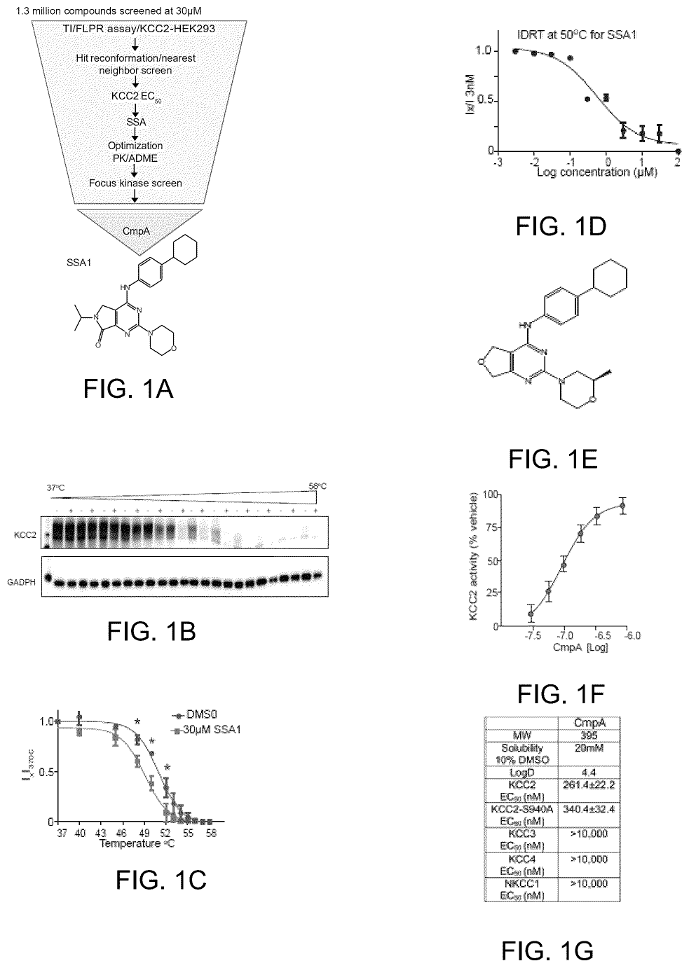

A multi-tiered high-throughput screening assay was used, leveraging a proprietary library of 1.3 million small molecule compounds from AstraZeneca (Cambridge, UK) to identify chemical entities capable of activating KCC2. A single point concentration of 30 μM was used to identify potassium flux using an established thallium (TI+) influx FLIPR assay as the primary read out in HEK-293 cells overexpressing KCC2 (Cardarelli et al., Nat. Med. 23, 1394-1396 (2017); Conway et al., J Biol Chem 292, 21253-21263 (2017); Lee et al., 2022, supra). Compounds that exhibited >20% potentiation of KCC2 activity with EC₅₀ of <30 μM were selected for further optimization. This approach resulted in the identified subseries of fused pyrimidine compounds as KCC2 activators, which are denoted as subseries A (SSA; FIG. 1A).

Neuronal Cultures. Hippocampal neurons were prepared from Sprague Dawley E18 embryos and plated at 450,000 per dish and maintained at 37° C. in a humidified 5% CO₂ incubator for 10-25 days before experimentation (Lee et al., Nat. Neurosci. 14, 736-743 (2011)).

Cell Culture and biotinylation. HEK-293 cells were purchased from ATCC (CRL-1573) and transfected with human KCC2 and GFP expression plasmids using Lipofectamine 2000. Cells were utilized for experimentation 48-72 h following transfection. Cells were biotinylated at 4° C. using NHS-Biotin. Following lysis and purification on immobilized avidin beads, cell surface and total fractions were subject to immunoblotting as outlined previously (Conway et al., 2017, supra).

FLIPR TI Flux assay. HEK-293 cells were washed with HBSS, and then incubated with loading buffer containing TI sensitive fluorescent dye (https://www.moleculardevices.com), 20 μM bumetanide 20 μM ouabain and 2.5 mM probenecid for 1 h at room temperature (RT) under control conditions or with drugs in the dark using a FlexStation 3 microplate reader (Molecular Devices) and imaged 525 nM for 30 min. Stimulus buffer was added containing 5 mM K⁺/0.5 mM Tl⁺ and cells were imaged every 1.5 over a 190 second time course The initial rate Tl⁺ uptake (Relative Fluorescence Units (RFU) per second) was determined following addition of the stimulus buffer as outlined previously (Conway et al., 2017, supra) (Lee et al., 2022, supra).

### Example 2

### Identification of Compound A

Determining if compounds in SSA directly interact with KCC2. This was tested using a cellular thermal shift assay (CETSA), which measures the ability of a drug to modify the thermal stability of its target in whole cells (Kawatkar et al., ACS Chem. Biol. 14, 1913-1920 (2019); Martinez et al., Sci Rep 8, 9472 (2018)).

2×10⁶ transfected HEK-293 cells were incubated with 30 μM drug or vehicle for 90 min at 37° C. and then heated from 37-58° C. for 5 min. Cells were rapidly chilled to 25° C. and NP40 was added to a final concentration of prior to snap freezing in liquid nitrogen. Following 5 freeze/thaw cycles lysates were centrifuged at 12,000×g to remove denatured proteins and associated cell debris. The supernatant cell and tissue lysate samples, or immunoprecipitated KCC2 samples were separated by SDS-PAGE and subsequently immunoblotted with KCC2 antibodies (1:2000, Millipore #07-432), KCC2-S940, KCC2-pT1007 (1:2000, PhosphosSolutions, #1568 actin and GAPDH (1:3000, SantaCruz #sc-32233) as detailed in previous studies (Lee et al., 2011, supra) (Conway et al., 2017, supra), and the level of soluble KCC2 at each temperature was normalized to that seen at 37° C. This data was then used to determine the temperature at which 50% of KCC2 was denatured.

Aliquots of HEK-293 cells expressing KCC2 were exposed to 30 μM SSA, heated from 37-55° C. The remaining level of soluble native KCC2 at each temperature was measured by immunoblotting and normalized to the level seen at 37° C. which was given a value of 1. SSA-1 reduced the thermal stability of KCC2 compared to vehicle, which demonstrated target engagement. To gain insights into affinity, isothermal dose response analysis was performed at 50° C. revealing a K_{D} of 395.6±23.5 nM of SSA for KCC2 (FIG. 1B-D). SSA derivatives were further optimized for affinity, optimal drug like properties and predicted brain penetration by in Vitro cellular uptake assays, which resulted in the identification of Compound A (FIG. 1E). Dose response measurements with Compound A were then made at 50° C. and this data was used to estimate its relative affinity for KCC2 (Kawatkar et at, 2019, supra) (Martinez et al., 2018, supra). To control for compounds that may indirectly modulate KCC2 activity, via modifying its phosphorylation (Kahle et al., Trends Neurosci. 36, 726-737 (2013); Moore et al., Proc. Natl. Acad. Sci. U.S.A. 115, 10166-10171 (2018); Silayeva et al., Proc. Natl. Acad. Sci. U.S.A. 112, 3523-3528 (2015); Smalley et al., Front. Mol. Neurosci. 13, 563091 (2020)), the effect of Compound A to modify the activity of a panel of 144 protein kinases (SelectScreen; Thermofisher) was studied. This panel included protein kinase C isoforms, OSR1, SPAK (STK39) and with-no-lysine kinases (WNK), all of which phosphorylate KCC2 on multiple residues to indirectly modify its activity (Kahle et al., 2013, supra; Smalley et al., 2020, supra). Compound A did not modify the activity of this kinase panel.

As measured by TI+ flux, Compound A exhibited an EC₅₀ for KCC2 of 261.4±22.2 nM and a maximum potentiation of 90% at a saturating concentration of 3 μM using a 1% β-cyclodextrin (BCD) vehicle (FIG. 1F). Several studies have shown that the activity and plasma membrane accumulation of KCC2 is potentiated via phosphorylation of S940, a process mediated by the activity of protein kinase C and protein phosphatase 1 (Lee et al., 2011, supra; Lee et al., J Biol Chem 282, 29777-29784 (2007); Moore et al., Proc. Natl. Acad. Sci. U.S.A. 115, 10166-10171 (2018); Moore et al., Trends Neurosci. 40, 555-571 (2017); Silayeva et al., Proc. Natl. Acad. Sci. U.S.A. 112, 3523-3528 (2015)). The ability of Compound A to modulate the activity of a KCC2 construct was tested in which this key residue for phospho-dependent modulation of KCC2 was mutated to an alanine (S940A). Significantly, Compound A increased the activity of KCC2-S940A construct (FIG. 1G). Finally, the specificity of Compound A for other solute transporters was examined using the TI+ assay using HEK-293 cells expressing KCC3, KCC4, or NKCC1. Compared to KCC2, effects on KCC3, KCC4, and NKCC1 were only seen at concentrations more than 35-fold that of KCC2 (>100 μM) (FIG. 1G).

These results suggest that Compound A binds directly to KCC2 and acts to increase its activity with minimal effects on other solute transporters that regulate neuronal Cl⁻ accumulation.

### Example 3

Compound a Potentiates KCC2 Activity without Modifying its Plasma Membrane Accumulation or Phosphorylation

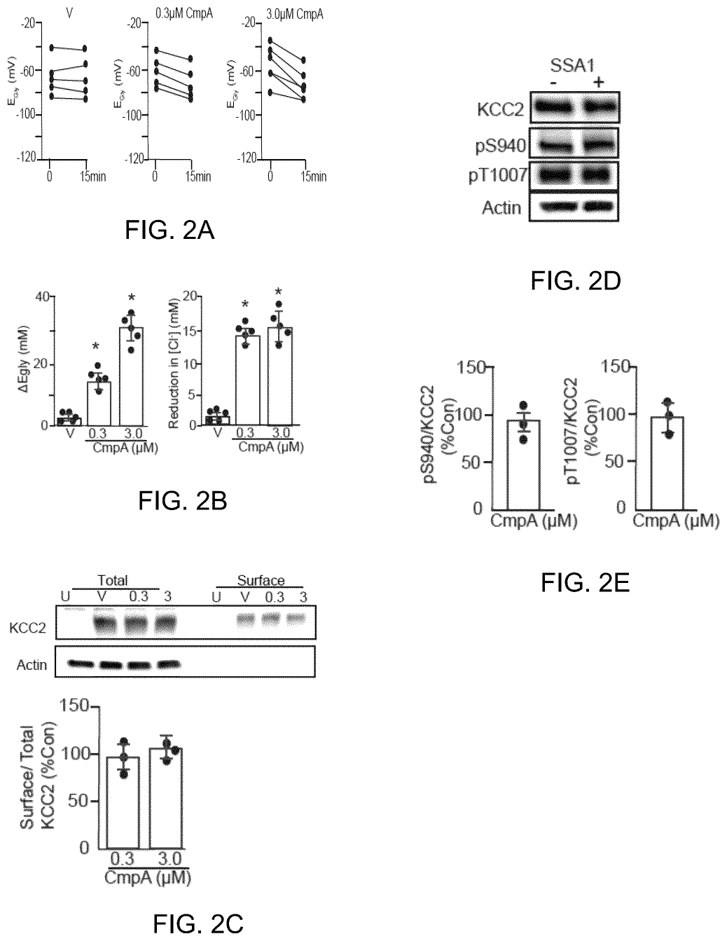

To confirm the validity of our measurements using TI+ flux, KCC2 was expressed in HEK-293 cells with the al subunit of the glycine receptor (GlyRα1) which forms homomeric glycine-activated ion channels when expressed in this system. The gramicidin perforated patch clamp technique was used to measure the reversal potential for GlyRα1-mediated currents (E_{GLY}) in cells exposed to Compound A. The use of this technique, while limited in throughput, facilitates high resolution single-cell recordings without disturbing endogenous intracellular Cl⁻ levels (Cardarelli et al., 2017, supra; Conway et al., 2017, supra). To calculate E_{GLY}, cells were exposed to 50 μM Gly and the polarity of Gly-induced currents was determined at different holding potentials. Brief (15 min) incubation with either 0.3 μM (approx. EC₅₀) or 3 μM Compound A induced shifts of −15.6±4.5 (p=0.022), and −32.2±9.5 (p=0.001) mV in E_{GLY} respectively, while vehicle was without effect (−0.8±0.5 mv; p=0.512) (FIG. 2A-B). As determined using the Nernst equation, these negative shifts in E_{GLY} by Compound A corresponded to reduced intracellular Cl⁻ levels of FIG. 2B; Compound A; 5.2±1.2; vehicle=7.9±1.2 mM; p=0.0104).

The effects of Compound A on the plasma membrane stability of KCC2 expression was assessed using biotinylation, as described previously (Cardarelli et al., 2017, supra; Conway et al., 2017, supra). Compared to vehicle, 0.3 μM or 3 μM Compound A did not modify the level of KCC2 on the plasma membrane (FIG. 2C; 94.5±8.4%, p=0.745 and 104.7±8.4% of control; p=0.736 respectively). The reliability of the biotinylation procedure was assessed via immunoblotting with antibodies against the cytosolic protein actin. Actin was present in the total lysates but was absent from the surface fractions (FIG. 2C).

The activity of KCC2 is subject to dynamic modulation via phosphorylation of critical intracellular residues within its C-terminal cytoplasmic domain. Central to this process are serine 940 (S940) and threonine 1007 (T1007), which activate and inhibit KCC2 activity respectively (Moore et al., 2017, supra). Therefore, the effects of Compound A on the phosphorylation of these residues was examined using immunoblotting with characterized phospho-specific antibodies pS940 and pT1007, respectively. Compound A (3 μM) did not significantly modify phosphorylation of S940 (95.3±10.2% of control; p=0.764), or T1007 (108±10.2% of control; p=0.649) (FIG. 2D).

The foregoing suggests that Compound A potentiates KCC2 activity without modifying either its plasma membrane accumulation or the phosphorylation of key regulatory sites.

### Example 4

### Compound a Reduces Neuronal Cl⁻ Accumulation and Slows the Development of Seizure-Like Events n In Vitro

To test if Compound A modifies neuronal Cl⁻ levels gramicidin-perforated patch-clamp recording was used to measure the reversal potentials of GABA_{A} receptor mediated currents (E_{GABA}) in 18-20 Div hippocampal neurons (Lee et al., 2011, supra; Moore et al., 2018, supra). At this developmental stage, GABA_{A}R receptor activation leads to hyperpolarization, a phenomenon critically dependent upon KCC2 (Kontou et al., J Biol Chem, 100364 (2021)).

Neuronal Patch clamp assays. Neuronal cell culture recordings were performed in bath saline at 34° C. For gramicidin perforated-patch experiments, pipettes contained (in mM): 140 KCl and 10 HEPES, pH 7.4 KOH. For whole-cell experiments, pipettes contained (in mM): 115 K-meth-SO₄, 30 KCl, 2 Mg-ATP, 4 Na-ATP, 0.4 Na-GTP, and 10 HEPES, pH 7.4 KOH. Bath saline contained the following (in mM): 140 NaCl, 2.5 KCl, 2.5 CaCl₂, 2.5 MgCl₂, 10 HEPES, and 11 glucose, pH 7.4 NaOH (Kontou et al., 2021, supra). All compound solutions were applied to cells using a three-barreled 700 μm pipe positioned just above the cell (Warner Instruments). Compound A was applied in 1 mg/mL 2-Hydroxypropyl-β-cyclodextrin, and its effects were compared to this vehicle alone. 10 mV voltage-ramp protocols over 1-s periods were used to determine the reversal potentials of the leak-subtracted muscimol-activated GABA_{A}R currents. All voltages from whole-cell experiments were corrected offline using a calculated liquid junction potential value (13.6 mV) in Clampex (Molecular Devices). For gramicidin perforated-patch experiments, intracellular Cl⁻ values were back-calculated using measured E_{GABA} values and the Nernst equation:

$${E}_{ion} = \frac{RT}{zF} ln \frac{{[ ion ]}_{o}}{{[ ion ]}_{i}}$$

where E is the ion's reversal potential, R is the universal gas constant, T is temperature in kelvins, F is Faraday's constant, z is the charge of the ion, [chloride]ᵢ is the intracellular chloride concentration and [chloride]ₒ is the extracellular ion concentration.

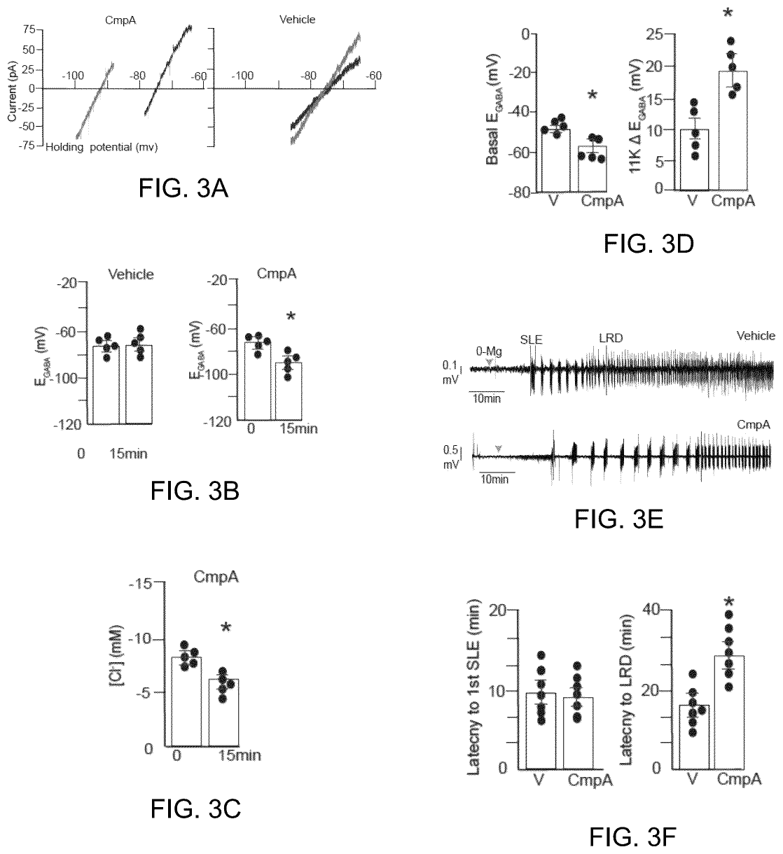

Measurements were performed in the presence of bumetanide (10 μM) and tetrodotoxin (TTX; 300 nM) to limit the contributions of NKCC1 and activity dependent shifts in chloride levels, respectively (Lee et al., 2011, supra). Cultures were exposed to Compound A or vehicle for 15 min and voltage ramps were used to determine changes in the reversal potential of GABA_{A}R receptor currents over time (FIG. 3A). These data were then used to determine shifts subsequently, using [Cl⁻] levels. At 15 min 300 nM Compound A (an approximately EC₅₀ concentration) significantly reduced E_{GABA} from −75±3.1 to −85.1±4.2 mV (FIG. 3B; p=0.0104), a net −9.1±2.2 mV negative shift, reflecting a 2.2±0.6 mM reduction in Cl⁻ (FIG. 3C; p=0.0153). In contrast, vehicle did not significantly modify E_{GABA} (FIG. 3B; 0=−77.2±3.3, 15=−78.0±4.3 mV respectively, p=0.580) or chloride levels.

To measure the effects of Compound A on KCC2 activity under more dynamic conditions cultures in Compound A were incubated for 1 h and subjected to whole cell patch-clamp recording to artificially impose a 32 mM Cl⁻ load on neurons via the patch pipette (Lee et al., 2022, supra; Moore et al., 2018, supra). 5 min after break-in, an initial E_{GABA} value was determined and the effects of a subsequent 5 min incubation with VU0463271 was assessed. Compound A-treated cells exhibited lower basal E_{GABA} values compared to controls (FIG. 3C; FIG. 3D −59.3±2.1 and −47.4±2.3 mv, respectively, p=0.025). Likewise, VU0463271 induced shifts in E_{GABA} were larger for cultures treated with Compound A (FIG. 3C; FIG. 3D 17.5±4.6 and 5.4±4.2 mV, respectively, p=0.009).

The effects of Compound A were assessed on the development of seizure like events (SLE) in brain slices exposed to ASCF deficient in Mg⁺² (0-Mg), a widely used method to increase neuronal excitability (Anderson et al., Brain research 398, 215-219 (1986)). In vitro seizure assays. Horizontal/Coronal slices (400 μM) are prepared from 5-7 week old male mice and placed in a submerged chamber for a 60-min recovery period at 32° C. in normal ACSF as detailed previously (Kelley et al., Neuropharmacology 108, 103-110 (2016); Moore et al., 2018, supra). Slices were then transferred to a Warner Instruments recording chamber and electrodes of 0.5-1 mohm were inserted into layer III/IV of the medial entorhinal cortex, several cell layers deep. Slices were perfused for 10 min with normal ACSF before exposure to media deficient in Mg⁺² supplemented containing vehicle (1 mg/mL 2-Hydroxypropyl-β-cyclodextrin), or Compound A. Recordings were made within the entorhinal cortex with a Multiclamp 700B amplifier (Molecular Devices) with Clampex 10 acquisition software (Molecular Devices. The time spent in seizure-like activity was measured using threshold detection software in Clampfit. 3×the standard deviation of a 1 min period of the baseline immediately prior to/0-Mg2+ onset was used as the cut off for event detection. The duration between each above-threshold event was calculated, and inter-event durations of <20 s were considered as a continuation of the same seizure-like event. The field recordings were then employed to monitor the development of seizure-like events (SLEs) and their transition to late recurrent discharges (LRDs) which are believed to be analogous to the development of status epilepticus (SE) (Kelley et al., 2016, supra; Moore et al., 2018, supra). Relative to control, Compound A modified the appearance of the first SLE relative to control (FIG. 3E, F; Compound A=11.3±3.3 min, and vehicle=10.5±2.6 min, respectively, p=0.256). In addition, Compound A slowed the development of LRDs Cellular Thermal Shift Assay (FIG. 3E, F; Compound A=28.4±6.4 min, vehicle=12.4±4.1 min, p=0.010).

Thus, Compound A reduces neuronal Cl⁻ levels and slows the development of hyperexcitability in an ex vivo system.

### Example 5

### Compound A Rapidly Accumulates in the Brain and Protects Against Pentylenetetrazol (PTZ) Induced Convulsions In Vivo

PK studies. Mice were injected intraperitoneally (IP), intravenously (IV), or subcutaneously (SC), with Compound A and 5% BCD in a maximum volume of 300 μl. Plasma and brain samples were rapidly frozen on dry ice. Tissue extracts were treated with Acetonitrile and Compound A levels were quantified using LC-MS/MS (Integrated Analytical Solutions, Inc, Berkeley, CA 94710; www.ianalytical.net).

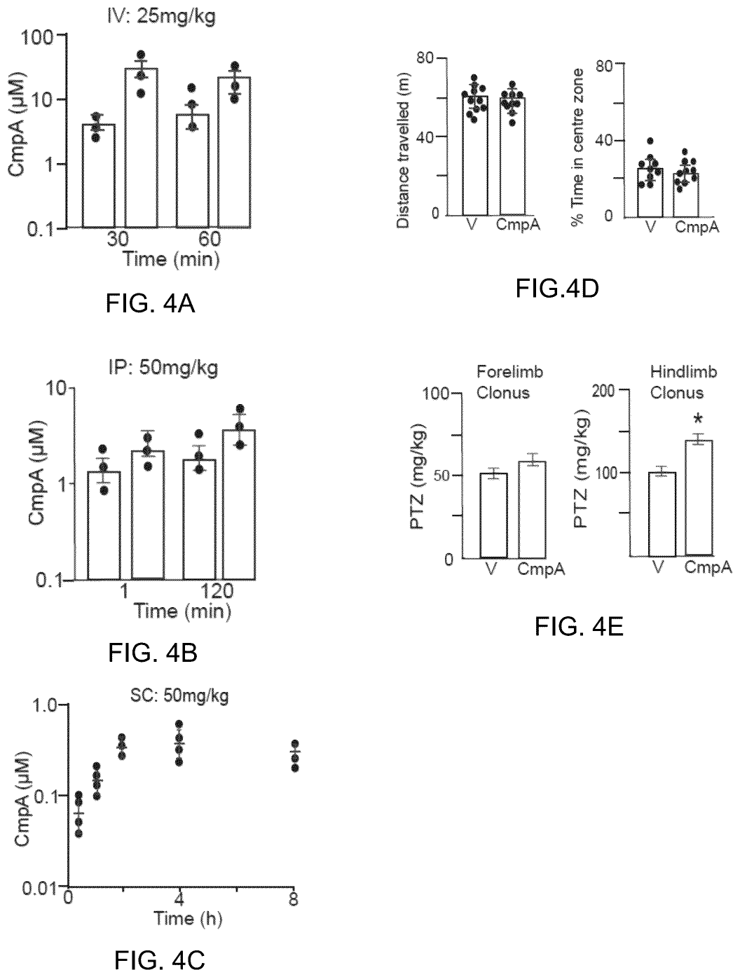

To examine the ability of Compound A to cross the blood brain barrier, mice were injected with a single 25 mg/kg IV bolus; accumulation in the brain was evident within 30 minutes min in the brain and plasma to 7.6±4.4 and 41.5±11.5 and 11.2 μM respectively (FIG. 4A). Likewise, a 50 mg/kg IP injection resulted in the accumulation of Compound A in the brain to 2.67±0.21 and 4.1±0.13 μM 2 and 4 hr later (FIG. 4B). Drug accumulation in the brain was examined following SC injection (FIG. 4C; 50 mg/kg). Accumulation was detected at 30 min and reached a maximal concentration of 675.75±80.5 nM at 4 hr, a level that was maintained 8 h later (FIG. 4C).

Given the ability of Compound A to accumulate in the brain, it was examined to see if it also causes any gross effects on animal behavior. Mice were injected with Compound A (SC; 50 mg/kg), or vehicle and their behavior was compared in the open field test (Tretter et al., Proc. Natl. Acad. Sci. U.S.A. 106, 20039-20044 (2009); Vien et al., Frontiers in molecular neuroscience 15, 817996 (2022)). Compound A did not modify the total distance traveled during the test trial (FIG. 4D; Compound A=6134.2±235.3 cm, and vehicle=6453.7±215.4 cm; p=0.253, n=11 mice). Likewise, time spent in the center zone was comparable (FIG. 4D; Compound A=18.4±2.1, and vehicle 16.6±2.5% of total time; p=0.658, n=11 mice).

Collectively, these results suggest that Compound A accumulates in the brain when dosed IV, IP or SC, and does not lead to any stereotyped effects on behavior.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

To gauge if Compound A exerts any anticonvulsant efficacy its ability to protect against motor seizures induced by the convulsant (PTZ), a GABA_{A}R antagonist (Shimada and Yamagata, J Vis Exp. 10.3791/56573 (2018)) was determined. To do this, mice were dosed IV with 12.5 mg/kg Compound A or vehicle, and then the modified Racine scale was used to measure the dose of PTZ required to induce forelimb and hindlimb clonus, stage 3 and 4/5 seizures, was assessed (Van Erum et al., Epilepsy Behav 95, 51-55 (2019)). While Compound A did not protect against forelimb clonus (FIG. 4E; Control=51.5±3.1, Compound A=56.3±3.3 mg/kg; p=0.120, n=14 mice), the dose of PTZ required to induce hindlimb clonus was significantly increased by Compound A pretreatment (FIG. 4E; Control=84.5±5.1 mg/kg, Compound A=137.4±7.5 mg/kg respectively, p=0.001, n=11-14 mice).

Collectively, these results suggest that Compound A accumulates in the brain when dosed IV, IP or SC, and does not lead to any stereotyped effects on behavior but does protect against late-stage seizures induced by PTZ, a pharmacological screening tool widely used to identify anticonvulsants.

### Example 6

### KCC2 Activation Reduces the Severity of SE and the Development of BDZ Resistance, In Vivo

To further explore the anticonvulsant properties of Compound A, its effects on the development of kainic acid (KA) induced SE in mice was examined using electroencephalographic (EEG) recording (Silayeva et al., 2015, supra). This model was chosen due to the similarities with patients undergoing SE, as KA-induced seizures become refractory to benzodiazepines (BDZs) within minutes (Betjemann and Lowenstein, Lancet Neurol 14, 615-624 (2015); Crawshaw and Cock, (2020a) [Seizure: Eur. J. Epilep. 75 (2020) 145-152] Seizure 80, 282; Lee et al., 2022, supra; Reddy and Kuruba, Int J Mol Sci 14, 18284-18318 (2013)).

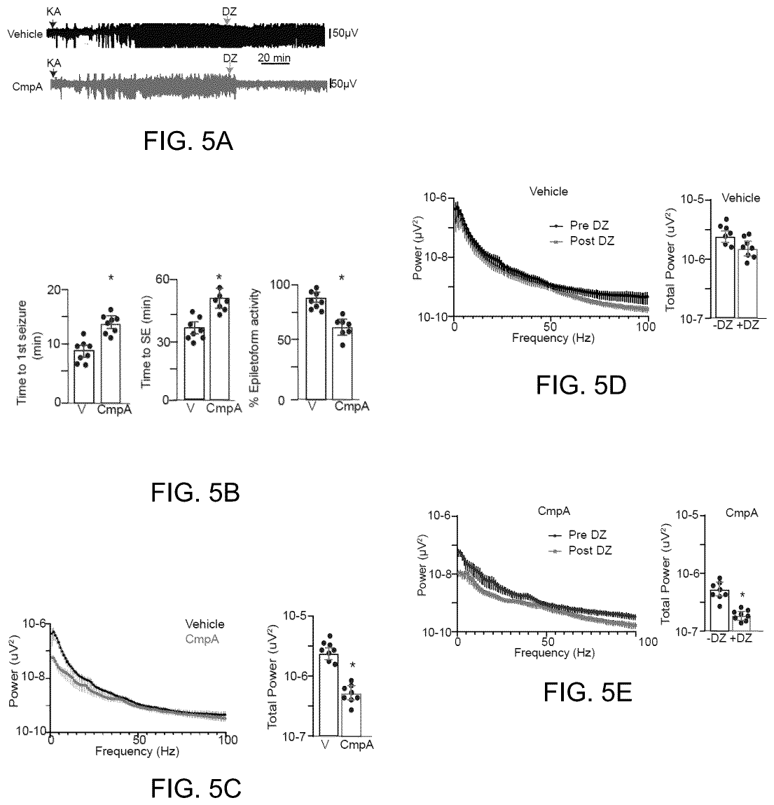

EEG recordings and analysis. WT C57Bl/6J adult male mice (12 weeks) were anesthetized with isofluorane and implanted with EEG/EMG electrodes and allowed to recover for 7 days prior to experimentation. For pre-dosing basal EEGs were recorded for 30 min prior to injection with 50 mg/kg Compound A (SC), or vehicle followed 2 h later with 20 mg/kg KA (IP). 2 h following KA administration mice were injected with a saturating concentration of diazepam (DZ) 10 mg/kg (IP) and recordings were extended for a further 60 min. Alternatively basal EEGs were recorded for 30 min prior to dosing with 20 mg/kg KA, followed 2 h later with 10 mg/kg DZ alone or together with 50 mg/kg Compound A (IP) and recordings were extended for a further 60 min (FIG. 5A). LabChart 8 software was used for data analysis. Epileptiform activity was identified by changes in the amplitude of electrographic activity characterized by consistent changes in the power of the Fast Fourier transform (FFT) of the EEG and abnormal activity characterized by periods of rhythmic spiking lasting longer than 30 s (Moore et al., 2017, supra; Silayeva et al., 2015, supra; Sivakumaran et al., J. Neurosci. 35, 8291-8296 (2015)).

First, the effects of Compound A on the development of SE were examined, revealing increased latency to the first seizure (FIG. 5B; vehicle=10.2±2.5 min, Compound A=14.4±2.9 min, p=0.0329, n=10 mice) and to SE, compared to vehicle (FIG. 5B; vehicle=38.3±3.4 min, and Compound A=54.5±6.9 min, p=0.0230, n=10 mice). In addition, the time spent in epileptiform activity was lower in mice pretreated with Compound A (FIG. 5B; vehicle=85±5.4%, and Compound A=62.5±0.8.4%, p=0.0230, n=9 mice).

To provide quantitative insights on the effects of KA, raw EEG recordings were subject to Fast-Fourier transformation (8K FFT size, Hann (cosine-bell), 87.50% window overlap) for frequencies between 0-100 Hz 2 h after KA injection (Lee et al., 2022, supra). In mice pretreated with Compound A total EEG power was significantly reduced compared to vehicle (FIG. 5C; 4.2±2.4×10⁻⁶ and 6.9±3.2×10⁻⁷ μV² for vehicle and Compound A, respectively; p=0.0024, n=11 mice).

Finally, the effects of DZ on EEG power were compared min prior to and following DZ treatment. In mice pretreated with Compound A, DZ significantly reduced EEG power (FIG. 5D; 6.9±3.2×10⁻⁷ and 2.7±3.2×10⁻⁷ μV², pre and post DZ respectively; p=0.004, n=9 mice). In contrast, but consistent with previously published studies on the KA model, DZ did not modify EEG power in mice pretreated with vehicle (FIG. 5D; 4.2±2.4×10⁻⁶ and 2.9±2.7×10⁻⁶ μV² pre and post DZ respectfully; p=0.105).

To confirm that Compound A acts centrally to mediate its effects on seizure activity, Compound A (10 μM), (20 μM) or vehicle was injected directly into the hippocampus of mice implanted with EEG/EMG electrodes (Lee et al., 2022, supra). 30 or 60 min later mice were injected with KA (20 mg/kg) and 2 h later with DZ as detailed above. Relative to vehicle Compound A increased the latency to the 1^{st} seizure, to SE, and reduced the percentage of time in epileptiform activity. 2 h after KA injection DZ did not significantly modify EEG power in mice treated with vehicle. However, DZ significantly reduced seizure parameters in mice pretreated with Compound A, thus restoring the efficacy of BDZs in terminating RSE.

Collectively, these observations demonstrate that pre-treatment of mice with Compound A reduces the severity of KA induced SE and restores BZD efficacy, preventing the development of RSE.

### Example 7

### Compound a Restores the Ability of DZ to Arrest Ongoing SE and Limits Subsequent Neuronal Cell Death

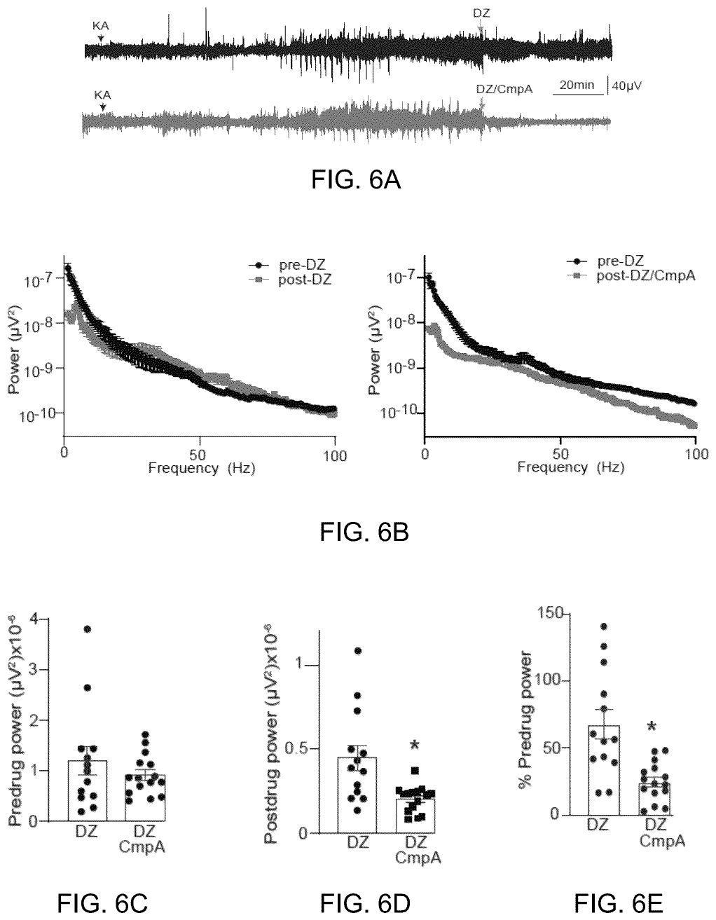

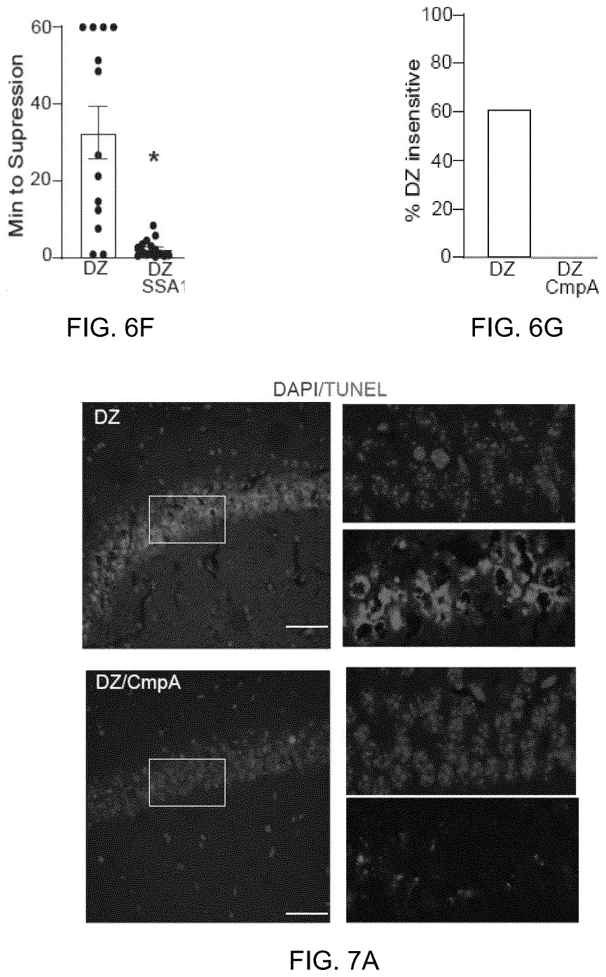

To evaluate the potential of KCC2 activators to terminate ongoing SE, the KA model described above was employed. Mice were injected with KA (20 mg/Kg; IP). 2 h later mice were subsequently dosed with DZ alone or DZ/Compound A IP (DZ=10 mg/kg; Compound A=50/mg/Kg), and recordings were extended for a further 60 min (FIG. 6A). Spectral data were then subjected to FFT and the total EEG power between 1-100 Hz of the 2 h following KA treatment (pre-drug) and 30 min following their injection (post-drug) were compared (FIG. 6B). In mice treated with DZ alone variable effects were observed, from large increases to modest reductions in EEG power, but these effects were not significant (FIG. 6C; pre=1.1±0.2×10⁻⁶, post=4.4±7.4×10⁻⁷ μV², p=0.214, n=13-14 mice). In contrast, total EEG power was significantly reduced in mice treated with DZ/Compound A (FIG. 6D; pre=9.2±1.0×10⁻⁷, post=2.0±0.2×10⁻⁷ μV², p=0.007, n=13-15 mice) reflecting a decrease in power of approximately 72%. The latency of the treatments to modify EEG power was examined by measuring the time taken for this parameter to be reduced to 50% (FIG. 6E). The mean time to suppression in DZ/Compound A treated mice was 2.3±0.5, compared to 32.5±6.7 min in those treated with DZ alone (FIG. 6F, G; p=0.0008, n=13-14 mice). Finally in mice treated with DZ alone 62% were insensitive to the effects of this agent on EEG power. In contrast, DZ insensitivity was abolished by Compound A (FIG. 6G).

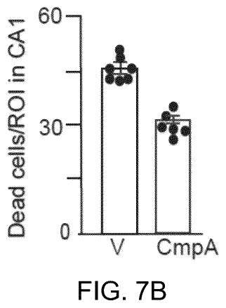

KA-induced seizures lead to neuronal cell death in many brain regions 48-72 h after treatment (Ben-Ari and Cossart, Trends Neurosci. 23, 580-587 (2000)). To determine if KCC2 activators have an impact on the extent of neuronal injury, brain sections from mice that survived for 48 h after KA injection were subjected to Terminal deoxynucleotidyl transferase dUTP nick end (TUNEL) staining to visualize dead cells, and counterstained with DAPI. The number of TUNEL positive cells in the CA1 region of the hippocampus were then compared between treatment groups. Consistent with published studies, high levels of TUNEL positive cells were found in mice injected with DZ alone. However, DZ/Compound A treatment reduced the number of TUNEL positive CA1 cells (FIG. 7A, 7B; DZ=40.93±3.35 and DZ/Compound A=31.07±2.64 dead cells/ROI, p=14 stacks, from 4 mice).

These results demonstrate that Compound A restores the ability of DZ to arrest ongoing SE and limits subsequent neuronal cell death.

It should be understood that the examples and embodiments provided herein are exemplary examples and embodiments. Those skilled in the art will envision various modifications of the examples and embodiments that are consistent with the scope of the disclosure herein. Such modifications are intended to be encompassed by the claims.

What is claimed is:

1. A method of treating Huntington's disease comprising administering a compound according to Formula (I), 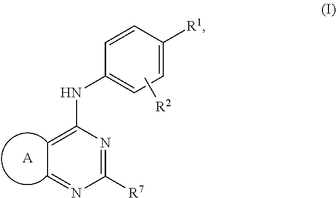
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 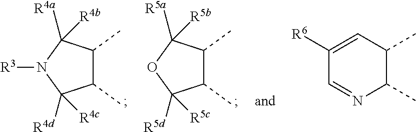
or a N-oxide thereof;
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₜR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
in combination with pridopidine, or a pharmaceutically acceptable salt thereof, to a subject diagnosed with Huntington's disease.

2. The method of treating Huntington's disease according to claim 1, wherein the total amount of the compound according to Formula (I), or a pharmaceutically acceptable salt thereof, administered to the subject is between about 1 mg and about 1500 mg.

3. The method of treating Huntington's disease according to claim 1, wherein the compound according to Formula (I), or a pharmaceutically acceptable salt thereof, is Compound A:

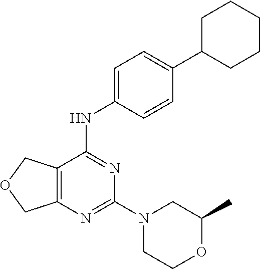 [SMILES: C[C@@H]1CN(c2nc3c(c(Nc4ccc(C5CCCCC5)cc4)n2)COC3)CCO1]

4. The method of treating Huntington's disease according to claim 1, wherein the compound according to Formula (I), or a pharmaceutically acceptable salt thereof, is Compound B:

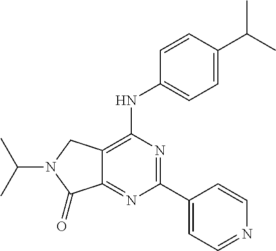 [SMILES: CC(C)c1ccc(Nc2nc(-c3ccncc3)nc3c2CN(C(C)C)C3=O)cc1]

5. The method of treating Huntington's disease according to claim 1, wherein the compound according to Formula (I), or a pharmaceutically acceptable salt thereof, is Compound C:

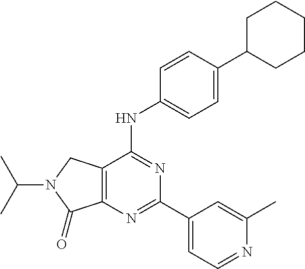 [SMILES: Cc1cc(-c2nc(Nc3ccc(C4CCCCC4)cc3)c3c(n2)C(=O)N(C(C)C)C3)ccn1]

6. The method of treating Huntington's disease according to claim 1, wherein the compound according to Formula (I), or a pharmaceutically acceptable salt thereof, is Compound D:

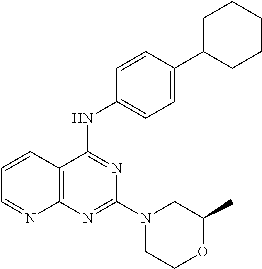 [SMILES: C[C@@H]1CN(c2nc(Nc3ccc(C4CCCCC4)cc3)c3cccnc3n2)CCO1]

7. The method of treating Huntington's disease according to claim 1, wherein the compound according to Formula (I), or a pharmaceutically acceptable salt thereof, is Compound E:

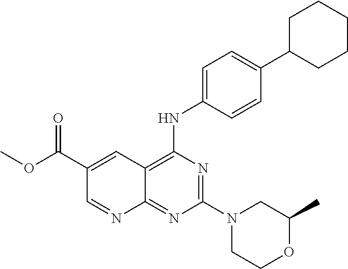 [SMILES: COC(=O)c1cnc2nc(N3CCO[C@H](C)C3)nc(Nc3ccc(C4CCCCC4)cc3)c2c1]

8. The method of treating Huntington's disease according to claim 1, wherein the compound according to Formula (I), or a pharmaceutically acceptable salt thereof, is Compound F:

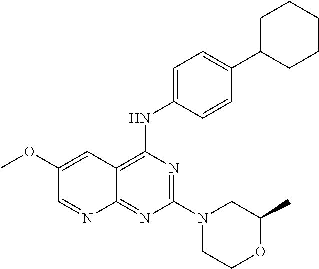 [SMILES: COc1cnc2nc(N3CCO[C@H](C)C3)nc(Nc3ccc(C4CCCCC4)cc3)c2c1]

9. The method of treating Huntington's disease according to claim 1, wherein the compound according to Formula (I), or a pharmaceutically acceptable salt thereof, is Compound G:

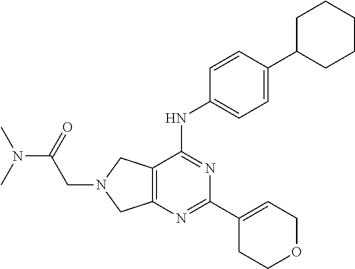 [SMILES: CN(C)C(=O)CN1Cc2nc(C3=CCOCC3)nc(Nc3ccc(C4CCCCC4)cc3)c2C1]

10. The method of treating Huntington's disease according to claim 1, wherein the compound according to Formula (I), or a pharmaceutically acceptable salt thereof, is Compound H:

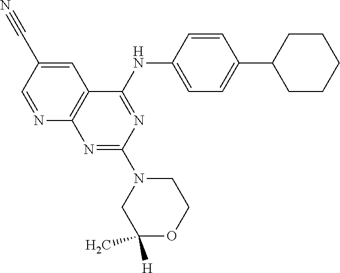 [SMILES: C[C@@H]1CN(c2nc(Nc3ccc(C4CCCCC4)cc3)c3cc(C#N)cnc3n2)CCO1]

11. The method of treating Huntington's disease according to claim 1, wherein the amount of pridopidine, or a pharmaceutically acceptable salt thereof, administered to the subject is from about 0.1 mg to about 1000 mg.

12. The method of treating Huntington's disease according to claim 1, wherein the compound according to Formula (I), or a pharmaceutically acceptable salt thereof, or pridopidine, or a pharmaceutically acceptable salt thereof, is administered enterally.

13. The method of treating Huntington's disease according to claim 12, wherein the compound according to Formula (I), or a pharmaceutically acceptable salt thereof, or pridopidine, or a pharmaceutically acceptable salt thereof, is administered orally, sublingually, buccally, transdermally or rectally.

14. The method of treating Huntington's disease according to claim 1, wherein the compound according to Formula (I), or a pharmaceutically acceptable salt thereof, or pridopidine, or a pharmaceutically acceptable salt thereof, is administered parenterally.

15. The method of treating Huntington's disease according to claim 1, wherein the compound according to Formula (I), or a pharmaceutically acceptable salt thereof, or pridopidine, or a pharmaceutically acceptable salt thereof, is administered in the form of a liquid for parenteral use, a tablet, a capsule, a caplet, a pill, an oral liquid, a lozenge, a film, a powder, an aerosol, or a patch.

16. The method of treating Huntington's disease according to claim 1, wherein the compound according to Formula (I), or a pharmaceutically acceptable salt thereof, or pridopidine, or a pharmaceutically acceptable salt thereof, is administered as an extended release dosage form.

17. The method of treating Huntington's disease according to claim 1, wherein the compound according to Formula (I), or a pharmaceutically acceptable salt thereof, or pridopidine, or a pharmaceutically acceptable salt thereof, is administered as an instant release dosage form.

18. The method of treating Huntington's disease according to claim 1, wherein the compound according to Formula (I), or a pharmaceutically acceptable salt thereof, or pridopidine, or a pharmaceutically acceptable salt thereof, is administered as a delayed release dosage form.

19. The method of treating Huntington's disease according to claim 1, wherein the subject is a human subject.

20. The method of treating Huntington's disease according to claim 1, wherein the method of treating the subject diagnosed with Huntington's disease improves a symptom selected from the group consisting of motor disturbances, cognitive disturbances, and psychiatric disturbances.

21. A pharmaceutical composition comprising a compound according to Formula (I), 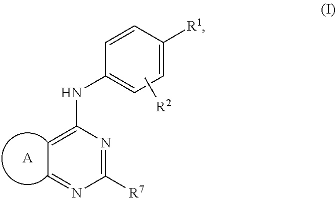
or a pharmaceutically acceptable salt thereof, wherein:
R¹ is selected from C₂₋₆alkyl; C₂₋₆alkenyl; C₂₋₆alkynyl; C₂₋₆alkoxy; C₂₋₆alkenyloxy; C₂₋₆alkynyloxy; C₃₋₇cycloalkyl; —O—C₃₋₇cycloalkyl; C₆₋₁₀aryl; —O—(CH₂)ₘ—C₆₋₁₀aryl; 6 membered heteroaryl; and thiophenyl; wherein alkyl, alkenyl, alkynyl, alkoxy, alkenyloxy, alkynyloxy and cycloalkyl are optionally substituted with 1, 2 or 3 substituents selected from —F and —CF₃ and wherein aryl and heteroaryl are optionally substituted with 1 or 2 substituents selected from -halo, —C₁₋₃alkyl, —C₁₋₈alkoxy and —C₂₋₈alkynyloxy wherein —C₁₋₃alkyl, —C₁₋₈alkoxy and —C₂₋₈alkynyloxy are optionally substituted with 1, 2, or 3 substituents selected from —F, —CF₃, —NHC(O)O—C₁₋₆alkyl or two substituents together with the carbon to which they are attached form diazirinyl;
R² is selected from —H; -halo; and —C₁₋₃alkyl optionally substituted with 1, 2 or 3 substituents selected from —F and —CF₃;
A is selected from 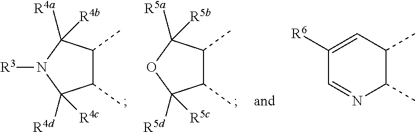
or a N-oxide thereof;
R³ is selected from —H; —C₁₋₆alkyl; —C₂₋₆alkenyl; —C₂₋₆alkynyl; C₃₋₇cycloalkyl; and a 5 or 6 membered heterocycloalkyl; wherein the alkyl, alkenyl, alkynyl, cycloalkyl or heterocycloalkyl are optionally substituted by 1, 2 or 3 groups selected from —F, —CF₃, —C₁₋₃alkyl optionally substituted by 1 or 2 substituents selected from —F, —CF₃, —C(O)NR⁸R⁹ and —NR⁸R⁹;
R^{4a} and R^{4b} are each independently selected from —H and —C₁₋₃ alkyl optionally substituted with 1, 2 or 3 substituents selected from —F and CF₃;
R^{4c} and R^{4d} are each independently selected from —H and —C₁₋₃ alkyl optionally substituted with 1, 2 or 3 substituents selected from —F and CF₃, or R^{4c} and R^{4d} together with the carbon to which they are attached represent carbonyl;
R^{5a}, R^{5b}, R^{5c} and R^{5d} are each independently selected from —H and —C₁₋₃ alkyl optionally substituted with 1, 2 or 3 substituents selected from —F and CF₃;
R⁶ is selected from —H; -halo; —NH₂; —CN; —C₁₋₃alkyl optionally substituted with 1, 2 or 3 substituents selected from —F and CF₃; —C₁₋₃alkoxy optionally substituted with 1, 2 or 3 substituents selected from —F and —CF₃; —C(O)O—C₁₋₃alkyl; —C(O)NR⁸R⁹; —C(O)OH; and —NHC(O)—C₁₋₃ alkyl;
R⁷ is selected from NR¹⁰R¹¹; a 5 to 7 membered monocyclic heterocycloalkyl; and a 5 or 6 membered monocyclic heteroaryl; wherein the heterocycloalkyl and heteroaryl are optionally substituted with 1, 2 or 3 groups selected from —CN; —C₁₋₆alkyl optionally substituted with 1, 2 or 3 substituents selected from —F, —CF₃ and —OH; —C₁₋₃alkoxy optionally substituted with 1, 2 or 3 substituents selected from —F and CF₃; —C(O)OH; —C₁₋₃alkylene-NHC(O)C₁₋₆alkyl; —C₁₋₃alkylene-NHC(O)OC₁₋₆alkyl; C₃₋₅cycloalkyl; or the heterocycloalkyl is optionally substituted with two substituents on the same ring carbon which together with the carbon atom to which they are attached form a 5 to 7 membered monocyclic heterocycloalkyl; and wherein when R⁷ is morpholinyl and R¹ is unsubstituted phenyl, R² is not —H;
R⁸ and R⁹ are each independently selected from —H and —C₁₋₆ alkyl;
R¹⁰ is —C₁₋₆ alkyl;
R¹¹ is selected from —C₁₋₆alkyl optionally substituted with 1 or 2 substituents selected from —F and —C₁₋₃alkoxy; and —(CH₂)ₜR¹²;
R¹² is a 5 or 6 membered heteroaryl, a 3 to 5 membered cycloalkyl or a 3 to 6 membered heterocycloalkyl;
m is 0 or 1; and
n is 1, 2 or 3;
in combination with pridopidine, or a pharmaceutically acceptable salt thereof.

22. The pharmaceutical composition according to claim 21, wherein the amount of a compound according to Formula (I), or a pharmaceutically acceptable salt thereof, in the pharmaceutical composition is from about 1 mg to about 1500 mg.

23. The pharmaceutical composition according to claim 21, wherein the compound according to Formula (I), or a pharmaceutically acceptable salt thereof, is Compound A:

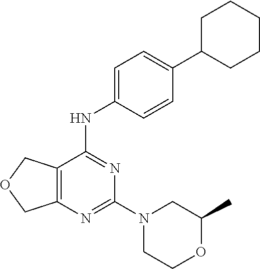 [SMILES: C[C@@H]1CN(c2nc3c(c(Nc4ccc(C5CCCCC5)cc4)n2)COC3)CCO1]

24. The pharmaceutical composition according to claim 21, wherein the compound according to Formula (I), or a pharmaceutically acceptable salt thereof, is Compound B:

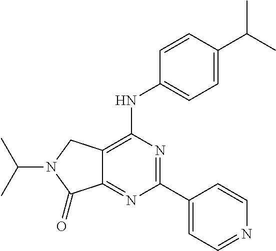 [SMILES: CC(C)c1ccc(Nc2nc(-c3ccncc3)nc3c2CN(C(C)C)C3=O)cc1]

25. The pharmaceutical composition according to claim 21, wherein the compound according to Formula (I), or a pharmaceutically acceptable salt thereof, is Compound C:

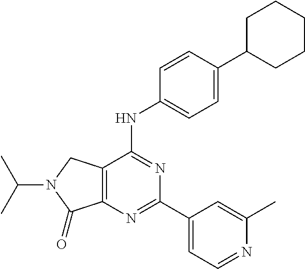 [SMILES: Cc1cc(-c2nc(Nc3ccc(C4CCCCC4)cc3)c3c(n2)C(=O)N(C(C)C)C3)ccn1]

26. The pharmaceutical composition according to claim 21 wherein the compound according to Formula (I), or a pharmaceutically acceptable salt thereof, is Compound D:

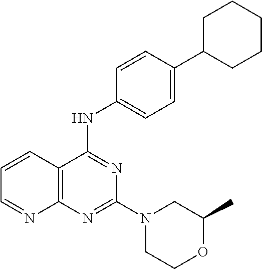 [SMILES: C[C@@H]1CN(c2nc(Nc3ccc(C4CCCCC4)cc3)c3cccnc3n2)CCO1]

27. The pharmaceutical composition according to claim 21, wherein the compound according to Formula (I), or a pharmaceutically acceptable salt thereof, is Compound E:

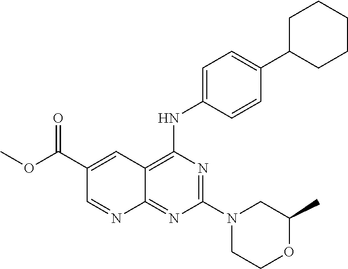 [SMILES: COC(=O)c1cnc2nc(N3CCO[C@H](C)C3)nc(Nc3ccc(C4CCCCC4)cc3)c2c1]

28. The pharmaceutical composition according to claim 21, wherein the compound according to Formula (I), or a pharmaceutically acceptable salt thereof, is Compound F:

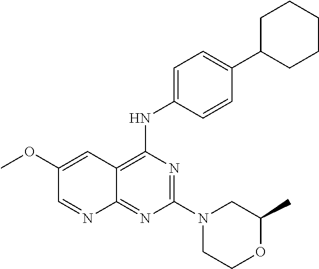 [SMILES: COc1cnc2nc(N3CCO[C@H](C)C3)nc(Nc3ccc(C4CCCCC4)cc3)c2c1]

29. The pharmaceutical composition according to claim 21, wherein the compound according to Formula (I), or a pharmaceutically acceptable salt thereof, is Compound G:

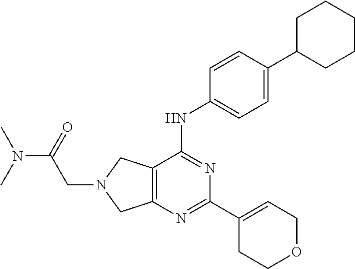 [SMILES: CN(C)C(=O)CN1Cc2nc(C3=CCOCC3)nc(Nc3ccc(C4CCCCC4)cc3)c2C1]

30. The pharmaceutical composition according to claim 21, wherein the compound according to Formula (I), or a pharmaceutically acceptable salt thereof, is Compound H:

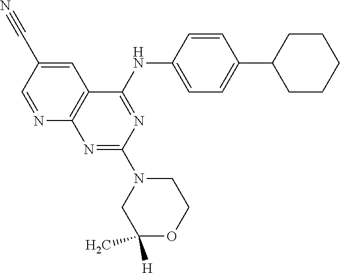 [SMILES: C[C@@H]1CN(c2nc(Nc3ccc(C4CCCCC4)cc3)c3cc(C#N)cnc3n2)CCO1]

31. The pharmaceutical composition according to claim 21, wherein the amount of pridopidine, or a pharmaceutically acceptable salt thereof, in the pharmaceutical composition is from about 0.1 mg to about 1000 mg.
